SEQUENCE LISTING (88 sequences)

SEQ ID NO: 1 - protein, 8 aa, synthetic construct
GLTFGSYA

SEQ ID NO: 2 - protein, 7 aa, synthetic construct
ISSRGVT

SEQ ID NO: 3 - protein, 13 aa, synthetic construct
NAKGPFMLREYDY

SEQ ID NO: 4 - protein, 8 aa, synthetic construct
GRSISDYF

SEQ ID NO: 5 - protein, 8 aa, synthetic construct
ISWSGGTA

SEQ ID NO: 6 - protein, 20 aa, synthetic construct
AARLSYDSRTSLQNSYDYNY

SEQ ID NO: 7 - protein, 8 aa, synthetic construct
GRTVSTYN

SEQ ID NO: 8 - protein, 8 aa, synthetic construct
INWTGGST

SEQ ID NO: 9 - protein, 12 aa, synthetic construct
AATRASWTENTY

SEQ ID NO: 10 - protein, 8 aa, synthetic construct
GRAFKWWT

SEQ ID NO: 11 - protein, 8 aa, synthetic construct
ISWSDGST

SEQ ID NO: 12 - protein, 20 aa, synthetic construct
ARYGKDLSGRHSKNVQDYDY

SEQ ID NO: 13 - protein, 8 aa, synthetic construct
ASTFRFFA

SEQ ID NO: 14 - protein, 7 aa, synthetic construct
ISRDGDA

SEQ ID NO: 15 - protein, 9 aa, synthetic construct
HTRILSDHY

SEQ ID NO: 16 - protein, 8 aa, synthetic construct
GRTVSAYN

SEQ ID NO: 17 - protein, 8 aa, synthetic construct
INWTGGGT

SEQ ID NO: 18 - protein, 12 aa, synthetic construct
AATRTSWTENTY

SEQ ID NO: 19 - protein, 8 aa, synthetic construct
GRTVSTYN

SEQ ID NO: 20 - protein, 8 aa, synthetic construct
INWTGGGT

SEQ ID NO: 21 - protein, 12 aa, synthetic construct
AATRTSWTENTY

SEQ ID NO: 22 - protein, 8 aa, synthetic construct
GRDFSSHF

SEQ ID NO: 23 - protein, 8 aa, synthetic construct
ISWSGGTT

SEQ ID NO: 24 - protein, 20 aa, synthetic construct
AARRTYDSRTSLRNSYDYPY

SEQ ID NO: 25 - protein, 8 aa, synthetic construct
GRTVSTYN

SEQ ID NO: 26 - protein, 8 aa, synthetic construct
INWTGGST

SEQ ID NO: 27 - protein, 12 aa, synthetic construct
AATRTSWTENTY

SEQ ID NO: 28 - protein, 7 aa, synthetic construct
GIVRASG

SEQ ID NO: 29 - protein, 8 aa, synthetic construct
ITTGGETT

SEQ ID NO: 30 - protein, 5 aa, synthetic construct
NQRSY

SEQ ID NO: 31 - protein, 8 aa, synthetic construct
GSIWSASG

SEQ ID NO: 32 - protein, 8 aa, synthetic construct
ITTGGETT

SEQ ID NO: 33 - protein, 5 aa, synthetic construct
NSRSY

SEQ ID NO: 34 - protein, 8 aa, synthetic construct
GGIGSLSN

SEQ ID NO: 35 - protein, 7 aa, synthetic construct
ITGQSTT

SEQ ID NO: 36 - protein, 9 aa, synthetic construct
NVATLRNEY

SEQ ID NO: 37 - protein, 8 aa, synthetic construct
GRTFSASA

SEQ ID NO: 38 - protein, 8 aa, synthetic construct
VAWGGRDT

SEQ ID NO: 39 - protein, 17 aa, synthetic construct
AAGQRGWSHSTSRDYDY

SEQ ID NO: 40 - protein, 8 aa, synthetic construct
GRTFSASA

SEQ ID NO: 41 - protein, 8 aa, synthetic construct
VAWGGRDT

SEQ ID NO: 42 - protein, 17 aa, synthetic construct
AAGQRGWSHSTSSDYDY

SEQ ID NO: 43 - protein, 8 aa, synthetic construct
GSIRSASG

SEQ ID NO: 44 - protein, 8 aa, synthetic construct
ITTSGETT

SEQ ID NO: 45 - protein, 5 aa, synthetic construct
NLRSY

SEQ ID NO: 46 - protein, 119 aa, synthetic construct
  VARIANT 3: note = Q or K
  VARIANT 5: note = V or E
QVXLXGSGGGLVRAGGSLRLSCAGSGLTFGSYAMAWYRQAPGKQRELVASISSRGVTNYADSVEGRFTISRDNAKSTVYLQMNDLKPEDAAVYYCNAKGPFMLREYDYWGQGTQVTVSS

SEQ ID NO: 47 - protein, 127 aa, synthetic construct
QVKLEESGGGLVQAGGSLRISCAASGRSISDYFMGWFRQTPGKEREFVAAISWSGGTAYYADSVKGRFTISRDNAKNTGYLQMNSLKPEDTAVYFCAARLSYDSRTSLQNSYDYNYWGQGTQVTVSS

SEQ ID NO: 48 - protein, 119 aa, synthetic construct
DVQLVDSGGGLVQAGGSLRLSCAASGRTVSTYNMAWFRQAPGKEREIVAVINWTGGSTYYAESVKGRFTISGDNPKNTVYLQMNSLKPEDTAVYYCAATRASWTENTYWGQGTQVTVSS

SEQ ID NO: 49 - protein, 127 aa, synthetic construct
EVQLVESGGRLVQAGGSLRLSCVASGRAFKWWTMGWFRQAPGKEREFVASISWSDGSTTYGDSVKGRFAISRDNNKNTVYLQMNSLTPEDTAVYYCARYGKDLSGRHSKNVQDYDYWGQGTQVTVSS

SEQ ID NO: 50 - protein, 115 aa, synthetic construct
  VARIANT 3: note = Q or K
  VARIANT 5: note = V or E
QVXLXDSGGGLVQPGGSLRLSCTTSASTFRFFAMHWYRQAPGKERELVAVISRDGDAEYVDSVKGRFTISRDNAKNAVYLQMNSLKPEDTAVYYCHTRILSDHYWGQGTQVTVSS

SEQ ID NO: 51 - protein, 119 aa, synthetic construct
QVKLEESGGGLVQPGGSLRLSCAASGRTVSAYNMAWFRQAPGKEREIVAVINWTGGGTYYAESVKGRFTISRDNPKNTVYLQMNSLKPEDTAVYYCAATRTSWTENTYWGQGTQVTVSS

SEQ ID NO: 52 - protein, 119 aa, synthetic construct
  VARIANT 3: note = Q or K
  VARIANT 5: note = V or E
QVXLXESGGGLVQPGGSLRLSCAASGRTVSTYNMAWFRQAPGKEREIVAVINWTGGGTYYAESVKGRFTISRDNPKNTVYLQMNSLKPEDTAVYYCAATRTSWTENTYWGQGTQVTVSS

SEQ ID NO: 53 - protein, 127 aa, synthetic construct
  VARIANT 3: note = Q or K
  VARIANT 5: note = V or E
QVXLXESGGGLVQAGGSLRISCVASGRDFSSHFMGWSRQAPGKEREFVAAISWSGGTTYYADSVKGRFAISRDNDKTTVYLQMNSLKPEDSAVYYCAARRTYDSRTSLRNSYDYPYWGQGTQVTVSS

SEQ ID NO: 54 - protein, 119 aa, synthetic construct
  VARIANT 3: note = Q or K
  VARIANT 5: note = V or E
QVXLXESGGGLVQAGGSLRLSCAASGRTVSTYNMAWFRQAPGKEREIVAVINWTGGSTYYAESVKGRFTISRDNPKNTVYLQMNSLKPEDTAVYYCAATRTSWTENTYWGQGTQVTVSS

SEQ ID NO: 55 - protein, 111 aa, synthetic construct
  VARIANT 3: note = Q or K
  VARIANT 5: note = V or E
QVXLXESGGGLVQPGGSLRLSCTASGIVRASGMAWYRQAPGKQRELVASITTGGETTWYADSVKGRFTISIDNAKMMLYLQMNALKPEDTAVYLCNQRSYWGQGTQVTVSS

SEQ ID NO: 56 - protein, 114 aa, synthetic construct
QVKLEESGGGLVQPGGSLRLSCTASGSIWSASGLTWYRQRPGNERELVASITTGGETTWYADSVKGRFTISISRDNGKMITYLQMNSLKPEDTAVYLCNSRSYWGQGTQVTVSS

SEQ ID NO: 57 - protein, 115 aa, synthetic construct
  VARIANT 3: note = Q or K
  VARIANT 5: note = V or E
QVXLXESGGGLVQPGGSLRLACAASGGIGSLSNVRWYRQGPGKEREYVAGITGQSTTYYADSVKGRFTISRDNVKNTVDLQMNSLKPEDTAVYICNVATLRNEYWGQGTQVTVSS

SEQ ID NO: 58 - protein, 124 aa, synthetic construct
  VARIANT 3: note = Q or K
  VARIANT 5: note = V or E
QVXLXESGGGLVQAGGSLSLSCAASGRTFSASAMAWFRQAPGTDREFVAAVAWGGRDTHYADSVKGRFTISMNNAKNTVVLQMNSLKPEDTAVYYCAAGQRGWSHSTSRDYDYWGQGTQVTVSS

SEQ ID NO: 59 - protein, 124 aa, synthetic construct
  VARIANT 3: note = Q or K
  VARIANT 5: note = V or E
QVXLXESGGGLVQAGGSLSLSCAASGRTFSASAMAWFRQAPGTDREFVAAVAWGGRDTHYADSVKGRFTISMNNAKNTVVLQMNSLKPEDTAVYYCAAGQRGWSHSTSSDYDYWGQGTQVTVSS

SEQ ID NO: 60 - protein, 112 aa, synthetic construct
  VARIANT 3: note = Q or K
  VARIANT 5: note = V or E
QVXLXESGGGLVQAGGSLRLSCAASGSIRSASGMHWYRQAPGKERELVASITTSGETTWYRDSVKGRFTISINNAKMMLYLQMNSLKFEDTAVYYCNLRSYWGQGTQVTVSS

SEQ ID NO: 61 - protein, 364 aa, synthetic construct
MLRLLLALNLFPSIQVTGQVQLVESGGGLVQPGGSLRLSCAASGRTDSTYNMAWFRQAPGKEREIVAVINWTGGGTYYAESVKGRFTISRDNPKNTVYLQMNSLKPEDTAVYYCAATRTSWTENTYWGQGTQVTVSSPLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 62 - protein, 395 aa, synthetic construct
MLRLLLALNLFPSIQVTGQVQLVESGGGLVQPGGSLRLSCAASGRTDSTYNMAWFRQAPGKEREIVAVINWTGGGTYYAESVKGRFTISRDNPKNTVYLQMNSLKPEDTAVYYCAATRTSWTENTYWGQGTQVTVSSPLRDDGGGGSGGGGSGGGGSGGTTTPAPRPPTPAPTIASQPLSLRPEACRPAAGGAVHTRGLDFACDVVSPSKPFWVLVVVGGVLACYSLLVTVAFIIFWVRKRGRKKLLYIFKQPFMRPVQTTQEEDGCSCRFPEEEEGGCELLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 63 - protein, 403 aa, synthetic construct
MLRLLLALNLFPSIQVTGQVQLVESGGGLVQAGGSLRISCVASGRDFSSHFMGWSRQAPGKEREFVAAISWSGGTTYYADSVKGRFAISRDNDKTTVYLQMNSLKPEDSAVYYCAARRTYDSRTSLRNSYDYPYWGQGTQVTVSSPLRDDGGGGSGGGGSGGGGSGGTTTPAPRPPTPAPTIASQPLSLRPEACRPAAGGAVHTRGLDFACDVVSPSKPFWVLVVVGGVLACYSLLVTVAFIIFWVRKRGRKKLLYIFKQPFMRPVQTTQEEDGCSCRFPEEEEGGCELLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 64 - protein, 372 aa, synthetic construct
MLRLLLALNLFPSIQVTGQVQLVESGGGLVQAGGSLRISCVASGRDFSSHFMGWSRQAPGKEREFVAAISWSGGTTYYADSVKGRFAISRDNDKTTVYLQMNSLKPEDSAVYYCAARRTYDSRTSLRNSYDYPYWGQGTQVTVSSPLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 65 - protein, 395 aa, synthetic construct
MLRLLLALNLFPSIQVTGQVQLVESGGGLVQAGGSLRLSCAASGRTVSTYNMAWFRQAPGKEREIVAVINWTGGSTYYAESVKGRFTISRDNPKNTVYLQMNSLKPEDTAVYYCAATRTSWTENTYWGQGTQVTVSSPLRDDGGGGSGGGGSGGGGSGGTTTPAPRPPTPAPTIASQPLSLRPEACRPAAGGAVHTRGLDFACDVVSPSKPFWVLVVVGGVLACYSLLVTVAFIIFWVRKRGRKKLLYIFKQPFMRPVQTTQEEDGCSCRFPEEEEGGCELLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 66 - protein, 364 aa, synthetic construct
MLRLLLALNLFPSIQVTGQVQLVESGGGLVQAGGSLRLSCAASGRTVSTYNMAWFRQAPGKEREIVAVINWTGGSTYYAESVKGRFTISRDNPKNTVYLQMNSLKPEDTAVYYCAATRTSWTENTYWGQGTQVTVSSPLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 67 - protein, 839 aa, synthetic construct
ATGCTCAGGCTGCTCTTGGCTCTCAACTTATTCCCTTCAATTCAAGTAACAGGAABDSEQUENCECCTTTGCGAGACGACGGTGGCGGGGGATCAGGTGGTGGAGGTAGCGGGGGAGGGGGCTCAGGCGGTACAACTACGCCTGCACCTCGCCCACCGACCCCAGCACCAACCATCGCTTCACAGCCTTTGAGCCTGCGACCAGAGGCATGTCGCCCTGCTGCGGGCGGTGCCGTTCATACTCGCGGACTTGATTTTGCGTGTGACGTCGTCTCGCCTTCTAAGCCCTTTTGGGTGCTGGTGGTGGTTGGTGGAGTCCTGGCTTGCTATAGCTTGCTAGTAACAGTGGCCTTTATTATTTTCTGGGTGAGGAAACGGGGCAGAAAGAAACTCCTGTATATATTCAAACAACCATTTATGCGACCAGTACAAACTACTCAAGAGGAAGATGGCTGTAGCTGCCGATTTCCAGAAGAAGAAGAAGGAGGATGTGAACTGCTGAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGCGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGACTCAGTACAGCCACCAAGGACACCTACGACGCCCTTCACATGCAGGCCCTGCCCCCTCGC

SEQ ID NO: 68 - protein, 18 aa, synthetic construct
MLRLLLALNLFPSIQVTG

SEQ ID NO: 69 - protein, 17 aa, synthetic construct
GGGGSGGGGSGGGGSGG

SEQ ID NO: 70 - protein, 45 aa, synthetic construct
TTTPAPRPPTPAPTIASQPLSLRPEACRPAAGGAVHTRGLDFACD

SEQ ID NO: 71 - protein, 24 aa, synthetic construct
IYIWAPLAGTCGVLLLSLVITLYC

SEQ ID NO: 72 - protein, 32 aa, synthetic construct
PSKPFWVLVVVGGVLACYSLLVTVAFIIFWVR

SEQ ID NO: 73 - protein, 42 aa, synthetic construct
KRGRKKLLYIFKQPFMRPVQTTQEEDGCSCRFPEEEEGGCEL

SEQ ID NO: 74 - protein, 114 aa, synthetic construct
LRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 75 - protein, 5 aa, synthetic construct
GGGGS

SEQ ID NO: 76 - protein, 502 aa, synthetic construct
MLRLLLALNLFPSIQVTGQVQLVESGGGLVQPGGSLRLSCSVSLSDFGSKHLAWHRQAPGKEREWVALIFSSGATNYANSVKGRFTISRDNSKNTVYLQMNSLKPEDTAVYYCNLVPPSPAWGQGTQVTVSSPSGGGGQVQVQLVESGGGLVQAGGSLRISCVASGRDFSSHFMGWSRQAPGKEREFVAAISWSGGTTYYADSVKGRFAISRDNDKTTVYLQMNSLKPEDSAVYYCAARRTYDSRTSLRNSYDYPYWGQGTQVTVSSPSTTTPAPRPPTPAPTIASQPLSLRPEACRPAAGGAVHTRGLDFACDPSKPFWVLVVVGGVLACYSLLVTVAFIIFWVRKRGRKKLLYIFKQPFMRPVQTTQEEDGCSCRFPEEEEGGCELLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 77 - protein, 1215 aa, synthetic construct
ATGGAGTTTGGGCTGAGCTGGGTTTTCCTCGTTGCTCTTTTTAGAGGTGTCCAGTGTACAGGAGGGTCTTCGCAGGTACAGCTGGTGGAGTCTGGGGGAGGATTGGTGCAGGCTGGGGGCTCTCTGAGACTCTCCTGTGCAGCCTCTGGACGCACCGTCAGTACCTATAATATGGCCTGGTTCCGCCAGGCTCCAGGGAAGGAGCGTGAGATTGTAGCAGTTATTAACTGGACTGGTGGTAGCACATACTATGCAGAGTCCGTGAAGGGCCGATTCACCATCTCCAGAGATAACCCCAAGAACACGGTGTATTTACAAATGAACAGCCTGAAACCTGAGGACACGGCCGTTTATTACTGTGCAGCAACTAGGACATCCTGGACCGAGAATACCTACTGGGGCCAGGGGACCCAGGTCACCGTTTCCTCAGAAGACTTCCTTGGAGGAGGCGGAAGTCAAGTGCAGTTGCAACAACCAGGTGCCGAGCTTGGGAAACCGGGTACCAGCGTTAAACTCTCTTGCAAAGCATCCGGTTATACCTTTACTTCTTACTGGATCCATTGGGTCAAACAACGCCCAGGCCAGGGTCTCGAATGGGTGGGCAACATCAACCCAAACAGCGGCTCTATCAATTATAACGAAAAGTTTAAGAATAAAGCAACTCTCACGGTGGATAAGAGCAGCTCAACCGCTTATATGCAATTGAGTACTTTGACCAGTGAAGATTTCGCTGTGTATTATTGCACGCGCGATACATCCGGACAGTATTATTTCGATTATTGGGGCCAGGGGACAACCCTGACAGTGTCTAGCGGCGGTGGTGGGTCAGGCGGCGGTGGGAGCGGAGGAGGTGGAAGCGATATCGTAATGAGTCAATCCCCTAGCAGCTTGGCGGTAAGTGCTGGGGAACGCGTTACAATGTCCTGTAAGAGCTCTCAATCTTTGCTGAATTCCAGGACTAGGAAGAATTATCTGGCCTGGTATCAACAAAAGCCCGGACAAAGTCCAAAGCTCCTGATTTATTGGGCTAGCACCAGAGAGTCAGGAGTACCAGACCGGTTTACTGGAAGCGGGTCAGGAACCGACTTTACCCTGACAATATCCAGCGTCCAAGCAGAAGATCTTGCCGTGTACTATTGTATTCAGTCCTACCACTTGCGCACTTTTGGGGGGGGAACAAAACTCGAGATAAAGCACCACCACCACCACCACTAG

SEQ ID NO: 78 - protein, 1200 aa, synthetic construct
ATGGAGTTTGGGCTGAGCTGGGTTTTCCTCGTTGCTCTTTTTAGAGGTGTCCAGTGTACAGGACAAGTGCAGTTGCAACAACCAGGTGCCGAGCTTGGGAAACCGGGTACCAGCGTTAAACTCTCTTGCAAAGCATCCGGTTATACCTTTACTTCTTACTGGATCCATTGGGTCAAACAACGCCCAGGCCAGGGTCTCGAATGGGTGGGCAACATCAACCCAAACAGCGGCTCTATCAATTATAACGAAAAGTTTAAGAATAAAGCAACTCTCACGGTGGATAAGAGCAGCTCAACCGCTTATATGCAATTGAGTACTTTGACCAGTGAAGATTTCGCTGTGTATTATTGCACGCGCGATACATCCGGACAGTATTATTTCGATTATTGGGGCCAGGGGACAACCCTGACAGTGTCTAGCGGCGGTGGTGGGTCAGGCGGCGGTGGGAGCGGAGGAGGTGGAAGCGATATCGTAATGAGTCAATCCCCTAGCAGCTTGGCGGTAAGTGCTGGGGAACGCGTTACAATGTCCTGTAAGAGCTCTCAATCTTTGCTGAATTCCAGGACTAGGAAGAATTATCTGGCCTGGTATCAACAAAAGCCCGGACAAAGTCCAAAGCTCCTGATTTATTGGGCTAGCACCAGAGAGTCAGGAGTACCAGACCGGTTTACTGGAAGCGGGTCAGGAACCGACTTTACCCTGACAATATCCAGCGTCCAAGCAGAAGATCTTGCCGTGTACTATTGTATTCAGTCCTACCACTTGCGCACTTTTGGGGGGGGAACAAAACTCGAGATAAAGGGCCTTGGAGGAGGCGGAAGTCAGGTACAGCTGGTGGAGTCTGGGGGAGGATTGGTGCAGGCTGGGGGCTCTCTGAGACTCTCCTGTGCAGCCTCTGGACGCACCGTCAGTACCTATAATATGGCCTGGTTCCGCCAGGCTCCAGGGAAGGAGCGTGAGATTGTAGCAGTTATTAACTGGACTGGTGGTAGCACATACTATGCAGAGTCCGTGAAGGGCCGATTCACCATCTCCAGAGATAACCCCAAGAACACGGTGTATTTACAAATGAACAGCCTGAAACCTGAGGACACGGCCGTTTATTACTGTGCAGCAACTAGGACATCCTGGACCGAGAATACCTACTGGGGCCAGGGGACCCAGGTCACCGTTTCCTCACACCACCACCACCACCACTAG

SEQ ID NO: 79 - protein, 404 aa, synthetic construct
MEFGLSWVFLVALFRGVQCTGGSSQVQLVESGGGLVQAGGSLRLSCAASGRTVSTYNMAWFRQAPGKEREIVAVINWTGGSTYYAESVKGRFTISRDNPKNTVYLQMNSLKPEDTAVYYCAATRTSWTENTYWGQGTQVTVSSEDFLGGGGSQVQLQQPGAELGKPGTSVKLSCKASGYTFTSYWIHWVKQRPGQGLEWVGNINPNSGSINYNEKFKNKATLTVDKSSSTAYMQLSTLTSEDFAVYYCTRDTSGQYYFDYWGQGTTLTVSSGGGGSGGGGSGGGGSDIVMSQSPSSLAVSAGERVTMSCKSSQSLLNSRTRKNYLAWYQQKPGQSPKLLIYWASTRESGVPDRFTGSGSGTDFTLTISSVQAEDLAVYYCIQSYHLRTFGGGTKLEIKHHHHHH

SEQ ID NO: 80 - protein, 399 aa, synthetic construct
MEFGLSWVFLVALFRGVQCTGQVQLQQPGAELGKPGTSVKLSCKASGYTFTSYWIHWVKQRPGQGLEWVGNINPNSGSINYNEKFKNKATLTVDKSSSTAYMQLSTLTSEDFAVYYCTRDTSGQYYFDYWGQGTTLTVSSGGGGSGGGGSGGGGSDIVMSQSPSSLAVSAGERVTMSCKSSQSLLNSRTRKNYLAWYQQKPGQSPKLLIYWASTRESGVPDRFTGSGSGTDFTLTISSVQAEDLAVYYCIQSYHLRTFGGGTKLEIKGLGGGGSQVQLVESGGGLVQAGGSLRLSCAASGRTVSTYNMAWFRQAPGKEREIVAVINWTGGSTYYAESVKGRFTISRDNPKNTVYLQMNSLKPEDTAVYYCAATRTSWTENTYWGQGTQVTVSSHHHHHH

SEQ ID NO: 81 - protein, 246 aa, synthetic construct
QVQLQQPGAELGKPGTSVKLSCKASGYTFTSYWIHWVKQRPGQGLEWVGNINPNSGSINYNEKFKNKATLTVDKSSSTAYMQLSTLTSEDFAVYYCTRDTSGQYYFDYWGQGTTLTVSSGGGGSGGGGSGGGGSDIVMSQSPSSLAVSAGERVTMSCKSSQSLLNSRTRKNYLAWYQQKPGQSPKLLIYWASTRESGVPDRFTGSGSGTDFTLTISSVQAEDLAVYYCIQSYHLRTFGGGTKLEIK

SEQ ID NO: 82 - DNA, 858 bp, synthetic construct
  source 1..858: mol_type = other DNA
  misc_feature 72^73: note = coding sequence for an sdAb
atggagtttgggctgagctgggttttcctcgttgctctttttagaggtgtccagtgtacaggagggtcttcggaagacttccttggaggaggcggaagtcaagtgcagttgcaacaaccaggtgccgagcttgggaaaccgggtaccagcgttaaactctcttgcaaagcatccggttatacctttacttcttactggatccattgggtcaaacaacgcccaggccagggtctcgaatgggtgggcaacatcaacccaaacagcggctctatcaattataacgaaaagtttaagaataaagcaactctcacggtggataagagcagctcaaccgcttatatgcaattgagtactttgaccagtgaagatttcgctgtgtattattgcacgcgcgatacatccggacagtattatttcgattattggggccaggggacaaccctgacagtgtctagcggcggtggtgggtcaggcggcggtgggagcggaggaggtggaagcgatatcgtaatgagtcaatcccctagcagcttggcggtaagtgctggggaacgcgttacaatgtcctgtaagagctctcaatctttgctgaattccaggactaggaagaattatctggcctggtatcaacaaaagcccggacaaagtccaaagctcctgatttattgggctagcaccagagagtcaggagtaccagaccggtttactggaagcgggtcaggaaccgactttaccctgacaatatccagcgtccaagcagaagatcttgccgtgtactattgtattcagtcctaccacttgcgcacttttggggggggaacaaaactcgagataaagcaccaccaccaccaccactag

SEQ ID NO: 83 - protein, 699 aa, synthetic construct
MDWTWRFLFVVAAATGVQSTGQVQLVESGGGLVQAGGSLRLSCAASGRTVSTYNMAWFRQAPGKEREIVAVINWTGGSTYYAESVKGRFTISRDNPKNTVYLQMNSLKPEDTAVYYCAATRTSWTENTYWGQGTQVTVSSPLGRPGGGGSGGGGSGGGGSGGTTTPAPRPPTPAPTIASQPLSLRPEACRPAAGGAVHTRGLDFACDGSLPSGGGGSDIVMSQSPSSLAVSAGERVTMSCKSSQSLLNSRTRKNYLAWYQQKPGQSPKLLIYWASTRESGVPDRFTGSGSGTDFTLTISSVQAEDLAVYYCIQSYHLRTFGGGTKLEIKGGGGSGGGGSGGGGSQVQLQQPGAELGKPGTSVKLSCKASGYTFTSYWIHWVKQRPGQGLEWVGNINPNSGSINYNEKFKNKATLTVDKSSSTAYMQLSTLTSEDFAVYYCTRDTSGQYYFDYWGQGTTLTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 84 - protein, 231 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 85 - protein, 136 aa, synthetic construct
  NON_TER 136: note = NK-engaging antibody fragment attached at C-terminus
QVQLVESGGGLVQAGGSLRLSCAASGRTVSTYNMAWFRQAPGKEREIVAVINWTGGSTYYAESVKGRFTISRDNPKNTVYLQMNSLKPEDTAVYYCAATRTSWTENTYWGQGTQVTVSSGGGGSGGGGSGGGGSAS

SEQ ID NO: 86 - protein, 8 aa, synthetic construct
  VARIANT 3: note = X is S or D
  VARIANT 4: note = X is I or F
  VARIANT 6: note = X is D or S
  VARIANT 7: note = X is Y or H
GRXXSXXF

SEQ ID NO: 87 - protein, 8 aa, synthetic construct
  VARIANT 8: note = X is A or T
ISWSGGTX

SEQ ID NO: 88 - protein, 20 aa, synthetic construct
  VARIANT 4: note = X is L or R
  VARIANT 5: note = X is S or T
  VARIANT 13: note = X is Q or R
  VARIANT 19: note = X is N or P
AARXXYDSRTSLXNSYDYXY

(19) United States
(12) Patent Application Publication
(10) Pub. No.: US 2026/0274984 A1
(43) Pub. Date: Sep. 17, 2026

(54) ANTI-MESOTHELIN (MSLN) SINGLE DOMAIN ANTIBODIES AND THERAPEUTIC CONSTRUCTS

(71) Applicant: NATIONAL RESEARCH COUNCIL OF CANADA, Ottawa (CA)

(72) Inventors: Mehdi ARBABI-GHAHROUDI, Ottawa (CA); Scott MCCOMB, Ottawa (CA); Robert PON, Gatineau (CA); Risini WEERATNA, Ottawa (CA)

(21) Appl. No.: 19/166,949

(22) PCT Filed: Mar. 25, 2024
(86) PCT No.: PCT/CA2024/050361

§ 371 Date: Sep. 19, 2025

Related U.S. Application Data
(60) Provisional application No. 63/491,827, filed on Mar. 23, 2023.

(51) Int. Cl. C07K 16/30; A61K 39/00; A61K 40/11; A61K 40/31; A61K 40/42; A61P 35/00; C12N 5/0783

(52) U.S. Cl. CPC C07K 16/30; A61K 40/11; A61K 40/31; A61K 40/4255; A61P 35/00; C12N 5/0636; A61K 2039/505; C07K 2317/31; C07K 2317/53; C07K 2317/565; C07K 2317/569; C07K 2317/622; C07K 2317/92; C07K 2319/02; C07K 2319/03; C12N 2510/00

(57) ABSTRACT

The present disclosure provides anti-MSLN single domain antibodies (sdAb), as well as constructs comprising the sdAbs. The constructs include multivalent antibodies comprising any one of the sdAbs, such constructs including bispecific T-cell engagers (BiTEs), bispecific natural killer cell engagers (BiKEs), and trispecific killer cell engagers (TrikEs). Also described are chimeric antigen receptors (CARs) for CAR-T and CAR-NK therapy comprising any one of the aforementioned sdAbs. Uses of these molecules in the treatment of cancers (in particular, solid cancers) are also described.

### CROSS REFERENCE TO RELATED APPLICATIONS

This application claims the benefit of U.S. Provisional Patent Application No. 63/491,827, filed Mar. 23, 2023, which is incorporated by reference herein in its entirety.

### FIELD

The present disclosure relates generally to anti-MSLN antibodies. More particularly, the present disclosure relates to anti-MSLN single domain antibodies.

### BACKGROUND

Cancer is a major public health problem and the second leading cause of death worldwide. Traditional therapy for cancer has included surgery, radiation and chemotherapy. These have been moderately successful for treatment of some cancers, particularly those diagnosed at early stages. However effective therapy is lacking for many aggressive cancers as tumor specific biomarkers is scarce.

Immunotherapy; harnessing a patient's own immune system to recognize and kill cancer, is now considered the fourth pillar of cancer therapy alongside with surgery, radiation and chemotherapy. Immunotherapy has shown great clinical efficacy in a number of hard-to-treat solid tumor malignancies.

It would be desirable to provide immunogenic molecules with affinity for cell markers relevant to diseases, such as cancer.

### SUMMARY

It is an object of the present disclosure to obviate or mitigate at least one disadvantage of previous immunotherapy approaches.

In one aspect, there is provided an isolated single domain antibody (sdAb), or a construct thereof comprising said sdAb, which sdAb binds specifically to human mesothelin (hMSLN).

In one aspect, the sdAb comprises i) a CDR1 amino acid sequence as set forth in SEQ ID NO: 86, 25, 4, 22, 1, 7, 10, 13, 16, 19, 28, 31, 34, 37, 40 or 43; ii) a CDR2 amino acid sequence as set forth in SEQ ID NO: 87, 26, 5, 23, 2, 8, 11, 14, 17, 20, 29, 32, 35, 38, 41, or 44; and iii) a CDR3 amino acid sequence as set forth in SEQ ID NO: 88, 27, 6, 24, 3, 9, 12, 15, 18, 21, 30, 33, 36, 39, 42, or 45. In one aspect, the sdAb may comprise CDR1, CDR2, and CDR3 amino acid sequences that are, as a group, at least 80%, at least 85%, or at least 90% identical to the respective CDR1, CDR2, and CDR3 amino acid sequences, as a group, selected from parts i)-iii).

In one aspect, there is provided an isolated single domain antibody (sdAb), or a construct thereof comprising said sdAb, which sdAb binds specifically to human mesothelin (hMSLN), the sdAb comprising:
a CDR1 amino acid sequence as set forth in SEQ ID NO:86, a CDR2 amino acid sequence as set forth in SEQ ID NO: 87, and a CDR3 amino acid sequence as set forth in SEQ ID NO: 88 (consensus from hMSLN-TP7-5 and hMSLN-TP7-56),
a CDR1 amino acid sequence as set forth in SEQ ID NO:4, a CDR2 amino acid sequence as set forth in SEQ ID NO: 5, and a CDR3 amino acid sequence as set forth in SEQ ID NO: 6 (from hMSLN-TP7-5),
a CDR1 amino acid sequence as set forth in SEQ ID NO: 25, a CDR2 amino acid sequence as set forth in SEQ ID NO: 26, and a CDR3 amino acid sequence as set forth in SEQ ID NO: 27 (from hMSLN-bioTP7-7),
a CDR1 amino acid sequence as set forth in SEQ ID NO: 22, a CDR2 amino acid sequence as set forth in SEQ ID NO: 23, and a CDR3 amino acid sequence as set forth in SEQ ID NO: 24 (from hMSLN-TP7-56),
a CDR1 amino acid sequence as set forth in SEQ ID NO: 1, a CDR2 amino acid sequence as set forth in SEQ ID NO: 2, and a CDR3 amino acid sequence as set forth in SEQ ID NO: 3 (from hMSLN-TP7-4),
a CDR1 amino acid sequence as set forth in SEQ ID NO: 7, a CDR2 amino acid sequence as set forth in SEQ ID NO: 8, and a CDR3 amino acid sequence as set forth in SEQ ID NO: 9 (from hMSLN-TP7-9),
a CDR1 amino acid sequence as set forth in SEQ ID NO: 10, a CDR2 amino acid sequence as set forth in SEQ ID NO: 11, and a CDR3 amino acid sequence as set forth in SEQ ID NO: 12 (from hMSLN-TP7-18),
a CDR1 amino acid sequence as set forth in SEQ ID NO: 13, a CDR2 amino acid sequence as set forth in SEQ ID NO: 14, and a CDR3 amino acid sequence as set forth in SEQ ID NO: 15 (from hMSLN-TP7-35),
a CDR1 amino acid sequence as set forth in SEQ ID NO: 16, a CDR2 amino acid sequence as set forth in SEQ ID NO: 17, and a CDR3 amino acid sequence as set forth in SEQ ID NO: 18 (from hMSLN-TP7-38A),
a CDR1 amino acid sequence as set forth in SEQ ID NO: 19, a CDR2 amino acid sequence as set forth in SEQ ID NO: 20, and a CDR3 amino acid sequence as set forth in SEQ ID NO: 21 (from hMSLN-TP7-38B),
a CDR1 amino acid sequence as set forth in SEQ ID NO: 28, a CDR2 amino acid sequence as set forth in SEQ ID NO: 29, and a CDR3 amino acid sequence as set forth in SEQ ID NO: 30 (from hMSLN-biomTP7-41),
a CDR1 amino acid sequence as set forth in SEQ ID NO: 31, a CDR2 amino acid sequence as set forth in SEQ ID NO: 32, and a CDR3 amino acid sequence as set forth in SEQ ID NO: 33 (from hMSLN-bioTP7-48),
a CDR1 amino acid sequence as set forth in SEQ ID NO: 34, a CDR2 amino acid sequence as set forth in SEQ ID NO: 35, and a CDR3 amino acid sequence as set forth in SEQ ID NO: 36 (from hMSLN-bioTP7-53),
a CDR1 amino acid sequence as set forth in SEQ ID NO: 37, a CDR2 amino acid sequence as set forth in SEQ ID NO: 38, and a CDR3 amino acid sequence as set forth in SEQ ID NO: 39 (from hMSLN-TP7-75A),
a CDR1 amino acid sequence as set forth in SEQ ID NO: 40, a CDR2 amino acid sequence as set forth in SEQ ID NO: 41, and a CDR3 amino acid sequence as set forth in SEQ ID NO: 42 (from hMSLN-TP7-75B), or
a CDR1 amino acid sequence as set forth in SEQ ID NO: 43, a CDR2 amino acid sequence as set forth in SEQ ID NO: 44, and a CDR3 amino acid sequence as set forth in SEQ ID NO: 45 (from hMSLN-mTP7-82).

In one aspect, the sdAb may comprise CDR1, CDR2, and CDR3 amino acid sequences that are, as a group, at least 80%, at least 85%, or at least 90% identical to the respective CDR1, CDR2, and CDR3 amino acid sequences, as a group from the sdAbs identified above.

It is noted that the CDR sequences of the antibodies herein termed hMSLN-TP7-38A and hMSLN-TP7-38B are identical to each other except for an Ala→Thr substitution at position 6 of CDR1 of hMSLN-TP7-38B as compared to hMSLN-TP7-38A. It is further noted that the CDR sequences of the antibodies herein termed hMSLN-TP7-75A and hMSLN-TP7-75B are identical to each other except for an Arg→Ser substitution at position 13 of CDR3 of hMSLN-TP7-75B as compared to hMSLN-TP7-75A.

In one aspect, there is provided an isolated single domain antibody (sdAb), or a construct thereof comprising said sdAb, which sdAb binds specifically to human mesothelin (hMSLN), the sdAb comprising: a CDR1 amino acid sequence as set forth in SEQ ID NO:86, a CDR2 amino acid sequence as set forth in SEQ ID NO: 87, and a CDR3 amino acid sequence as set forth in SEQ ID NO: 88 (consensus from hMSLN-TP7-5 and hMSLN-TP7-56).

In one aspect, there is provided an isolated single domain antibody (sdAb), or a construct thereof comprising said sdAb, which sdAb binds specifically to human mesothelin (hMSLN), the sdAb comprising: a CDR1 amino acid sequence as set forth in SEQ ID NO:4, a CDR2 amino acid sequence as set forth in SEQ ID NO: 5, and a CDR3 amino acid sequence as set forth in SEQ ID NO: 6 (from hMSLN-TP7-5).

In one aspect, there is provided an isolated single domain antibody (sdAb), or a construct thereof comprising said sdAb, which sdAb binds specifically to human mesothelin (hMSLN), the sdAb comprising: a CDR1 amino acid sequence as set forth in SEQ ID NO: 25, a CDR2 amino acid sequence as set forth in SEQ ID NO: 26, and a CDR3 amino acid sequence as set forth in SEQ ID NO: 27 (from hMSLN-bioTP7-7).

In one aspect, there is provided an isolated single domain antibody (sdAb), or a construct thereof comprising said sdAb, which sdAb binds specifically to human mesothelin (hMSLN), the sdAb comprising: a CDR1 amino acid sequence as set forth in SEQ ID NO: 22, a CDR2 amino acid sequence as set forth in SEQ ID NO: 23, and a CDR3 amino acid sequence as set forth in SEQ ID NO: 24 (from hMSLN-TP7-56).

In one aspect, there is provided a sdAb or construct thereof, as described herein, wherein the sdAb is humanized.

In one aspect, there is provided a sdAb or construct thereof, as described herein, wherein the sdAb has a binding affinity (K_{D}) for hMSLN of 5.8×10⁻⁸ M or less, more preferably 3×10⁻⁸ M or less, more preferably 2×10⁻⁹ M or less, more preferably 6×10⁻¹⁰ M or less, more preferably 5.79×10⁻¹¹ M or less.

In one aspect, there is provided a sdAb or construct thereof, as described herein, wherein the construct is a multivalent antibody comprising: a first antigen-binding portion comprising the sdAb, and a second antigen-binding portion.

In one aspect, the second antigen-binding portion is selected from the group consisting of an scFv, a second sdAb, an aptamer, a protein receptor, or a cytokine. In one aspect, the second antigen-binding portion binds specifically to a cell-surface marker of an immune cell. The cell surface marker may optionally be selected from the group consisting of a T-cell marker, NK-cell marker, or a T- and NK-cell marker. In one aspect, the T-cell marker comprises human CD3. In another aspect, the NK-cell marker is human NKp30.

In one aspect, the multivalent antibody is a dimeric immune-cell engager, said multivalent antibody further comprising a human Fc domain.

In one aspect, the sdAb construct is a multivalent antibody encoded by SEQ ID NO: 82 or SEQ ID NO: 83, or an amino acid sequence that is at least 80%, at last 85%, at least 90%, or at least 95% identical thereto.

In one aspect, the multivalent antibody comprises a third antigen-binding portion that binds to an antigen target that may or may not be distinct from hMSLN. In one aspect, the third antigen-binding portion may be selected to bind to human serum albumin, to extend serum half-life.

In one aspect, there is provided a sdAb or construct thereof, as described herein, wherein the sdAb construct is a chimeric antibody receptor (CAR), which specifically binds to human mesothelin (hMSLN).

In one aspect, the CAR comprises, in an N-terminal to C-terminal direction: the sdAb, a polypeptide hinge, a transmembrane domain, and a cytoplasmic domain comprising at least one signaling domain, preferably wherein the cytoplasmic domain further comprises a co-stimulatory domain.

In one aspect, the CAR comprises the amino acid sequence of any one of SEQ ID NOs: 61 to 66 or an amino acid sequence that is at least 80%, at last 85%, at least 90%, or at least 95% identical thereto.

In one aspect, the CAR further comprises a second binding domain.

In one aspect, there is provided a nucleic acid molecule encoding any of the sdAbs or constructs thereof described herein. In one aspect, there is provided a recombinant viral particle comprising said nucleic acid molecule. In one aspect, there is provided a cell comprising said nucleic acid molecule.

In one aspect, there is provided an engineered cell expressing at the cell surface membrane the CAR as described herein. In one aspect, said engineered cell is an immune cell, preferably a leukocyte, more preferably a T-cell, a monocyte, a macrophage, or a neutrophil. In one aspect, said engineered cell is an induced pluripotent stem cell (iPSC), or differentiated cell product derived thereof. In one aspect, the engineered cell is an immune cell derived from T-lymphocytes.

In one aspect there is provided a use, for the treatment of a cancer, of the sdAb or construct thereof as described herein;

In one aspect there is provided a use, for the treatment of a cancer, of the nucleic acid molecule as described herein.

In one aspect there is provided a use, for the treatment of a cancer, of the recombinant viral particle as described herein.

In one aspect there is provided a use, for the treatment of a cancer, of the cell as described herein.

In one aspect there is provided a use, for the treatment of a cancer, of the engineered cell as described herein.

In one aspect, there is provided a method of treating a cancer in subject comprising administering to the subject: the sdAb or construct thereof as described herein; the nucleic acid molecule as described herein; the recombinant viral particle as described herein; the cell as described herein; or the engineered cell as described herein.

In one aspect, the cancer comprises cells that aberrantly over-express MSLN compared to corresponding healthy cells.

In one aspect, there is provided a VHH single domain antibody (sdAb)), or a construct thereof comprising said sdAb, which sdAb that competes for specific binding to MSLN with one of the isolated sdAbs described above (a “competing sdAb”).

In one aspect, there is provided a recombinant polypeptide comprising an sdAb as defined herein.

In one aspect, there is provided the sdAb defined herein fused to a human Fc (fragment crystallizable region) (termed a “V_{H}H:Fc fusion”).

In a further aspect, the present disclosure provides anti-MSLN sdAb as defined herein linked to a cargo molecule.

In one aspect, there is provided a recombinant nucleic acid molecule encoding an sdAb.

In one aspect, there is provided a composition comprising the sdAb as defined herein, or a polypeptide comprising such an sdAb; together with an acceptable excipient, diluent or carrier.

In one aspect, there is provided a use of the sdAb as defined herein, or of an antibody comprising one or more V_{H}H or V_{H}H:Fc fusion comprising the sdAb as defined herein, for treatment of solid cancer or a hematological malignancy.

In one aspect, there is provided a use of the sdAb as defined herein, or of an antibody comprising one or more VAH or V_{H}H:Fc fusion comprising the sdAb as defined herein, for preparation of a medicament for treatment of solid cancer or a hematological malignancy.

In one aspect, there is provided the sdAb as defined herein, or of an antibody comprising one or more V_{H}H or V_{H}H:Fc fusion comprising the sdAb as defined herein, for use in treatment of solid cancer or a hematological malignancy.

In one aspect, there is provided a method of treating solid cancer or a hematological malignancy in subject comprising administering to the subject the sdAb as defined herein, or of an antibody comprising one or more V_{H}H or V_{H}H:Fc fusion comprising the sdAb as defined herein.

In one aspect, there is provided a use of the sdAb as defined herein, or of an antibody comprising one or more V_{H}H or V_{H}H:Fc fusion comprising the sdAb as defined herein, for delivery of a lipid nanoparticle (LNP) to a cell.

In one aspect, there is provided a use of the sdAb as defined herein, or of an antibody comprising one or more V_{H}H or V_{H}H:Fc fusion comprising the sdAb as defined herein, for preparation of a medicament for delivery of a lipid nanoparticle (LNP) to a cell.

In one aspect, there is provided the sdAb as defined herein, or of an antibody comprising one or more V_{H}H or V_{H}H:Fc fusion comprising the sdAb as defined herein, for use in delivery of a lipid nanoparticle (LNP) to a cell.

In one aspect, there is provided a method of delivering an LNP to a cell comprising contacting the cell with the sdAb as defined herein, or of an antibody comprising one or more V_{H}H or V_{H}H:Fc fusion comprising the sdAb as defined herein.

In one aspect, there is provided a multivalent antibody comprising an sdAb as defined above.

In aspect, there is provided a recombinant nucleic acid molecule encoding the multivalent antibody as defined herein.

In one aspect, there is provided a composition comprising a multivalent antibody as defined herein; together with an acceptable excipient, diluent or carrier.

In one aspect, there is provided a use of the multivalent antibody as defined herein for treatment of solid cancer or a hematological malignancy.

In one aspect, there is provided a use of the multivalent antibody as defined herein for preparation of a medicament for treatment of a cancer or a hematological malignancy.

In one aspect, there is provided the multivalent antibody as defined herein for use in treatment of a cancer or a hematological malignancy.

In one aspect, there is provided a method of treating a cancer or a hematological malignancy in subject comprising administering to the subject the multivalent antibody as defined herein.

In one aspect, there is provided a chimeric antibody receptor (CAR), which binds to human MSLN, comprising the V_{H}H sdAb as defined herein.

In one aspect, there is provided a recombinant nucleic acid molecule (either DNA or RNA) encoding the CAR as defined herein.

In one aspect, there is provided a vector comprising the recombinant nucleic acid molecule as defined herein.

In one aspect, there is provided a recombinant viral particle comprising the recombinant nucleic acid as defined herein.

In one aspect, there is provided a cell comprising the recombinant nucleic acid molecule as defined herein.

In one aspect, there is provided an engineered cell expressing at the cell surface membrane the CAR as defined herein.

In one aspect, there is providing a use of the nucleic acid, vector, or viral particle as described herein for preparation of cells for a CAR application.

In one aspect, there is providing a method of preparing cells for a CAR application comprising contacting a cell with the viral particle as described herein.

In one aspect, there is providing a method of preparing cells for a CAR application comprising introducing into a cell the nucleic acid or vector as described herein.

In one aspect, there is provided a use of the CAR or of the engineered cell as described herein for treatment of a solid cancer.

In one aspect, there is provided a use of the CAR or of the engineered cell as described herein for preparation of a medicament treatment of a solid cancer.

In one aspect, there is provided the CAR or the engineered cell as described herein for use in treatment of a solid cancer.

In one aspect there is provided a method of treating a solid cancer in a subject, comprising administering to the subject the engineered cell as defined herein.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 depicts the processing of human MSLN (known also as Pre-pro-megakaryocyte-potentiating factor or CAK1 antigen) molecule from its precursor protein on the cell surface.

FIG. 2A depicts a SDS-PAGE of IMAC-purified human isoform I MSLN extracellular domain chain (309aa+10×His-tag) under reducing and non-reducing conditions.

FIG. 2B depicts mass spectrometry data demonstrating that the MSLN protein mass is consistent with the expected molecular weight (36.3 kDa) and is largely intact. The protein was treated with PNGaseF prior to mass spectrometry analysis, but some glycosylation remains. The glycosylated nature of the proteins is also reflected on the protein gel.

FIG. 3 depicts the llama polyclonal immune response from a pre-immune test bleed (day 0) and the final bleed (7 days post 5th immunization) against hMSLN protein. The mouse recombinant MSLN (mMSLN) (~58% identity with hMSLN) produced in a similar method at the NRC-HHT was used in ELISA and the results showed that there is a weaker immune response against the mMSLN.

FIG. 4 depicts the phage ELISA on individual VHH clones (37 in total) obtained after four rounds of panning. As shown, both hMSLN and mMSLN were used in the ELISA and some VHH clones show some levels of cross-reactivity with both proteins. Note that similar phage ELISA data were obtained from passive absorption panning against human and mouse MSLN. Here we show only the phage ELISA of biotinylated MSLN panning against mouse and human MSLN with some clones repeated (bioTP7-7) as determined by sequencing.

FIG. 5 depicts the SDS-PAGE of 11 anti-MSLN VHH antibodies expressed in BL21 (DE3) E. coli and purified by IMAC. The protein data from the two VHHs (biomTP7-41 and bioTP48) are not included in this figure but their pattern of VHH expression is very similar to those VHHs shown here.

FIG. 6 depicts a partial amino acid sequence alignment of 13 unique VHHs parsed according to the IMGT numbering system.

FIG. 7 depicts the SPR binding and epitope binning of anti-hMSLN VHHs to the immobilized hMSLN on a CM5 microchip through amine-coupling.

FIGS. 8A to 81 depict hMSLN cell binding of the selected VHHs by Mirrorball. MSLN-positive (H292) and MSLN-negative (H1581) cells were used. As positive control, a commercial anti-MSLN mAb was used (FIG. 8A). FIGS. 8B to 81 depict: TP7-4, TP7-5, TP7-9, TP7-18, TP7-35. TP7-38, TP7-56, and TP7-75, respectively. The VHHs: bioTP7-7, biomTP7-41, bioTP7-48, bioTP7-53, mTP7-82 were not used in this study.

FIG. 9 depicts the results of a cell binding assessment of purified single domain antibodies measured using flow cytometry.

FIG. 10 depicts the results of CAR-Jurkat assay wherein Jurkat cells were electroporated with varying CAR plasmids and CAR-J cells (Jurkat cells transiently expressing the CAR) cultured alone or in co-culture with MSLN-positive SKOV3 cells.

FIG. 11 depicts the results of CAR-T tonic activation assay wherein primary donor blood derived T cells were transduced with varying CAR constructs and examined for target-independent expansion.

FIG. 12 depicts the results of CAR-T co-culture assay performed over 6 weeks. Healthy human donor blood derived T cells transduced with varying MSLN-single domain antibody-based CAR constructs or unmodified donor T cells (Mock) were co-cultured with target cells expressing MSLN antigen (H292 cells) or devoid of MSLN antigen (Raji cells). Target cells were also expressing nuclear-restricted mKate2. For this, target cells were transduced with a third generation HIV-based VSV-G pseudotyped lentivirus encoding a nuclear-localized mKate2 (Sartorius, Essen BioScience, Bohemia, NJ, USA). NucLight™ positive cells were obtained by selection with puromycin. CAR-T mediated target-specific tumor cell growth suppression was evaluated using a Sartorius IncuCyte® S3 (Essen Bioscience) and automated cell counting of red target cells was performed using IncuCyte® analysis software.

FIG. 13 depicts the results of CAR-T co-culture assay performed over 6 weeks. Healthy human donor blood derived T cells transduced with varying MSLN-single domain antibody-based CAR constructs or unmodified donor T cells (Mock) were co-cultured with target cells expressing MSLN antigen (H292 cells) or devoid of MSLN antigen (Raji cells). The CAR-T constructs contained a P2A ribosomal self-skipping sequence separating the CAR from an EGFP marker, thereby allowing fluorescent detection of CAR-expressing cells. Target-specific CAR-T cell proliferation was evaluated using a Sartorius IncuCyte® S3 (Essen Bioscience) and automated cell counting of green CAR-T cells was performed using IncuCyte® analysis software.

FIG. 14 depicts a design for a tandem CAR targeting multiple antigens through linking both binding domains to a single CAR molecule.

FIG. 15 depicts the results of testing of various exemplary tandem-CAR molecules combining mesothelin targeting agents with BCMA or CD22 targeting agents, wherein plasmids encoding various CARs or tandem CARs were examined via CAR-J assay similarly as described elsewhere to test their response to various target cells.

FIG. 16 depicts the results of testing of various exemplary tandem-CAR molecules combining mesothelin targeting agents with an EGFR targeting sdAb, wherein plasmids encoding single target MSLN or EGFR CARs or tandem CARs wherein EGFR and MSLN sdAbs were combined in either orientation then were examined via CAR-J assay similarly as described elsewhere to test their response to various target cells.

FIG. 17 depicts result of tumor burden in mice that were inoculated with H292 tumor cells and treated with various CAR-T cells

FIG. 18 depicts the proportion of surviving animals in each treatment group throughout the course of the experiment.

FIG. 19 depicts the tumour volume measurements for NSG mice injected subcutaneously with H292 human lung tumour cells and treated with marginal doses of various CAR-T cells.

FIG. 20 depicts the molecular structure of MSLN-specific single domain antibody bi-specific T cell engager proteins.

FIG. 21 depicts combined data from FIGS. 21A and 21B.

FIG. 22 depicts Jurkat activity testing for a purified MSLN-bispecific T cell engager tested as described elsewhere.

FIG. 23 depicts a dimeric human-Fc fused form of a MSLN-VHH-CD3scFv-huFc bispecific engager.

FIG. 24 depicts the results of functional testing of a purified dimeric MSLN-VHH-CD3scFv-huFc molecule, which shows activity in the attomolar (10⁻¹⁸ mols/L) range.

FIG. 25 shows representative images demonstrating potent T-cell mediated killing of MSLN+ H292 Lung Cancer cells at attomolar (10⁻¹⁸) concentration, which equates to only 10 bispecific antibody molecules per target cell in culture.

FIG. 26 depicts the construct design for a proof-of-concept MSLN-VHH bispecific killer engager molecule which will simultaneously engage both NK cells and target cells

FIG. 27 shows representative images demonstrating NK-cell mediated killing of MSLN+ H292 Lung Cancer cells at femtomolar concentration

FIG. 28 depicts the assay concept for testing tissue and tumour binding of MSLN-specific sdAb molecules.

FIG. 29 depicts control immunohistochemical staining of a human tumor and corresponding healthy tissue array, wherein tissue was subjected to staining in the absence of any primary antibody

FIG. 30 depicts control immunohistochemical staining of a human tumor and corresponding healthy tissue array, wherein tissue was stained with an irrelevant primary antibody (non-human protein specific).

FIG. 31 depicts immunohistochemical staining of a human tumor and corresponding healthy tissue array, wherein tissue was stained with the TP7-56 single domain antibody.

FIG. 32 depicts immunohistochemical staining of a human tumor and corresponding healthy tissue array, wherein tissue was stained with the TP7-5 single domain antibody.

### DETAILED DESCRIPTION

The present disclosure provides anti-MSLN single domain antibodies (sdAb) or VHHs prepared by immunizing a llama with the ecto-domain of human MSLN (hMSLN). By constructing a library of the heavy chain repertoire generated, VHH antibodies specific to the immunogen were isolated. The 15 unique example antibodies initially produced comprise CDR1, CDR2, and CDR3 sequences corresponding, respectively to SEQ NOs: 1-3, 4-6, 7-9, 10-12, 13-15, 16-18, 19-21, 22-24, 25-27, 28-30, 31-33, 34-36, 37-39, 40-42, 43-45; and related sequences. Also provided are multivalent antibodies comprising any one of the sdAbs, including bispecific T-cell engagers, bispecific killer cell engagers (BiKEs), and trispecific killer cell engagers (TrikEs). Also described are chimeric antigen receptors (CARs) for CAR-T and CAR-NK therapy comprising any one or more of the aforementioned sdAbs. Uses of these molecules in the treatment of cancer or autoimmune diseases are also described, in particular hematological malignancies, such as multiple myeloma.

### Single Domain Antibodies and Polypeptides Comprising Them

Single domain antibodies (sdAbs), also known as nanobodies are derived from the heavy-chain antibodies found in Camelidae species (such as camel, llama, dromedary, alpaca and guanaco) using molecular biology techniques, which are also known as V_{H}H fragments (herein also termed “V_{H}H” or “VHH”). Similar antibody domains including V_{NAR} fragments derived from heavy chain antibodies found in cartilaginous fish, such as sharks. sdAbs could also been generated from a heavy chain/light chain of conventional immunoglobulin G (IgGs) by engineering techniques followed by affinity maturations, or alternatively, from an immunized transgenic mouse or rat carrying the camelid heavy chain or humanized camelid gene loci.

Despite their small (about 10× smaller than mAbs), sdAbs have comparable affinity and specificity, and quite stable under extreme pH, high temperature and proteolytic conditions that can be problematic for conventional antibodies and fragments thereof (e.g., Fab, scFv). Furthermore, V_{H}Hs could be expressed in simple prokaryotic and eukaryotic organisms (e.g., bacteria and yeast) in up to gram quantities and in properly folded/functional formats. The unique feature of a portion of VHH antibodies generated in Camelidae species is that they recognize cryptic or hidden epitopes such as enzyme active sites or cavities on virus surface proteins which are otherwise inaccessible to larger conventional antibodies and antibody fragments. This will place camelid sdAbs in a unique position for the development of enzyme inhibitor or viral neutralizer reagents.

“CDRs” or “complementarity-determining regions” are the portion of the variable chains in immunoglobulins that collectively constitute the paratope, and thereby impart binding specificity and affinity to the antibody. As used here, the term refers to CDRs mapped in sdAbs according to the standards or conventions set by IMGT™ (international ImMunoGeneTics information system).

The antibodies described herein have been raised to the recombinant extracellular domain (ECD) of human MSLN isoform 1. An example mRNA sequence for this isoform may be found in GenBank entry AY743922.1, wherein encoded amino acids 296 to 606 correspond to the processed ECD (see also UniProt entry Q13421).

In one aspect, there is provided an isolated single domain antibody (sdAb) which binds specifically to human MSLN (hMSLN), the sdAb comprising:

### A)

a CDR1 amino acid sequence as set forth in SEQ ID NO:86, a CDR2 amino acid sequence as set forth in SEQ ID NO: 87, and a CDR3 amino acid sequence as set forth in SEQ ID NO: 88 (consensus from hMSLN-TP7-5 and hMSLN-TP7-56), a CDR1 amino acid sequence as set forth in SEQ ID NO: 1, a CDR2 amino
acid sequence as set forth in SEQ ID NO: 2, and a CDR3 amino acid sequence as set forth in SEQ ID NO: 3 (hMSLN-TP7-4),
a CDR1 amino acid sequence as set forth in SEQ ID NO: 4, a CDR2 amino acid sequence as set forth in SEQ ID NO: 5, and a CDR3 amino acid sequence as set forth in SEQ ID NO: 6 (hMSLN-TP7-5),
a CDR1 amino acid sequence as set forth in SEQ ID NO: 7, a CDR2 amino acid sequence as set forth in SEQ ID NO: 8, and a CDR3 amino acid sequence as set forth in SEQ ID NO: 9 (hMSLN-TP7-9),
a CDR1 amino acid sequence as set forth in SEQ ID NO: 10, a CDR2 amino acid sequence as set forth in SEQ ID NO: 11, and a CDR3 amino acid sequence as set forth in SEQ ID NO: 12 (hMSLN-TP7-18),
a CDR1 amino acid sequence as set forth in SEQ ID NO: 13, a CDR2 amino acid sequence as set forth in SEQ ID NO: 14, and a CDR3 amino acid sequence as set forth in SEQ ID NO: 15 (hMSLN-TP7-35),
a CDR1 amino acid sequence as set forth in SEQ ID NO: 16, a CDR2 amino acid sequence as set forth in SEQ ID NO: 17, and a CDR3 amino acid sequence as set forth in SEQ ID NO: 18 (hMSLN-TP7-38A),
a CDR1 amino acid sequence as set forth in SEQ ID NO: 19, a CDR2 amino acid sequence as set forth in SEQ ID NO: 20, and a CDR3 amino acid sequence as set forth in SEQ ID NO: 21 (hMSLN-TP7-38B),
a CDR1 amino acid sequence as set forth in SEQ ID NO: 22, a CDR2 amino acid sequence as set forth in SEQ ID NO: 23, and a CDR3 amino acid sequence as set forth in SEQ ID NO: 24 (hMSLN-TP7-56),
a CDR1 amino acid sequence as set forth in SEQ ID NO: 25, a CDR2 amino acid sequence as set forth in SEQ ID NO: 26, and a CDR3 amino acid sequence as set forth in SEQ ID NO: 27 (hMSLN-bioTP7-7),
a CDR1 amino acid sequence as set forth in SEQ ID NO: 28, a CDR2 amino acid sequence as set forth in SEQ ID NO: 29, and a CDR3 amino acid sequence as set forth in SEQ ID NO: 30 (hMSLN-biomTP7-41)
a CDR1 amino acid sequence as set forth in SEQ ID NO: 31, a CDR2 amino acid sequence as set forth in SEQ ID NO: 32, and a CDR3 amino acid sequence as set forth in SEQ ID NO: 33 (hMSLN-bioTP7-48),
a CDR1 amino acid sequence as set forth in SEQ ID NO: 34, a CDR2 amino acid sequence as set forth in SEQ ID NO: 35, and a CDR3 amino acid sequence as set forth in SEQ ID NO: 36 (hMSLN-bioTP7-53),
a CDR1 amino acid sequence as set forth in SEQ ID NO: 37, a CDR2 amino acid sequence as set forth in SEQ ID NO: 38, and a CDR3 amino acid sequence as set forth in SEQ ID NO: 39 (hMSLN-TP7-75A),
a CDR1 amino acid sequence as set forth in SEQ ID NO: 40, a CDR2 amino acid sequence as set forth in SEQ ID NO: 41, and a CDR3 amino acid sequence as set forth in SEQ ID NO: 42 (hMSLN-TP7-75B), or
a CDR1 amino acid sequence as set forth in SEQ ID NO: 43, a CDR2 amino acid sequence as set forth in SEQ ID NO: 44, and a CDR3 amino acid sequence as set forth in SEQ ID NO: 45 (hMSLN-mTP7-82), or

### B)

CDR1, CDR2, and CDR3 amino acid sequences that are at least 80% identical to the CDR1, CDR2, and CDR3 sequences defined in any one of part A).

In one embodiment, in B) the CDR 1 CDR2, and CDR3 amino acid sequences are at least 90% identical to the CDR1, CDR2, and CDR3 sequences defined in any one of part A). In one embodiment, in B) the CDR 1 CDR2, and CDR3 amino acid sequences are at least 95% identical to the CDR1, CDR2, and CDR3 sequences defined in any one of part A). In one embodiment, in B) the CDR 1 CDR2, and CDR3 amino acid sequences have at most three substitutions compared to the CDR1, CDR2, and CDR3 sequences defined in any one of part A). In one embodiment, in B) the CDR 1 CDR2, and CDR3 amino acid sequences have at most two substitutions compared to the CDR1, CDR2, and CDR3 sequences defined in any one of part A). In one embodiment, in B) the CDR 1 CDR2, and CDR3 amino acid sequences have at most one substitution compared to the CDR1, CDR2, and CDR3 sequences defined in any one of part A). In some embodiment, sequence differences vs. the sequences set forth in A) are conservative sequence substitutions.

The term “conservative amino acid substitutions” which is known in the art is defined herein as follows, with conservative substitutable candidate amino acids showing in parentheses: Ala (Gly, Ser); Arg (Gly, Gln); Asn (Gln; His); Asp (Glu); Cys (Ser); Gln (Asn, Lys); Glu (Asp); Gly (Ala, Pro); His (Asn; Gln); Ile (Leu; Val); Leu (Ile; Val); Lys (Arg; Gln); Met (Leu, Ile); Phe (Met, Leu, Tyr); Ser (Thr; Gly); Thr (Ser; Val); Trp (Tyr); Tyr (Trp; Phe); Val (Ile; Leu).

Sequence variants according to certain embodiments are intended to encompass molecules in which binding affinity and/or specificity is substantially unaltered vs. the parent molecule from which it is derived. Such parameters can be readily tested, e.g., using techniques described herein and techniques known in the art. Such embodiments may encompass sequence substitutions, insertions, or deletions.

These embodiments are intended to encompass, inter alia, embodiments in which molecules recovered following mutagenization/diversification of CDR2, and screening for variant molecules that bind to MSLN and/or cross-compete for binding to MSLN with the parent molecule from which they are defined. As above, a library could be screened or individual candidate molecules could be tested.

In one embodiment, sdAb comprises A) the amino acid sequence of any one of SEQ ID NO: 46 to 60, or B) an amino acid sequence that is at least 80% identical to any one of SEQ ID NO: 46 to 60 across the full length thereof. In one embodiment, the amino acid sequence of B) is at least 85% identical across the full length therefore to one of the amino acid sequences of A). In one embodiment, the amino acid sequence of B) is at least 90% identical across the full length therefore to one of the amino acid sequences of A). In one embodiment, the amino acid sequence of B) is at least 95% identical across the full length therefore to one of the amino acid sequences of A). In one embodiment, the amino acid sequence of B) is at least 98% identical across the full length therefore to one of the amino acid sequences of A). In one embodiment, the amino acid sequence of B) is at least 98% identical across the full length therefore to one of the amino acid sequences of A). In some of these embodiments, sequences differences vs. sequences of A) are outside the CDR sequences.

In one embodiment of the above, the CDR1, CDR2, and CDR3 are defined with respect to the IMGT™ numbering system. It is to be appreciated that CDR sequences could be defined by other conventions, such as the Kabat, Chothia, or EU numbering systems.

In one embodiment, the CDR1, CDR2, and CDR3 are defined according to the Kabat, the Chothia convention, or the EU numbering convention.

In one embodiment, the sdAb comprises any one of SEQ ID NOs: 46 to 60.

In some embodiments, the sdAb comprises any one of SEQ ID NOs: 46 to 60, wherein the residue at position 3 (if not already Q) is substituted with Q. In some embodiments, the sdAb comprises any one of SEQ ID NOs: 46 to 60, wherein the residue at position 3 (if not already K) is substituted with K.

In some embodiments, the sdAb comprises an amino acid sequence according to any one of SEQ ID NOs: 46 to 60, wherein the residue at position 5 (if not already V) is substituted with V. In some embodiments, the sdAb comprises any one of SEQ ID NOs: 46 to 60, wherein the residue at position 5 (if not already E) is substituted with E.

In some embodiments, the sdAb comprises any one of SEQ ID NOs: 46 to 60, wherein the residue at position 1 (if not already Q) is substituted with Q. In some embodiments, the sdAb comprises any one of SEQ ID NOs: 46 to 60, wherein the residue at position 1 (if not already E) is substituted with E. In some embodiments, the sdAb comprises any one of SEQ ID NOs: 46 to 60, wherein the residue at position 1 (if not already D) is substituted with D.

In some embodiments, the sdAb comprises any one of SEQ ID NOs: 46 to 60, wherein the first five N-terminal residues (if not already QVQLV) are substituted for QVQLV. In some embodiments, the sdAb comprises any one of SEQ ID NOs: 46 to 60, wherein the first five N-terminal residues (if not already QVKLE) are substituted for QVKLE. In some embodiments, the sdAb comprises any one of SEQ ID NOs: 46 to 60, wherein the first five N-terminal residues (if not already DVQLV) are substituted for DVQLV. In some embodiments, the sdAb comprises any one of SEQ ID NOs: 46 to 60, wherein the first five N-terminal residues (if not already EVQLV) are substituted for EVQLV.

In one embodiment, the sdAb is a Camelidae V_{H}H sdAb.

In one embodiment, the sdAb is a llama V_{H}H sdAb.

In one embodiment, the sdAb is humanized V_{H}H sdAb.

By the term “humanized” as used herein is meant mutated so that immunogenicity upon administration in human patients is minor or nonexistent. Humanizing a polypeptide, according to the present invention, comprises a step of replacing one or more of the Camelidae amino acids by their human counterpart as found in the human consensus sequence, without that polypeptide losing its typical character, i.e. the humanization does not significantly affect the antigen binding capacity of the resulting polypeptide. A humanized antibody can be produced using a variety of techniques known in the art, including but not limited to, CDR-grafting, veneering or resurfacing, chain shuffling, etc.

In one embodiment, the sdAb has an affinity for human MSLN of 5.8×10⁻⁸ M or less. In one embodiment, the sdAb has an affinity for human MSLN of 3×10⁻⁸ M or less. In one embodiment, the sdAb has an affinity for human MSLN of 2×10⁻⁹ M or less. In one embodiment, the sdAb has an affinity for human MSLN of 6×10⁻¹⁰ M or less. In one embodiment, the sdAb has an affinity for human MSLN of 5.79×10⁻¹¹ M or less. Binding affinity can be determined, e.g., according to assays described herein.

In one aspect, there is provided a V_{H}H single domain antibody (sdAb) that competes for specific binding to MSLN with one of the isolated sdAbs described above (a “competing sdAb”). A competing sdAb may be identified by a method that comprises a binding assay which assesses whether or not a test antibody is able to cross-compete with a known antibody of the invention for a binding site on the target molecule. For example, the antibodies described hereinabove may be used as reference antibodies. Methods for carrying out competitive binding assays are well known in the art. For example, they may involve contacting together a known antibody of the invention and a target molecule under conditions which the antibody can bind to the target molecule. The antibody/target complex may then be contacted with a test antibody and the extent to which the test antibody is able to displace the antibody of the invention from antibody/target complexes may be assessed. An alternative method may involve contacting a test antibody with a target molecule under conditions that allow for antibody binding, then adding an antibody of the invention that is capable of binding that target molecule and assessing the extent to which the antibody of the invention is able to displace the test antibody from antibody/target complexes. Such antibodies may be identified by generating new sdAbs to MSLN and screening the resulting library for cross-competition. Alternatively, one of the antibodies described herein may serve as a starting point for diversification, library generation, and screening. A further alternative could involve testing individual variants of an antibody described herein.

In one embodiment, the sdAb does additionally bind specifically to mouse mesothelin (mMSLN). In one embodiment, this is determined according to the SPR assay described herein (see Example 2). See, for example, antibodies TP7-4, mTP7-82 and bioTP7-7 as described herein (Table 3B).

In one embodiment, the sdAb does not bind to mouse mesothelin (mMSLN).

In one embodiment, this is determined according to the SPR assay described herein (see Example 2). See, for example, antibodies TP7-5, TP7-9, TP7-18, TP7-35, TP7-38, TP7-56, TP7-75, biomTP7-41, bioTP4-78, and bioTP7-53.

For embodiments described, specific embodiments are contemplated comprising the full-length sdAb as herein described.

Table 1 lists full-length sequences for various sdAb disclosed herein according to some embodiments. CDR1, CDR2, and CDR3 sequences are underlined. CDR identification and numbering used herein is according to the IMGT™ convention.

TABLE 1
VHH Sequences

| | Name | Sequence (CDRs Underlined) |
|---|---|---|
| 1 | hMSLN- | QVQLVGSGGGLVRAGGSLRLSCAGSGLTFGSYAMAWYRQAPGKQRELVASISSRGVTN |
| | TP7-4 | YADSVEGRFTISRDNAKSTVYLQMNDLKPEDAAVYYCNAKGPFMLREYDYWGQGTQVT |
| | | VSS |
| 2 | hMSLN- | QVKLEESGGGLVQAGGSLRISCAASGRSISDYFMGWFRQTPGKEREFVAAISWSGGTA |
| | TP7-5 | YYADSVK |
| | | GRFTISRDNAKNTGYLQMNSLKPEDTAVYFCAARLSYDSRTSLQNSYDYNYWGQGTQV |
| | | TVSS |
| 3 | hMSLN- | DVQLVDSGGGLVQAGGSLRLSCAASGRTVSTYNMAWFRQAPGKEREIVAVINWTGGST |
| | TP7-9 | YYAESVKGRFTISGDNPKNTVYLQMNSLKPEDTAVYYCAATRASWTENTYWGQGTQVT |
| | | VSS |
| 4 | hMSLN- | EVQLVESGGRLVQAGGSLRLSCVASGRAFKWWTMGWFRQAPGKEREFVASISWSDGST |
| | TP7-18 | TYGDSVKGRFAISRDNNKNTVYLQMNSLTPEDTAVYYCARYGKDLSGRHSKNVQDYDY |
| | | WGQGTQVTVSS |
| 5 | hMSLN- | QVQLVDSGGGLVQPGGSLRLSCTTSASTFRFFAMHWYRQAPGKERELVAVISRDGDAE |
| | TP7-35 | YVDSVKGRFTISRDNAKNAVYLQMNSLKPEDTAVYYCHTRILSDHYWGQGTQVTVSS |
| 6 | hMSLN- | QVKLEESGGGLVQPGGSLRLSCAASGRTVSAYNMAWFRQAPGKEREIVAVINWTGGGT |
| | TP7-38A | YYAESVKGRFTISRDNPKNTVYLQMNSLKPEDTAVYYCAATRTSWTENTYWGQGTQVT |
| | | VSS |
| 7 | hMSLN- | QVQLVESGGGLVQPGGSLRLSCAASGRTVSTYNMAWFRQAPGKEREIVAVINWTGGGT |
| | TP7-38B | YYAESVKGRFTISRDNPKNTVYLQMNSLKPEDTAVYYCAATRTSWTENTYWGQGTQVT |
| | | VSS |
| 8 | hMSLN- | QVQLVESGGGLVQAGGSLRISCVASGRDFSSHFMGWSRQAPGKEREFVAAISWSGGTT |
| | TP7-56 | YYADSVKGRFAISRDNDKTTVYLQMNSLKPEDSAVYYCAARRTYDSRTSLRNSYDYPY |
| | | WGQGTQVTVSS |
| 9 | hMSLN- | QVQLVESGGGLVQAGGSLRLSCAASGRTVSTYNMAWFRQAPGKEREIVAVINWTGGST |
| | bioTP7-7 | YYAESVKGRFTISRDNPKNTVYLQMNSLKPEDTAVYYCAATRTSWTENTYWGQGTQVT |
| | | VSS |
| 10 | hMSLN- | QVQLVESGGGLVQPGGSLRLSCTASGIVRASGMAWYRQAPGKQRELVASITTGGETTW |
| | biomTP7- | YADSVKGRFTISIDNAKMMLYLQMNALKPEDTAVYLCNQRSYWGQGTQVTVSS |
| | 41 | |
| 11 | hMSLN- | QVKLEESGGGLVQPGGSLRLSCTASGSIWSASGLTWYRQRPGNERELVASITTGGETT |
| | bioTP7- | WYADSVKGRFTISISRDNGKMITYLQMNSLKPEDTAVYLCNSRSYWGQGTQVTVSS |
| | 48 | |
| 12 | hMSLN- | QVQLVESGGGLVQPGGSLRLACAASGGIGSLSNVRWYRQGPGKEREYVAGITGQSTTY |
| | bioTP7- | YADSVKGRFTISRDNVKNTVDLQMNSLKPEDTAVYICNVATLRNEYWGQGTQVTVSS |
| | 53 | |
| 13 | hMSLN- | QVQLVESGGGLVQAGGSLSLSCAASGRTFSASAMAWFRQAPGTDREFVAAVAWGGRDT |
| | TP7-75A | HYADSVKGRFTISMNNAKNTVVLQMNSLKPEDTAVYYCAAGQRGWSHSTSRDYDYWGQ |
| | | GTQVTVSS |
| 14 | hMSLN- | QVQLVESGGGLVQAGGSLSLSCAASGRTFSASAMAWFRQAPGTDREFVAAVAWGGRDT |
| | TP7-75B | HYADSVKGRFTISMNNAKNTVVLQMNSLKPEDTAVYYCAAGQRGWSHSTSSDYDYWGQ |
| | | GTQVTVSS |
| 15 | hMSLN- | QVQLVESGGGLVQAGGSLRLSCAASGSIRSASGMHWYRQAPGKERELVASITTSGETT |
| | mTP7-82 | WYRDSVKGRFTISINNAKMMLYLQMNSLKFEDTAVYYCNLRSYWGQGTQVTVSS |

Table 2 provides correspondence between antibody names used herein with V_{H}H #, and SEQ ID NOs for CDR1, CDR2, CDR3, and full-length sequences for each sdAb.

TABLE 2
VHH Sequence ID Numbers

Sequence ID Nos.

| Name | CDR1 | CDR2 | CDR3 | Full Length |
|---|---|---|---|---|
| TP7-4 | 1 | 2 | 3 | 46 |
| TP7-5 | 4 | 5 | 6 | 47 |
| TP7-9 | 7 | 8 | 9 | 48 |
| TP7-18 | 10 | 11 | 12 | 49 |
| TP7-35 | 13 | 14 | 15 | 50 |
| TP7-38A | 16 | 17 | 18 | 51 |
| TP7-38B | 19 | 20 | 21 | 52 |
| TP7-56 | 22 | 23 | 24 | 53 |
| bioTP7-7 | 25 | 26 | 27 | 54 |
| biomTP7-41 | 28 | 29 | 30 | 55 |
| bioTP7-48 | 31 | 32 | 33 | 56 |
| bioTP7-53 | 34 | 35 | 36 | 57 |
| TP7-75A | 37 | 38 | 39 | 58 |
| TP7-75B | 40 | 41 | 42 | 59 |
| mTP7-82 | 43 | 44 | 45 | 60 |
| Consensus | 86 | 87 | 88 | n/a |
| TP7-5&TP7-56 | | | | |

### Recombinant Polypeptides

In one aspect, there is provided a recombinant polypeptide comprising an sdAb as defined herein. In one embodiment, there is provided a recombinant polypeptide comprising one or more sdAb as defined herein. In one embodiment, there is provided a recombinant polypeptide comprising two or more sdAbs as defined herein. In one embodiment, there is provided a recombinant polypeptide comprising more than two sdAbs as defined herein.

### V_{H}H:Fc Fusions

In one aspect, there is provided the sdAb defined herein fused to a human Fc (termed a “V_{H}H:Fc fusion”). For example, the V_{H}H:Fc fusion may comprise at least a CH2 and a CH3 of the IgG, IgA, or IgD isotype. The V_{H}H:Fc fusion may comprise at least a CH2, a CH3, and a CH4 of the IgM or IgE isotype. Such embodiments may be useful in activating the immune system in higher order recombinant molecules. For example, according to some embodiments, two such Fc-containing V_{H}H:Fc fusions may assemble to form a recombinant monomeric antibody. In some embodiment, such a monomeric antibody is capable of activating the immune system. Such monomeric antibodies may be of lgG, IgA, IgD, IgE, or IgM isotype. In one embodiment, IgA Fc-containing V_{H}H:Fc fusions may also assemble into a recombinant dimeric (secretory) form. Multimeric forms are also envisaged in some embodiments. For example, IgM monomer chains may assemble to form a recombinant pentameric antibody.

In some embodiments, the multivalent antibody described herein may be an assembly of the same V_{H}H:Fc fusions.

In some embodiments, the multivalent antibody described herein may be an assembly of the different V_{H}H:Fc fusions having the same binding target. For example, these may bind to different epitopes on the same target molecule. Examples may include assemblies of different V_{H}H:Fc fusions, each comprising a different anti-MSLN sdAb as defined herein.

In some embodiments, the multivalent antibody described herein may be an assembly of an V_{H}H:Fc fusion defined herein (comprising an anti-MSLN sdAb as defined herein) and another V_{H}H:Fc fusion comprising a paratope directed to a different target.

### Fusions to Cargo Molecules

In a further aspect, the present disclosure provides anti-MSLN sdAb as defined herein linked to a cargo molecule. The cargo molecule may comprise, for example, a therapeutic moiety, such as for example, a cytotoxic agent, a cytostatic agent, an anti-cancer agent or a radiotherapeutic. In particular embodiments of the disclosure, the antibody drug conjugates may comprise a cytotoxic agent. Another particular embodiment of the disclosure relates to antibody drug conjugates comprising a radiotherapeutic.

### Recombinant Nucleic Acid Molecules

In one aspect, there is provided a recombinant nucleic acid molecule encoding an sdAb, the recombinant polypeptide, or the V_{H}H:Fc fusion as defined herein.

### Compositions

In one aspect, there is provided a composition comprising the sdAb as defined herein, or a polypeptide comprising such an sdAb; together with an acceptable excipient, diluent or carrier. In one embodiment the composition is a pharmaceutical composition, and the excipient, diluent or carrier is a pharmaceutically acceptable excipient, diluent or carrier.

### Uses and Methods

In one aspect, there is provided a use of the sdAb as defined herein, or of an antibody comprising one or more V_{H}H or V_{H}H:Fc fusion comprising the sdAb as defined herein, for treatment of solid cancer or a hematological malignancy. In one embodiment, the cancer or hematological malignancy comprises cells that express MSLN. In one embodiment, the cancer or hematological malignancy comprises cells that aberrantly over-express MSLN compared to corresponding healthy cells. In one embodiment, the cancer is pancreatic adenocarcinoma, mesothelioma, ovarian cancer or lung cancer. In one embodiment, the malignancy is hematological malignancy, such as, for example acute myeloid leukemia

(AML).

In one aspect, there is provided a use of the sdAb as defined herein, or of an antibody comprising one or more V_{H}H or V_{H}H:Fc fusion comprising the sdAb as defined herein, for preparation of a medicament for treatment of solid cancer or a hematological malignancy. In one embodiment, the cancer or hematological malignancy comprises cells that express MSLN. In one embodiment, the cancer or hematological malignancy comprises cells that aberrantly over-express MSLN compared to corresponding healthy cells. In one embodiment, the cancer is pancreatic adenocarcinomas, mesotheliomas, ovarian cancer or lung cancer. In one embodiment, the malignancy is hematological malignancy including acute myeloid leukemia (AML).

In one aspect, there is provided the sdAb as defined herein, or of an antibody comprising one or more V_{H}H or V_{H}H:Fc fusion comprising the sdAb as defined herein, for use in treatment of solid cancer or a hematological malignancy. In one embodiment, the cancer or hematological malignancy comprises cells that express MSLN. In one embodiment, the cancer or hematological malignancy comprises cells that aberrantly over-express MSLN compared to corresponding healthy cells. In one embodiment, the cancer is pancreatic adenocarcinomas, mesotheliomas, ovarian cancer or lung cancer. In one embodiment, the malignancy is hematological malignancy including acute myeloid leukemia (AML).

In one aspect, there is provided a method of treating solid cancer or a hematological malignancy in subject comprising administering to the subject the sdAb as defined herein, or of an antibody comprising one or more V_{H}H or V_{H}H:Fc fusion comprising the sdAb as defined herein. In one embodiment, the cancer or hematological malignancy comprises cells that express MSLN. In one embodiment, the cancer or hematological malignancy comprises cells that aberrantly over-express MSLN compared to corresponding healthy cells. In one embodiment, the cancer is pancreatic adenocarcinomas, mesotheliomas, ovarian cancer or lung cancer. In one embodiment, the malignancy is hematological malignancy including acute myeloid leukemia (AML).

In one aspect, there is provided a use of the sdAb as defined herein, or of an antibody comprising one or more V_{H}H or V_{H}H:Fc fusion comprising the sdAb as defined herein, for delivery of a lipid nanoparticle (LNP) to a cell. The sdAb, or the antibody comprising the one or more V_{H}H or V_{H}H:Fc fusion may be conjugated to, tethered to, or otherwise attached to the LNP.

In one aspect, there is provided a use of the sdAb as defined herein, or of an antibody comprising one or more V_{H}H or V_{H}H:Fc fusion comprising the sdAb as defined herein, for preparation of a medicament for delivery of a lipid nanoparticle (LNP) to a cell. The sdAb, or the antibody comprising the one or more VAH or V_{H}H:Fc fusion may be conjugated to, tethered to, or otherwise attached to the LNP.

In one aspect, there is provided the sdAb as defined herein, or of an antibody comprising one or more V_{H}H or V_{H}H:Fc fusion comprising the sdAb as defined herein, for use in delivery of a lipid nanoparticle (LNP) to a cell. The sdAb, or the antibody comprising the one or more V_{H}H or V_{H}H:Fc fusion may be conjugated to, tethered to, or otherwise attached to the LNP.

In one aspect, there is provided a method of delivering an LNP to a cell comprising contacting the cell with the sdAb as defined herein, or of an antibody comprising one or more V_{H}H or V_{H}H:Fc fusion comprising the sdAb as defined herein. The sdAb, or the antibody comprising the one or more V_{H}H or V_{H}H:Fc fusion may be conjugated to, tethered to, or otherwise attached to the LNP.

### Multivalent Antibodies and Related Embodiments

In one aspect, there is provided a multivalent antibody comprising an sdAb as defined above.

By “multivalent antibody” is use herein to mean a molecule comprising more than one variable region or paratope for binding to one or more antigen(s) within the same or different target molecule(s).

In some embodiments, the paratopes may bind to different epitopes on the same target molecule. In some embodiments, the paratopes may bind to different target molecules. In these embodiments, the multivalent antibody may be termed bispecific, trispecific, or multispecific, depending on the number of paratopes of different specificity that are present. As the multivalent antibody comprises one of the anti-MSLN sdAbs as herein defined, the multivalent antibody comprises MSLN binding affinity.

For example, as explained above, in some embodiments a multivalent antibody may be an assembly of a V_{H}H or V_{H}H:Fc fusion defined herein (comprising an sdAb as defined herein) and another V_{H}H or V_{H}H:Fc fusion comprising a different paratope conferring a different specificity.

In one embodiment, there is provided a bispecific antibody comprising an sdAb as defined above, and a second antigen-binding portion. In some embodiments, the second antigen binding portion may comprise a monoclonal antibody, an Fab, an F(ab′)₂, an Fab′, an scFv, or a sdAb, such as a V_{H}H or a VNAR.

An “antigen-binding portion” is meant a polypeptide that comprises an antibody or antigen-binding fragment thereof having antigen-binding activity, including engineered antibodies fragments thereof.

In some embodiments, the second antigen-binding portion may bind to human serum albumin, e.g., for the purposes of stabilization/half-life extension.

In one embodiment, there is provided a trispecific antibody comprising an sdAb as defined above, and a second-binding portion, and a third antigen-binding portion. In some embodiments, the second antigen binding portion comprises a monoclonal antibody, an Fab, and F(ab′)₂, and Fab′, an sdFv, or an sdAb, such as a V_{H}H or a V_{NAR}. In some embodiments, the third antigen binding portion comprises, independently, a monoclonal antibody, an Fab, and F(ab′)₂, and Fab′, an sdFv, or an sdAb, such as a V_{H}H or a V_{NAR}.

The second and/or third antigen-binding portion may bind to human serum albumin, e.g., for the purposes of stabilization/half-life extension.

In some embodiments, the trispecific antibody may be multispecific and the antibody may comprise one or more additional antigen-binding portion(s). In such embodiments, the additional antigen-binding portion(s) may be, independently, an Fab, an F(ab′)₂, an Fab′, an sdFv, or an sdAb, such as a V_{H}H or a V_{NAR}.

In one embodiment, the multispecific antibody comprises a first antigen-binding portion comprising an sdAb as defined herein, and a second antigen-binding portion.

In one embodiment, the second antigen-binding moiety binds specifically to a cell-surface marker of an immune cell.

A “cell surface marker” is a molecule expressed at the surface of the cell that is particular to (or enriched in) a cell type, and that is capable of being bound or recognized by an antigen-binding portion.

### Bispecific T-Cell Engager (BITE)

In one embodiment, the multivalent antibody is a bispecific T-cell engager comprising an sdAb as defined herein and second antigen-binding moiety that binds specifically to a cell-surface marker of a T-cell. In one embodiment, the T-cell marker comprises human CD3.

Human CD3, as will be recognized, is a multi-subunit antigen, of which various subunits may participate in CD3 activation. One such subunit is CD3 epsilon (see, e.g., GenBank NP_000724.1). Other non-limiting examples include CD3 gamma (see, e.g., GenBank NP_000064.1) and delta (see, e.g., GenBank NP_000723.1 for delta isoform A, and, e.g., GenBank NP_001035741.1 for delta isoform B).

In some embodiments, T-cell marker comprises CD3 epsilon, CD3 gamma, or CD3 delta. In one specific embodiment, the T-cell marker comprises CD3 epsilon.

The term “bispecific T-cell engager”, as used herein, refers to a recombinant bispecific protein that has two linked variable regions from two different antibodies, one targeting a cell-surface molecule on T cells (for example, CD38), and the other targeting antigens on the surface of disease cells, typically malignant cells. For example, a bispecific T-cell engager may comprise an sdAb as defined herein and a scFvs. A bispecific T-cell engager may comprise an sdAb as defined herein and a second VHH/sdAb. The two variable regions are typically linked together by a short flexible linker such as GlySer linker. This linker may or may not also incorporate a hinge domain derived from various proteins such as human CD8a, human immunoglobulin, or other. By binding to tumor antigens and T cells simultaneously, bispecific T-cell engagers mediate T-cell responses and killing of tumor cells. The T-cell/target cell adherence facilitated by a bispecific T-cell engager is independent of MHC haplotype.

In one embodiment, the bispecific T-cell engager comprises, in N-terminal to C-terminal direction: the first antigen-binding portion, an amino acid linker, and the second antigen-binding portion.

In one embodiment, the bispecific T-cell engager further comprises a signal peptide N-terminal to the first antigen-binding portion.

A “signal peptide”, as referred to herein allows the nascent protein to be directed to the endoplasmic reticulum and subsequently to the cell surface, where it is expressed. The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases. The signal peptide may be at the amino terminus of the molecule.

In one embodiment, the signal peptide is a signal peptide from human CD28. In one embodiment, the signal peptide from human CD28 comprises SEQ ID NO: 680. In one embodiment, the signal peptide is at least 80% identical, at least 90% identical, at least 95% identical, or at least 98% identical to SEQ ID NO: 68.

By “amino acid linker”, in this context, will be understood a sequence of sufficient length, flexibility, and composition to permit the bispecific T-cell engager to be properly functional an engage with both targets.

The amino acid linker may comprise a hinge. The hinge may be from human CD8, e.g. as set forth in SEQ ID NO: 70.

In one embodiment, the multivalent antibody is encoded by SEQ ID NO: 81.

An example is provided as SEQ ID NO: 82, wherein the sequence encoding an sdAb as described herein (e.g., any one of SEQ ID NO: 46 to 60) is followed by a sequence encoding a linker (SEQ ID NO: 69 or 70) followed by a sequence encoding CD3-scFv (SEQ ID NO: 81).

Another example is provided in SEQ ID NO: 83, wherein a dimeric T-cell engager is created by linking a MSLN-specific sdAb as described herein (e.g., any one of SEQ ID NO: 46 to 60) followed by a sequence encoding a linker (SEQ ID NO: 69 or 70) followed by a sequence encoding CD3-scFv (SEQ ID NO: 81), followed by a linker (SEQ ID NO: 69 or 70), followed by a human-Fc domain (SEQ ID NO: 84)

In some embodiments, the bi-specific T-cell engager is a sequence variant of the above bi-specific T-cell engager having 80%, 90%, 95%, 98%, or 99% identity to one of the above-described bi-specific T-cell engagers. In some embodiments, the variant retains substantially the same binding specificity as the parent molecule from which it is derived.

### BiKEs & TriKEs

In one embodiment, the multivalent antibody is a bispecific killer cell engager.

The term “BIKE” refers to a recombinant bispecific protein that has two linked variable regions from two different antibodies, one targeting a cell-surface molecule on natural killer (NK) cells (for example, CD16), and the other targeting antigens on the surface of disease cells, typically malignant cells. For example, the BiKE may comprise two scFvs, two VHHs, or a combination thereof. The two are typically linked together by a short flexible linker. By binding to tumor antigens and NK cells simultaneously, BiKEs mediate NK-cell responses and killing of tumor cells.

In one embodiment, the cell-surface marker of the immune cell comprises a natural killer (NK) cell marker. In one embodiment, the NK cell marker comprises human CD16.

In one embodiment, the multivalent antibody is a trispecific killer cell engager (TriKE).

The term “TriKE” indicates at a BIKE that has been further modified to include another functionality. This term has been used to encompass various approaches. One approach involves inserting an intervening immunomodulatory molecule (a modified human IL-15 crosslinker) to promote NK cell activation, expansion, and/or survival (Vallera et al. IL-15 Trispecific Killer Engagers (TrikEs) Make Natural Killer Cells Specific to CD33+ Targets While Also Inducing In Vivo Expansion, and Enhanced Function. Clinical Cancer Research. 2012; 22 (14): 3440-50). Other TriKE approaches are trispecific molecules that include three antibody variable regions: one targeting an NK cell receptor and two that target tumour-associated antigens (Gleason et al. Bispecific and Trispecific Killer Cell Engagers Directly Activate Human NK Cells through CD16 Signaling and Induce Cytotoxicity and Cytokine Production. Mol Cancer Ther. 2012; 11 (12): 2674-84). Yet other TriKE approaches target two NK cell receptors (e.g., CD16 and NKp46) and one tumour-associated antigen (Gauthier et al. Multifunctional Natural Killer Cell Engagers Targeting NKp46 Trigger Protective Tumor Immunity. Cell. 2019; 177 (7): 1701-13).

An example is provided as SEQ ID NO: 85, wherein the sequence encoding an sdAb as described herein (e.g., any one of SEQ ID NO: 46 to 60) is followed by a sequence encoding a linker (SEQ ID NO: 69 or 70) followed by a sequence encoding any NK-targeting antibody fragment (e.g. NKp30).

In one embodiment, the multivalent antibody further comprises a cytokine for stimulating activation, expansion, and/or survival of NK cells. In one embodiment, the cytokine for stimulating expansion of NK cells is interleukin-15 (IL15), a variant thereof, or a functional fragment thereof.

In one embodiment, the multivalent antibody further comprises at least a third antigen-binding portion that binds to a second NK cell marker. In one embodiment, the second NK cell marker is human NKp46.

In one embodiment, the multivalent antibody further comprises at least a third antigen-binding portion that binds to a tumour-associated antigen. In some embodiment, the tumour-associated antigen is distinct from human MSLN.

In one embodiment, the third antigen-binding portion comprises a V_{H}H, a V_{NAR}, or an scFv.

In one embodiment, the second antigen-binding portion comprises a V_{H}H.

In one embodiment, the third antigen-binding portion binds to human serum albumin. In such embodiment, the affinity for human serum albumin may contribute to stabilization/increased half-life.

In one embodiment, the multivalent antibody comprises any one of SEQ ID NO: 77 to 80 and 82; or a sequence at least 80% identical thereto. In one embodiment, the multivalent antibody comprises any one of SEQ ID NO: 77 to 80 and 82; or a sequence at least 90% identical thereto. In one embodiment, the multivalent antibody comprises any one of SEQ ID NO: 77 to 80 and 82; or a sequence at least 95% identical thereto. In one embodiment, the multivalent antibody comprises any one of SEQ ID NO: 77 to 80 and 82; or a sequence at least 98% identical thereto. In one embodiment, the multivalent antibody comprises any one of SEQ ID NO: 77 to 80 and 82.

In some embodiments, the BiKE or TriKE is a sequence variant of one of the above BiKEs and TriKEs having 80%, 90%, 95%, 98%, or 99% identity thereto. In some embodiments, the variant retains substantially the same binding specificity as the parent molecule from which it is derived. In some embodiments the variant retains substantially the same binding affinity as the parent molecule from which it is derived.

### Recombinant Nucleic Acid Molecules

In aspect, there is provided a recombinant nucleic acid molecule encoding the multivalent antibody as defined herein. In one embodiment, nucleic acid is a vector.

### Compositions

In one aspect, there is provided a composition comprising a multivalent antibody as defined herein; together with an acceptable excipient, diluent or carrier. In one embodiment, the composition comprises a bispecific T-cell engager as herein defined. In one embodiment, the composition comprises a BIKE as herein defined. In one embodiment, the composition comprises a TriKE as herein defined. In one embodiment the composition is a pharmaceutical composition, and the excipient, diluent or carrier is a pharmaceutically acceptable excipient, diluent or carrier.

### Uses and Methods

In one aspect, there is provided a use of the multivalent antibody as defined herein for treatment of solid cancer or a hematological malignancy. In one embodiment, the cancer is pancreatic adenocarcinomas, mesotheliomas, ovarian cancer or lung cancer. In one embodiment, the malignancy is hematological malignancy including acute myeloid leukemia (AML).

In one aspect, there is provided a use of the multivalent antibody as defined herein for preparation of a medicament for treatment of a cancer or a hematological malignancy.

In one aspect, there is provided the multivalent antibody as defined herein for use in treatment of a cancer or a hematological malignancy. In one embodiment, the cancer is pancreatic adenocarcinomas, mesotheliomas, ovarian cancer or lung cancer. In one embodiment, the malignancy is hematological malignancy including acute myeloid leukemia (AML).

In one aspect, there is provided a method of treating a cancer or a hematological malignancy in subject comprising administering to the subject the multivalent antibody as defined herein. In one embodiment, the cancer is pancreatic adenocarcinomas, mesotheliomas, ovarian cancer or lung cancer. In one embodiment, the malignancy is hematological malignancy including acute myeloid leukemia (AML).

In some embodiments of the multivalent antibodies, recombinant nucleic acids, compositions, uses, and methods disclosed herein, the sdAb in the multivalent antibody comprises a CDR1 amino acid sequence as set forth in SEQ ID NO: 22, a CDR2 amino acid sequence as set forth in SEQ ID NO: 23, and a CDR3 amino acid sequence as set forth in SEQ ID NO: 24 (hMSLN-bioTP7-7) In some embodiments of the above uses and methods, the sdAb in the multivalent antibody comprises SEQ ID NO: 54 (hMSLN-bioTP7-7).

### Chimeric Antibody Receptors & Related Embodiments

In one aspect, there is provided a chimeric antibody receptor (CAR), which binds to human MSLN, comprising the VHH sdAb as defined herein.

“Chimeric antigen receptors” are receptor proteins engineered to give immune cells the new ability to target a specific protein. The receptors are chimeric because they combine both antigen-binding and immune-cell activating functions into a single receptor (see Stoiber et al. Limitations in the Design of Chimeric Antigen Receptors for Cancer Therapy. Cells. 2012; 8 (5): 472 and van der Stegen et al. The pharmacology of second-generation chimeric antigen receptors. Nat Rev Drug Discov. 2019; 14 (7): 499-509).

In one embodiment, the CAR comprises, in N-terminal to C-terminal directiona MSLN binding domain comprising: the sdAb as defined herein, a polypeptide hinge, a transmembrane domain, and a cytoplasmic domain comprising a co-stimulatory domain and a signaling domain.

The term “polypeptide hinge” used herein generally means any oligo- or polypeptide that functions to link the extracellular ligand-binding domain to the transmembrane domain. In particular, hinge regions are used to provide more flexibility and accessibility for the extracellular ligand-binding domain. A hinge region may comprise up to 300 amino acids, preferably 10 to 100 amino acids and most preferably 25 to 50 amino acids. Hinge region may be derived from all or part of naturally occurring molecules, such as from all or part of the extracellular region of CD8, CD4 or CD28, or from all or part of an antibody constant region. Alternatively, the hinge region may be a synthetic sequence that corresponds to a naturally occurring hinge sequence, or may be an entirely synthetic hinge sequence.

In one embodiment, the polypeptide hinge is a CD8 hinge domain. In one embodiment, the CD8 hinge domain comprises SEQ ID NO: 70.

The term “transmembrane domain” indicates a polypeptide having the ability to span a cell membrane and thereby link the extracellular portion of the CAR (which comprises the MSLN-binding portion) to the intracellular portion responsible for signaling. Commonly used transmembrane domains for CARs have been derived from CD4, CD8a, CD28 and CD3Z.

In one embodiment, the transmembrane domain is a CD28 transmembrane domain. In one embodiment, the CD28 transmembrane domain comprises SEQ ID NO: 72.

In one embodiment, the transmembrane domain is at least 80%, at least 90%, at least 95%, or at least 98% identical to SEQ ID NO: 72.

In one embodiment, the transmembrane domain is a CD8 transmembrane domain. In one embodiment, the CD8 transmembrane domain comprises SEQ ID NO: 71. In one embodiment, the transmembrane domain is at least 80%, at least 90%, at least 95%, or at least 98% identical to SEQ ID NO: 71.

The term “cytoplasmic domain” (also termed a “signal transduction domain”) refers to the intracellular portion of the CAR that is responsible for intracellular signaling following the binding of extracellular ligand binding domain to the target resulting in the activation of the immune cell and immune response. In other words, cytoplasmic domain is responsible for the activation of at least one of the normal effector functions of the immune cell in which the CAR is expressed. For example, the effector function of a T cell can be a cytolytic activity or helper activity including the secretion of cytokines. Thus, the term “cytoplasmic domain” refers to the portion of a protein which transduces the effector signal and directs the cell to perform a specialized function. It is common for such cytoplasmic domains to comprise a co-stimulatory domain in addition to a signaling domain.

The term “signaling domain” refers to the portion of a protein which transduces the effector signal and directs the cell to perform a specialized function. Examples of signal transducing domain for use in a CAR can be the cytoplasmic sequences of the T cell receptor and co-receptors that act in concert to initiate signal transduction following antigen receptor engagement, as well as any derivate or variant of these sequences and any synthetic sequence that has the same functional capability. Signal transducing domain comprises two distinct classes of cytoplasmic signaling sequence, those that initiate antigen-dependent primary activation, and those that act in an antigen-independent manner to provide a secondary or co-stimulatory signal. Primary cytoplasmic signaling sequence can comprise signaling motifs which are known as immunoreceptor tyrosine-based activation motifs or ITAMs. ITAMs are well defined signaling motifs found in the intracytoplasmic tail of a variety of receptors that serve as binding sites for syk/zap70 class tyrosine kinases. Non-limiting examples of signaling domains used in the invention can include those derived from TCRzeta, common FcR gamma (FCERIG), Fcgamma RIIa, FcRbeta (Fc Epsilon Rib), FcRepsilon, CD3 zeta, CD3gamma, CD3delta, CD3epsilon, CD5, CD22, CD79a, CD79b, CD66d, DAP10, or DAP12. In a preferred embodiment, the signaling transducing domain of the CAR can comprise the CD3zeta signaling domain.

In one embodiment, the signaling domain is a CD3-zeta signaling domain. In one embodiment, the CD3-zeta signaling domain comprises SEQ ID NO: 74. In one embodiment, the signaling domain is at least 80%, at least 85%, at least 90%, at least 95%, or at least 98% identical to SEQ ID NO: 74.

The term “co-stimulatory domain” refers to the cognate binding partner on a T-cell that specifically binds with a co-stimulatory ligand, thereby mediating a co-stimulatory response by the cell, such as, but not limited to proliferation. Co-stimulatory molecules include, but are not limited to, an MHC class I molecule, BTLA and Toll ligand receptor. Examples of costimulatory molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3 and a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CDIId, ITGAE, CD103, ITGAL, CDIIa, LFA-1, ITGAM, CDIIb, ITGAX, CDIIc, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D or a combination thereof.

In one embodiment, the co-stimulatory domain is a 4-1BB co-stimulatory domain. In one embodiment, the 4-1BB signal transduction domain comprises SEQ ID NO: 73. In one embodiment, the co-stimulatory domain is at least 80%, at least 85%, at least 90%, at least 95%, or at least 98% identical to SEQ ID NO: 73.

In one embodiment, CAR further comprises a flexible amino acid linker between the sdAb and the polypeptide hinge. In one embodiment, the amino acid linker comprises SEQ ID NO: 69. In one embodiment, the amino acid linker is at least 80%, at least 85%, at least 90%, at least 95%, or at least 98% identical to SEQ ID NO: 69.

In one embodiment, the CAR further comprises a signal peptide.

In one embodiment, the signal peptide is a signal peptide from human CD28. In one embodiment, the signal peptide from human CD28 comprises SEQ ID NO: 60. In one embodiment, the signal peptide is at least 80%, at least 85%, at least 90%, at least 95%, or at least 98% identical to SEQ ID NO: 68.

In one embodiment, the CAR is encoded by SEQ ID NO: 67, which comprises a sequence encoding any one of the sdAbs as described herein.

In one embodiment, the CAR comprises any one of SEQ ID NOs: 61 to 66; or a sequence at least 80%, at least 85%, at least 90%, at least 95%, or at least 98% identical to SEQ ID NOs: 61 to 66.

In one embodiment, the CAR is encoded by SEQ ID NO: 67, which comprises a sequence encoding any one of the sdAbs described herein.

In one embodiment, the CAR further comprises a second MSLN binding domain positioned N-terminally or C-terminally with respect to the first MSLN binding domain, and may be spaced apart from the first MSLN binding domain by an amino acid linker.

In one embodiment, the second MSLN binding domain comprises and sdAb that is the same as the sdAb of the first MSLN binding domain. These embodiments are referred to herein as “double binders”.

In another embodiment, the second MSLN binding domain comprises an sdAb that is different to the sdAb of the first MSLN binding domain. These embodiments are referred to herein as “bi-paratopic”. In this embodiment, the sdAb of the second MSLN binding domain may bind to a different epitope of MSLN to that bound by the sdAb of the first MSLN binding domain. A “different epitope” may alternatively be an epitope that overlaps that bound by the sdAb of the first MSLN binding domain. Alternatively, the sdAb may bind to the same epitope to that bound by the sdAb of the first MSLN binding domain.

In one embodiment, the CAR further comprises an additional binding domain that binds to a target molecule other than MSLN. These embodiments are referred to herein as “tandem constructs”. The additional binding domain may comprise an additional sdAb or a scFv. The additional binding domain may be positioned N-terminally or C-terminally with respect to the MSLN binding domain. The additional binding domain may be separated from the MSLN binding domain by an amino acid linker. In one embodiment, the target molecule bound by the additional binding domain is expressed by a target cell that also expresses MSLN, thereby providing a CAR having dual affinity for the same target cell. For example, the target molecule other than MSLN may be EGFR, Mucin-1, HER2, EGFRvIII, FAP, or CD3.

In some embodiments, the tandem constructs may comprise a third binding domain that targets yet another target molecule distinct from MSLN and distinct from that bound by additional binding domain. Such constructs are referred to herein as “multi-binders”.

In some embodiments, the CAR is a sequence variant of one of the above CARs having 80%, 90%, 95%, 98%, or 99% identity thereto. In some embodiments, the variant retains substantially the same binding specificity as the parent molecule from which it is derived. In some embodiments the variant retains substantially the same binding affinity as the parent molecule from which it is derived.

### Nucleic Acids and Vectors

In one aspect, there is provided a recombinant nucleic acid molecule (either DNA or RNA) encoding the CAR as defined herein.

In one aspect, there is provided a vector comprising the recombinant nucleic acid molecule as defined herein. In one embodiment, the vector is a viral vector. In one embodiment, the viral vector is a lentivirus vector.

### Viral Particles

In one aspect, there is provided a recombinant viral particle comprising the recombinant nucleic acid as defined herein. In one embodiment, the recombinant viral particle is a recombinant lentiviral particle.

### Cells

In one aspect, there is provided a cell comprising the recombinant nucleic acid molecule as defined herein.

In one aspect, there is provided an engineered cell expressing at the cell surface membrane the CAR as defined herein. In one embodiment, the engineered cell is an immune cell. In one embodiment, the immune cell is a T-lymphocyte or is derived from T-lymphocytes.

### Use and Methods

“CAR-T” cell therapy uses T cells engineered with CARs for cancer therapy. The premise of CAR-T immunotherapy is to modify T cells to recognize disease cells, typically cancer cells, in order to more effectively target and destroy them. Generally, T cells are genetically altered to express a CAR, and these cells are infused into a patient to attack their tumors. CAR-T cells can be either derived from T cells in a patient's own blood (autologous) or derived from the T cells of another healthy donor (allogeneic).

“CAR-NK” cell therapy uses natural killer (NK) cells engineered to express CAR constructs for cancer therapy. The premise of CAR-NK immunotherapy is to modify NK cells to recognize disease cells, typically cancer cells, in order to more effectively target and destroy them.

CAR-related applications involving different cells types are also possible, such as, for example, macrophages (CAR-macrophage or CAR-M), or neutrophils (CAR-neutrophil). CAR-related applications involving stem cells are also possible, such as, induced pluripotent stem cell (CAR-iPSC), which could be differentiated into mature leukocytes in vitro. Collectively, these are referred to as “CAR applications”. It is to be appreciated that generation of suitable cells may involve collection and engineering of appropriate precursors, such as monocytes in the case of CAR-M applications.

Generally, the cells targeted by these approaches will express MSLN. In some embodiments, the cells aberrantly over-express MSLN compared to corresponding healthy cells. In some embodiments, the cancer is a hematological malignancy. In some embodiments, the hematological malignancy is AML. In some embodiments, the cancer is pancreatic cancer, lung cancer, or ovarian cancer.

In one aspect, there is providing a use of the nucleic acid, vector, or viral particle as described herein for preparation of cells for a CAR application. In one embodiment, the CAR application is CAR-T. In one embodiment, the CAR application is CAR-macrophage. In one embodiment, the CAR application is CAR-NK. In one embodiment, the CAR application is CAR-neutrophil. In one embodiment, the CAR application is CAR-iPSC.

In one aspect, there is providing a method of preparing cells for a CAR application comprising contacting a cell with the viral particle as described herein. In one embodiment, the cell is a leukocyte. In one embodiment, the leukocyte is a T-cell. In one embodiment, the leukocyte is a monocyte. In one embodiment, the leukocyte is a macrophage. In one embodiment, the leukocyte is an NK cell. In one embodiment, the leukocyte is a neutrophil. In one embodiment, the cell is an iPSC. In one embodiment, the cell is from a donor. In one embodiment, the cell is from a patient.

In one aspect, there is providing a method of preparing cells for a CAR application comprising introducing into a cell the nucleic acid or vector as described herein. In one embodiment, the cell is a leukocyte. In one embodiment, the leukocyte is a T-cell. In one embodiment, the leukocyte is a monocyte. In one embodiment, the leukocyte is a macrophage. In one embodiment, the leukocyte is an NK cell. In one embodiment, the leukocyte is a neutrophil. In one embodiment, the cell is an iPSC. In one embodiment, the cell is from a donor. In one embodiment, the cell is from a patient.

In one aspect, there is provided a use of the CAR or of the engineered cell as described herein for treatment of a solid cancer. In one embodiment, the cancer is a hematological malignancy. In one embodiment, the cancer is pancreatic adenocarcinomas, mesotheliomas, ovarian cancer or lung cancer. In one embodiment, the hematological malignancy is acute myeloid leukemia (AML). In one embodiment, the cell is a leukocyte. In one embodiment, the leukocyte is a T-cell. In one embodiment, the leukocyte is a monocyte. In one embodiment, the leukocyte is a macrophage. In one embodiment, the leukocyte is an NK cell. In one embodiment, the leukocyte is a neutrophil. In one embodiment, the cell is an iPSC.

In one embodiment, the method further comprises an initial step of obtaining cells from a patient or donor and introducing the recombinant nucleic acid molecule or vector encoding the CAR, as described herein.

In one embodiment, the method further comprises an initial step of obtaining cells from a patient or donor and contacting the cells with the viral particle, as described herein.

In one aspect, there is provided a use of the CAR or of the engineered cell as described herein for preparation of a medicament treatment of a solid cancer. In one embodiment, the cancer is a hematological malignancy. In one embodiment, the cancer is pancreatic adenocarcinomas, mesotheliomas, ovarian cancer or lung cancer. In one embodiment, the hematological malignancy is acute myeloid leukemia (AML). In one embodiment, the cell is a leukocyte. In one embodiment, the leukocyte is a T-cell. In one embodiment, the leukocyte is a monocyte. In one embodiment, the leukocyte is a macrophage. In one embodiment, the leukocyte is an NK cell. In one embodiment, the leukocyte is a neutrophil. In one embodiment, the cell is an iPSC.

In one aspect, there is provided the CAR or the engineered cell as described herein for use in treatment of a solid cancer. In one embodiment, the cancer is a hematological malignancy. In one embodiment, the cancer is pancreatic adenocarcinomas, mesotheliomas, ovarian cancer or lung cancer. In one embodiment, the hematological malignancy is acute myeloid leukemia (AML). In one embodiment, the cell is a leukocyte. In one embodiment, the leukocyte is a T-cell. In one embodiment, the leukocyte is a monocyte. In one embodiment, the leukocyte is a macrophage. In one embodiment, the leukocyte is an NK cell. In one embodiment, the leukocyte is a neutrophil. In one embodiment, the cell is an iPSC.

In one aspect there is provided a method of treating a solid cancer in a subject, comprising administering to the subject the engineered cell as defined herein. In one embodiment, the hematological malignancy is acute myeloid leukemia (AML). In one embodiment, the cell is a leukocyte. In one embodiment, the leukocyte is a T-cell. In one embodiment, the leukocyte is a monocyte. In one embodiment, the leukocyte is a macrophage. In one embodiment, the leukocyte is an NK cell. In one embodiment, the leukocyte is a neutrophil. In one embodiment, the cell is an iPSC.

In certain embodiments of the CAR constructs, recombinant nucleic acids, vectors, viral particles, cells, uses, and methods described herein, the sdAb in the CAR comprises a CDR1 amino acid sequence as set forth in SEQ ID NO: 19, a CDR2 amino acid sequence as set forth in SEQ ID NO: 20, and a CDR3 amino acid sequence as set forth in SEQ ID NO: 21 (hMSLN-TP7-56). In certain embodiments of the above CAR constructs, uses, and methods, the sdAb in the CAR comprises SEQ ID: 40 (hMSLN-TP7-56).

### EXAMPLES

The following Examples outline embodiments of the invention and/or studies conducted pertaining to the invention. While the Examples are illustrative, the invention is in no way limited the following exemplified embodiments.

Mesothelin (MSLN) is a differentiation antigen present at low or limited levels on a restricted set of normal adult tissues but aberrantly overexpressed in 80-90% of pancreatic adenocarcinomas and mesotheliomas, in 60-65% of ovarian cancers, in 60-70% of lung cancers and in hematological malignancies such as in acute myeloid leukemia (AML). MSLN is also shown to be involved in tumor cell proliferation and resistance to chemotherapy. Therefore, the limited distribution of mesothelin on normal tissues and its overexpression in a wide range of tumor types makes it a promising target for tumor-specific therapy.

MSLN has emerged as a molecular target of interest, in particular, for solid tumors. More than a dozen clinical trials are ongoing or completed to study various mesothelin targeting strategies in the context of solid tumours or AML, though none have progressed to regulatory approval in the United States as of August 2022.

The applicability of camelid single domain antibodies as soluble, stable and modular domains for a number of therapeutic applications has well-been established with the first FDA-approved bivalent nanobody in 2018. Therefore, nanobodies present an excellent building block in CAR-T molecules, allowing a simple antibody domain fusion and building a pool of more stable and functional CAR-T constructs, therefore, increasing the chance of screening much more effective CAR-T cells for the treatment of non-solid tumor cells.

In addition, these nanobodies could also be utilized to develop additional safe and efficacious immunotherapy regimens including but not limited to naked or drug conjugated antibody therapies and specific immune cell engager therapeutics.

The approaches described herein use single domain antibodies (sdAb) derived from an immunized llama with a unique MSLN-ECD domain expressed in CHO cells at NRC-HHT. These sdAb sequences specifically bind to MSLN antigen with high affinities which is over-expressed in many types of solid tumors and hematological malignancies in humans. Using the sdAb sequences, a novel chimeric receptor sequence has been generated that combines MSLN specific sdAb with T cell signaling molecules (in the form of 41BB, CD28 or other co-stimulation domain and CD3zeta signaling domains). In addition to chimeric antigen receptor applications, these MSLN targeting antibodies may be useful for developing other forms of immunotherapies including but not limited to bi-specific/tri-specific T or NK cell engager applications, antibody-drug conjugates, or as naked antibodies.

### Example 1: sdAb Antibody Production

### Introduction

Mesothelin (MSLN) is a differentiation antigen present at low or limited levels on a restricted set of normal adult tissues but aberrantly overexpressed in 80-90% of pancreatic adenocarcinomas and mesotheliomas, in 60-65% of ovarian cancers, in 60-70% of lung cancers and in hematological malignancies such as in acute myeloid leukemia (AML). MSLN is also shown to be involved in tumor cell proliferation and resistance to chemotherapy. Therefore, the limited distribution of mesothelin on normal tissues and its overexpression in a wide range of tumor types makes it a promising target for tumor-specific therapy. In fact, MSLN has been selected as a reliable target for cancer immunotherapy and a number of anti-MSLN therapeutic approaches have been tested in human clinical trials.

These include monoclonal antibodies, antibody-drug conjugates, immunotoxins, vaccines and CAR-T cell therapies. Despite all these efforts, no mesothelin targeted therapy has yet been approved for use in clinic. Generating novel antibodies targeting mesothelin; especially single domain antibodies which have competitive advantage over conventional monoclonal antibodies (mAbs) and fragment thereof and are more amenable to generating multi-paratopic/multispecific constructs would be of extreme value. Additionally, these antibody domains could have other potential application such as building blocks in CART-cells, bi-specific immune cell engagers, antibody drug conjugate (ADC) or for targeting nanoparticles loaded with therapeutic cargo to cancer cells expressing MSLN.

Single domain antibodies (sdAbs) (also known as VHHs or nanobodies) derived from the variable domains of the camelid heavy chain, are characteristically stable and fully capable of antigen binding in the absence of the former VL domain.

Applicant has generated functional camelid sdAbs against the ecto-domain of MSLN that present at low or limited levels on a restricted set of normal adult tissues but aberrantly overexpressed in many types of solid tumors including pancreatic adenocarcinomas and mesotheliomas, ovarian and lung cancers, and in hematological malignancies such as in acute myeloid leukemia (AML). The sdAbs will then be used to develop immunotherapeutics, including, but not limited to, CAR-T therapies, bi-, tri- and multi-specific immune engager therapies, and naked or drug/tracer linked therapeutic antibodies with appropriate human IgG fusions. The sdAb may also be used to target other therapeutic modalities to treat cancer cells. These therapies are intended for use as treatment modalities for cancer, auto-immune and inflammatory diseases. Examples are presented of the use of these sdAb sequences for developing CAR-T and bi-specific immune engagers with effective anti-tumor activity.

### Materials and Methods

### Cloning and Expression of MSLN-ECD

The gene encoding the extracellular domain of human predominant MSLN isoform 1 mature protein (aa 295-606-10×His); Uniprot No: Q13421-1) was cloned into pTT5 NRC proprietary mammalian expression vector. Upon transfection of CHO cells, the cells were grown in a 250 mL flask and the expressed proteins in supernatant were purified by Immunoaffinity chromatography (IMAC), analyzed on SDS-PAGE and characterized by mass spectrometry.

### Llama Immunizations

A llama (LPAR1) was immunized with the hMSLN-ECD (Protein Production Team, HHT-Montreal) and subsequently boosted four times with the same immunogen. For each injection, 100 μg of recombinant MSLN-ECD, in a total volume of 0.5 mL was mixed with an equal volume of complete (first injection) and incomplete Freund's adjuvant (subsequent injections) and was injected, subcutaneously. Five injections were performed at approximately 1-2 week intervals and blood was collected after the third injection and 7 days after the fourth and last injection. The immune response analyzed after fourth and fifth injection showed that a stronger response after fourth injection and, therefore, the PBMC from fourth injection was used for the library construction.

### RNA Isolation and PCR Amplification

Total RNA was isolated from approximately 2×10⁷ lymphocytes collected from day 35 of the immunization protocol with a QIAamp RNA blood mini kit (QIAGEN Sciences, Mississauga, ON) and according to the kit instructions. About 5 μg of total RNA was used as template for first strand cDNA synthesis with an oligo dT primer using a first-strand cDNA synthesis kit (Amersham Biosciences, USA). Based on the Camelidae and llama immunoglobulin databases, three variable domain sense primers (MJ1-3) and two CH2 domain antisense primers (CH2 and CH2b3) were designed (Baral T N et al 2013). The first PCR was performed with the cDNA as template and the variable regions of both conventional (IgG1) and heavy chain antibodies (IgG2 and IgG3) were amplified with combinations of MJ1-3/CH2 and MJ1-3/CH2b primers in two separate reactions. The PCR reaction mixtures contained the following components: 2 μL cDNA, 5 μmol of MJ1-3 primer mixture, 5 μmol of either CH2 or CH2b primer, 5 μL of 10× reaction buffer, 3 μL of 2.5 mM dNTP, 2.5 units of Taq DNA polymerase (Roche Applied Science, Indianapolis, IN) and water to a final volume of 50 μL. The PCR protocol consisted of an initial step at 94° C. for 3 minutes followed by 30 cycles of 94° C. for 30 seconds, 55° C. for 30 seconds, 72° C. for 1 minute and a final extension step at 72° C. for 7 minutes. The amplified PCR products were run onto a 2% agarose gel and consisted of two major bands of about 850 bp corresponding to conventional IgG1 and about 600 bp (550-650 bp) corresponding to heavy chain antibodies. The smaller bands were cut out of the gel, purified with a QIAquick gel extraction kit (QIAGEN Inc) and re-amplified in a second PCR reaction containing 1 μL of the purified DNA template, 5 μmol each of MJ7, a VH sense primer with a Sfil restriction site, underlined, (5′-CAT GTG TAG ACT CGC GGC CCA GCC GGC CAT GGC C-3′) and MJ8, an antisense primer with a Sfil restriction enzyme site, underlined, (5′-CAT GTG TAG ATT CCT GGC CGG CCT GGC CTG AGG AGA CGG TGA CCT GG), 5 UL of 10× reaction buffer, 3 L of 2.5 mM dNTP, 2.5 unit of Taq DNA polymerase (Roche Applied Science, Indianapolis, IN) and water to a final volume of 50 μL. The PCR protocol consisted of an initial step at 94° C. for 3 minutes followed by 30 cycles of 94° C. for 30 seconds, 57° C. for 30 seconds, 72° C. for 1 minute and a final extension step at 72° C. for 7 minutes. The amplified PCR products (about 400-450 bp) that correspond to VHH fragments of heavy chain antibodies were purified with a QIAquick PCR purification kit (QIAGEN Inc.), digested with Sfil (New England BioLabs) and re-purified with the same kit.

### Library Construction

Thirty μg of pMED1 (Arbabi-Ghahroudi et al. 2009) DNA was digested with Sfil overnight at 50° C. To minimize the chance of self-ligation, the digestion was continued for additional 2 hours at 37° C. by adding 20 units of both XhoI and PstI restriction enzymes. For library construction, 10 μg of phagemid DNA was ligated with 1.75 μg of V_{H}H fragments and incubated for 2 hours at room temperature using the LigaFast DNA ligation system (Promega, Corp., Madison, WI) and according to the recommended protocol. The ligated product was electroporated into competent E. coli TG1cells (Stratagene, Cedar Creek, TX). Transformed bacterial cells were diluted in SOC medium and incubated for 1 hour at 37° C. with slow shaking. The size of library was calculated by plating aliquots on LB-Amp. The VHH fragments from 96 colonies were PCR-amplified and sequenced for diversity analysis. The library was aliquoted and stored at −80° C.

### Library Panning and Screening

Two Strategies of Biotinylated and Passive Absorption were Used for the Panning of the MSLN-Immune Library:

### a) Passive Panning of MSLN

The constructed LPAR1 Library 2 with a size of 3.3×10⁷ was phage-recued and the phage titer of 1.4×10⁹ cfu/uL. Four rounds of panning was implemented with alternating blocking buffers [e.g. Starter Block (Thermo Fisher Cat #37559) for rounds 1, 3 and 4% milk PBS for rounds 2, 4. The amount of hMSLN passively adsorbed onto Nunc wells decreased from 40 ug to 10 ug over the four rounds of panning (Round1: 40 μg, Round 2: 30 ug; Round 3: 20 μg; and Round 4: 10 μg). A well was first blocked with appropriate buffer for 2 hours at room temperature followed by addition of input phage (~3×10¹¹). In subsequent rounds, ~3×10¹¹ of input phage from each round of amplified phage was used. The incubation was followed by wash steps {R1: 5×PBS-T; 2×PBS: R2: 5×PBS-T; 5×PBS; R3: 7×PBS-T; 5×PBS; and R4: 10× PBS-T; 10× PBS}, and elution with 100 UL of 100 mM TEA. Phage were then removed from wells and neutralized with 50 μL of 1M Tris-HCl PH 7.4 in a new tube. 2 mL of exponentially growing TG1 E. coli culture previously grown at 37° C., 250 rpm, until OD₆₀₀=0.5 in 2YT+2% glucose in a 15 mL Falcon tube, were infected with the eluted phage. A 100 μL aliquot of uninfected TG1 E. coli cells was set aside as a control. Eluted phage were Incubated at 37° C. for 30 minutes with no shaking and then an aliquot was used for titer (dilutions of 102 to 108) and plating on 2YT plates overnight at 32° C. Proceeded with overnight phage amplification using M13KO7 helper phage (~1×10¹⁰ cfu). The next day the eluted titers were calculated to determine the amount of input phage for the subsequent round. The cell culture containing the amplified phage was centrifuged at 5000 rpm, 30 minutes and the supernatant was filtered through 0.22 μM filter unit (Millipore) and precipitated in 20% PEG/2.5M NaCl followed by centrifugation and re-solubilization in PBS (pH7.5). Amplified phage titer was determined (dilutions of 10⁴ to 10¹²) in TG1 E. coli cells as grown previously. After 4 rounds of panning, the sequences of positive colonies from phage ELISA were analyze.

### b) Biotinylated MSLN Panning:

The constructed LPAR1 Library 2 with a size of 3.3×10⁷ was phage-rescued and the phage titer of 1.0×10⁹ cfu/uL was used to pan against the MSLN antigen previously biotinylated by using EZ-link sulfo-NHS-LC-LC-Biotin (Thermoscientific cat #21338). Four rounds of panning were performed with alternating blocking buffers [e.g. Starter Block (Thermo Fisher Cat #37559)] for rounds1, 3 and biotin-free casein for rounds 2, 4. Panning was alternated between both Pierce™ streptavidin coated wells (Round 1, 3) (Thermoscientific cat #15501; lot #TF252884) and Pierce™ neutravidin coated wells (Round 2, 4) (Thermoscientific cat #15508; lot #SK253835). Two wells and either one or two microfuge tubes were first incubated in appropriate blocking buffer overnight at 4° C.

Next day Library input phage (~3×10¹¹) was added to one of the blocked streptavidin wells in appropriate blocking buffer for 1 hour at room temperature then transferred to the blocked microfuge tube and mixed with biotinylated MSLN antigen. In subsequent rounds, ~3×10¹¹ of input phage from each round of amplified phage was used. In the first round 1 ug of biotinylated MSLN antigen was used. In subsequent rounds both 1 ug and 100 ng of antigen were used. After an hour incubation at room temperature, the input phage/biotinylated MSLN mixture was transferred to the other blocked streptavidin well and incubated for 30 minutes at room temperature. This was followed with wash steps {3×300 μL PBS-T (PBS+0.05% Tween 20) (quick); 2×300 μL PBS-T+ (PBS+0.05% Tween 20) (incubate 5 minutes each wash); 3×300 μL PBS (quick); 2×300 μL PBS (incubate 5 minutes each wash)} and elution with 100 μL of 100 mM TEA. Phage were then removed from wells and neutralized with 50 μL of 1M Tris-HCl PH 7.4 in a new tube. 3 mL of exponentially growing TG1 E. coli culture previously grown at 37° C., 250 rpm, until OD₆₀₀=0.5 in 2YT+2% glucose in a 15 mL Falcon tube, was infected with the eluted phage. A 100 μL aliquot of uninfected TG1 E. coli cells was set aside as a control. Eluted phage were Incubated at 37° C. for 30 minutes with no shaking and then an aliquot was used for titer (dilutions of 102 to 108) and plating on 2YT plates overnight at 32° C. The remaining 3 mL of infected TG1 culture, proceeded with overnight phage amplification using M13KO7 helper phage (~1×10¹⁰ cfu).

The next day the eluted titers were calculated to determine the amount of input phage for the subsequent round. The cell culture containing the amplified phage was centrifuged at 5000 rpm, 30 minutes and the supernatant was filtered through 0.22 μM filter unit (Millipore) and precipitated in 20% PEG/2.5M sodium chloride (NaCl) followed by centrifugation and re-solubilization in PBS (pH7.5). Amplified phage titer was determined (dilutions of 10⁴ to 10¹²) in TG1 E. coli cells as grown previously. After 4 rounds of panning, the sequences of positive colonies from phage ELISA were analyzed.

LPAR1 Library-II with an approximate size of 2×10⁷ was phage-recued and the phage titer of 1.0×10¹⁰ cfu/uL was used to pan against recombinant MSLN-His (passive absorption) and in vitro biotinylated MSLN antigen. Four rounds of panning was performed in either approach. For passive panning, with alternating blocking buffers [e.g. Starter Block (Thermo Fisher Cat #37559) for rounds1, 3 and biotin-free casein for rounds 2, 4. Panning was alternated between both Pierce™ streptavidin coated wells (Round 1, 3) (Thermoscientific cat #15501; lot #TF252884) and Pierce™ neutravidin coated wells (Round 2, 4) (Thermoscientific cat #15508; lot #SK253835). One neutravidin well was rinsed with 100 μL PBS and coated with 1 μg of biotinylated human BCMA-FC5 (well #3) and second well (negative control) (well #1) was filled with the PBS only and the plate was incubated at 37° C. for 1 hr. Additionally, one well (well #2) in an Immulon 4HBX plate was also coated with 5 μg FC5VHH (the llama fusion protein) and incubated for 1 hr at 37° C. All three wells were blocked with Starting block for 1 hr at 37° C. and then rinsed with 300 μL PBC.

Phage Library input phage (~1×10¹²) was added to the well #1 and incubate 1 hr at room temperature. The input phages (supernatant of well #1) were transferred to the well #2 (Immulon 4HBX plate) and incubated for an additional 1 hr at room temperature. The phage supernatant were then transferred to the antigen well (well #3) and incubated for 1 hr at room temperature. This was followed with wash steps {3×300 μL PBS-T (PBS+0.05% Tween 20) (quick); 2×300 μL PBS-T+ (PBS+0.05% Tween 20) (incubate 5 minutes each wash); 3×300 μL PBS (quick); 2×300 μL PBS (incubate 5 minutes each wash)} and elution with 100 μL of 100 mM TEA. Phage were then removed from wells and neutralized with 50 μL of 1M Tris-HCl PH 7.4 in a new tube. 3 mL of exponentially growing TG1 E. coli culture previously grown at 37° C., 250 rpm, until OD₆₀₀=0.5 in 2YT+2% glucose in a 15 mL Falcon tube, was infected with the eluted phage. A 100 μL aliquot of uninfected TG1 E. coli cells was set aside as a control. Eluted phage were incubated at 37° C. for 30 minutes with no shaking and then an aliquot was used for titer (dilutions of 102 to 108) and plating on 2YT plates overnight at 32° C. The remaining 3 mL of infected TG1 culture, proceeded with overnight phage amplification using M13KO7 helper phage (~1×10¹⁰ cfu).

The next day the eluted titers were calculated to determine the amount of input phage for the subsequent round. The cell culture containing the amplified phage was centrifuged at 5000 rpm, 30 minutes and the supernatant was filtered through 0.22 μM filter unit (Millipore) and precipitated in 20% PEG/2.5M sodium chloride (NaCl) followed by centrifugation and re-solubilization in PBS (pH 7.5). Amplified phage titer was determined (dilutions of 10⁴ to 10¹²) in TG1 E. coli cells as grown previously. The panning was repeated for three more rounds as described above but the washing conditions was more stringent as described elsewhere (Baral T N et al 2013). In subsequent rounds, ~1×10¹² of input phage from each round of amplified phage was used.

After 4 rounds of panning, the sequences of positive colonies from phage ELISA were aligned and analyzed to identify the unique VHH sequences.

### Results

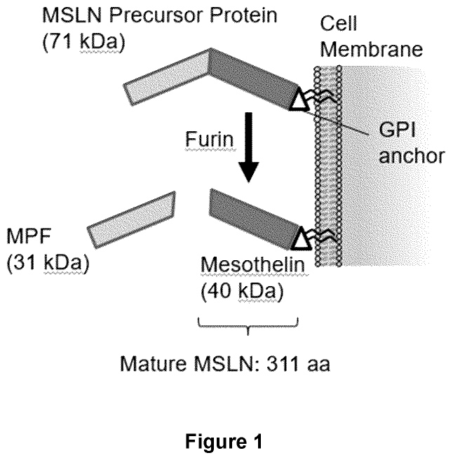

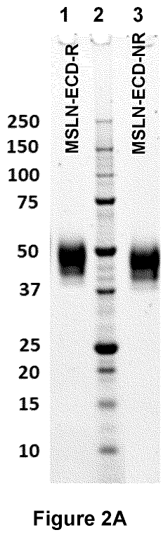

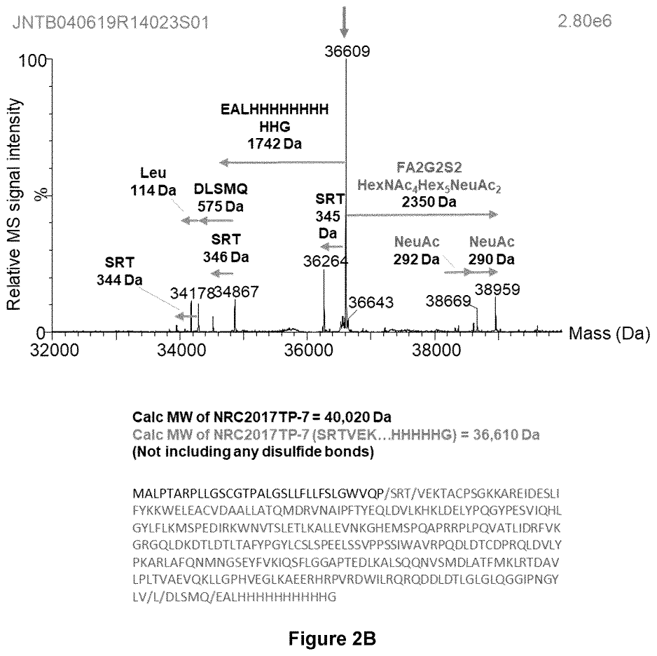

The extracellular domain of human MSLN which include a 309 amino acids (amino acids 2 to 310 of mature MSLN/UniProt Q13421) (FIG. 1) with a 36 amino acid leader signal and 10×His-tag was cloned into pTT5 mammalian expression vector. The MSLN protein was expressed in CHO cells (NRC-HHT Montreal, Protein Production Team) and purified by Immunoaffinity chromatography (IMAC) and analyzed on SDS-PAGE under DTT-reducing conditions (FIG. 2A) and by mass spectrometry following treatment with PNGaseF (FIG. 2B).

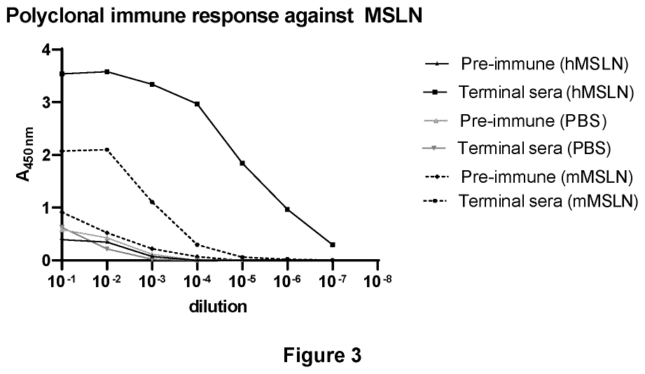

The recombinant MSLN protein was used to immunize a llama (LPAR1) along with some additional proteins (CD22 and BCMA) and the llama immune response was monitored and analyzed by ELISA using MSLN protein as the coating antigen. As shown in FIG. 3, MSLN-ECD injection elicited a strong heavy chain immune response in llama when it compared with the other two antigens used in immunization. The heavy chain immune response in llama's serum is measured by the use two monoclonal antibodies (mAbs; NRC in-house; unpublished results) which specifically bind to the heavy chain IgG2 and IgG3 llama sub-classes.

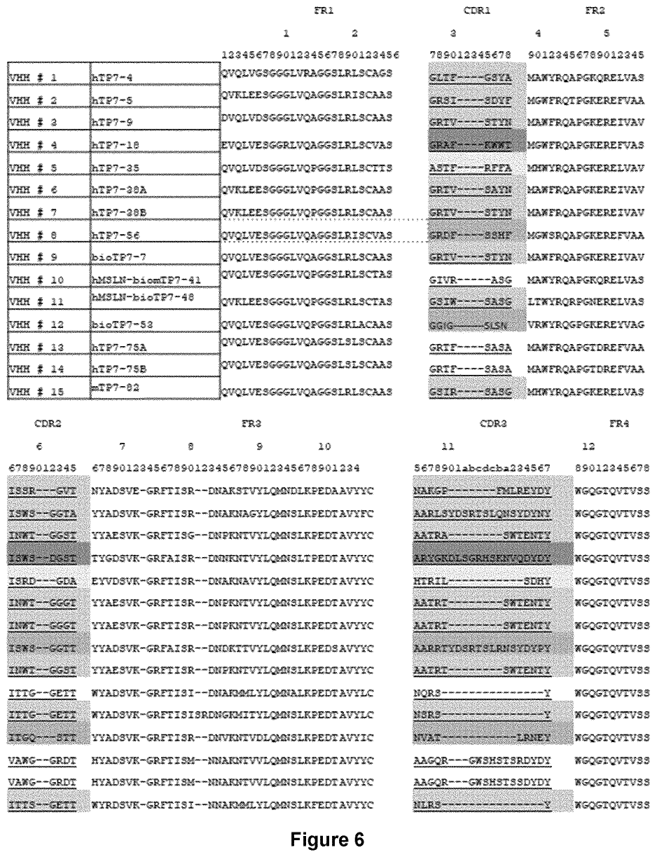

The heavy chain repertoire of llama immunoglobulins was amplified by gene-specific primers and cloned into a phagemid vector (pMED1). A medium size library (2×10⁷) was constructed and its complexity was analyzed by sending 96 colonies for sequencing. The sequencing data showed that the library has high complexity as all the VHH sequences were full-length with no repeating sequences. The library was phage-rescued using M13 helper phage as described elsewhere (Baral T N, Mackenzie R, Arbabi Ghahroudi M. Single-domain antibodies and their utility. Curr Protoc Immunol. 2013 Nov. 18; 103:2.17.1-2.17.57) and the phage antibodies were used in panning experiments with two separate approached of passive and biotinylated panning where the recombinant MSLN was passively absorbed on a well of an ELISA plate or the biotinylated MSLN was captured on immobilized streptavidin plate. After four rounds of panning, 96 colonies from each panning strategy were grown and superinfected by M13 helper phage as described elsewhere (Baral T N et al 2013) and the phages were used in ELISA. Positive colonies were sent for sequencing and the sequencing data were analyzed. Alignment of the sequences was done using OPIG software and IMGT numbering (see FIG. 6). Based on the table provided, 13 unique VHH sequences were selected for gene synthesis and cloning into an NRC in-house expression vector (pMRO).

FIG. 1 depicts the processing of human MSLN (known also as Pre-pro-megakaryocyte-potentiating factor or CAK1 antigen) molecule from its 69-kDa precursor protein on the cell surface by endoprotease furin. MSLN isoform 1 is the predominant isoform with 309 aa and is expressed at low level on the surface of mesothelial lining the pleura, pericardium, and peritoneum. However, it expresses at aberrantly high level in several cancers including mesothelioma, ovarian, pancreatic, and lung cancers and is a promising target for solid tumor immunotherapy (Ma J et al 2012, JBC 287, 40, 33123-33131).

FIG. 2A) depicts an SDS-PAGE of IMAC-purified human isoform I MSLN extracellular domain chain (309aa+10×His-tag) under reducing and non-reducing conditions; B) Mass spectrometry data showed that the MSLN protein mass is consistent with the expected molecular weight (36.3 kDa) and is largely intact with some level of glycosylation which is reflected on the protein gel.

FIG. 3 depicts the llama polyclonal immune response from a pre-immune test bleed (day 0) and the final bleed (7 days post 5th immunization) against hMSLN protein. The mouse recombinant MSLN (mMSLN) (~58% identity with hMSLN) produced in a similar method at the NRC-HHT was used in ELISA and the results showed that there is a weaker immune response against the mMSLN.

### Discussion

The extracellular domain of the predominant human MSLN isoform 1 was successfully expressed in mammalian CHO and HEK-293 cell systems and the recombinant MSLN-ECD performed well in all downstream analytical assays including mass spectrometry (FIG. 2B). After immunizing a llama with the recombinant MSLN-ECD followed by four additional boosts with the same protein, a strong heavy chain immune response was generated as determined by ELISA using heavy chain-specific mAbs. The immune response against mouse MSLN (around 58% homology with the human ECD-MSLN), which was expressed and purified in a similar manner at the Protein Production Team in HHT-Montreal, was not as strong as the one to the human MSLN which is somewhat expected. By constructing a library on the heavy chain repertoire, it may be possible to isolate VHH domain antibodies cross-reacting to both mouse and human MSLN-ECD.

### Example 2: sdAb Characterization

### Introduction

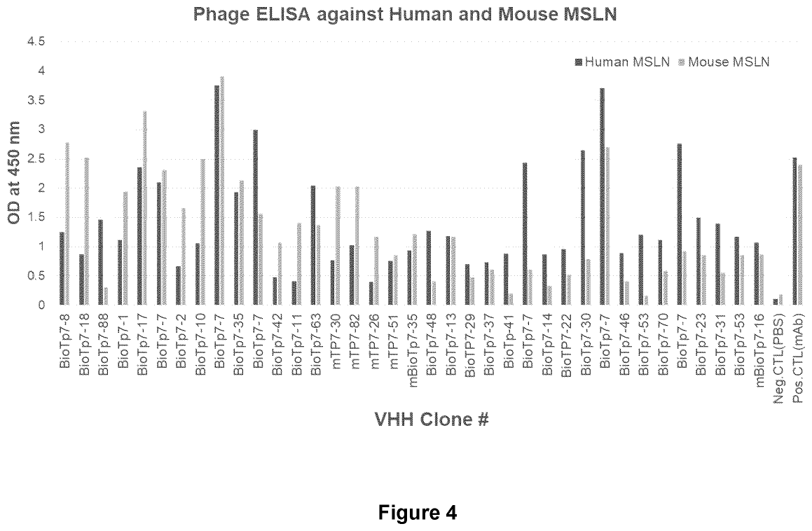

Library construction on the heavy chain repertoire of immunized llama was performed following obtaining a positive immune response against the human MSLN-ECD. More than two hundred individual colonies were screened by phage-ELISA after performing passive and biotinylated panning strategy where either recombinant MSLN or its chemically biotinylated form were captured on a Nunc ELISA wells or on a streptavidine/neutravidin surface, respectively. The captured immunogen was then exposed to the rescued VHH-phages of the immune library. After four rounds of panning, individual colonies were screened by phage ELISA for binding to hMSLN/mMSLN from either panning strategy (FIG. 4). Positive VHH clones were sequenced and grouped based on their CDR1-3 sequences, resulting initially in 13 unique VHH sequences. In certain subsequent experiments, variants of the VHHs named hMSLN-TP7-38A and hMSLN-TP7-75A were identified and used, for a total of 15 unique VHHs. These variants are named hMSLN-TP7-38B and hMSLN-TP7-75B, respectively.

### Materials and Methods

### Expression of Soluble V_{H}H

The DNA sequences of the most repeated clones with phage ELISA OD₄₅₀>0.8 were sent for Gene synthesis to TWIST Bioscience and subsequently cloned into pMRO (a pET28a derivative, Novagen) expression vector. E. coli BL21 (DE3) cells were transformed with the VHH constructs and the respective clones were grown in 0.25-liter cultures of 2×YT medium+ampicillin (100 mg-mL-1) with 0.1% glucose to an OD600 of 0.8. Cultures were induced with 1 mM IPTG and grown overnight on a rotary shaker at 37° C. After confirming of expression by SDS-PAGE and Western blotting, recombinant VHH proteins were extracted from the bacterial cells by standard lysis methods and purified by immobilized metal affinity chromatography (IMAC) and quantified as described elsewhere (Baral & Arbabi-Ghahroudi 2012). The VHH proteins were run on a Supdex 75 Size exclusion chromatography and the monomeric fractions were collected for further SPR analysis.

### SPR Analysis

For surface Plasmon resonance, as mentioned above selected VHHs were passed though size exclusion columns, Superdex 75 (GE Healthcare), respectively, in 10 mM HEPES, pH 7.4, containing 150 mM NaCl, 3 mM EDTA, and monomeric sdAb fractions were collected and protein concentrations were determined by measuring absorbance at 280 nm (A280). SPR analysis were performed with Biacore T200 instrument (GE Healthcare). All measurements were carried out at 25° C. in 10 mM HEPES, pH 7.4, containing 150 mM NaCl, 3 mM EDTA and 0.005% surfactant P20 (GE Healthcare). Approximately 500 RUs of the recombinant monomeric human and mouse MSLN-ECD (obtained after SEC purification of the MSLN-ECD) were captured on SA sensor chip (GE Healthcare) at a flow rate of 5 uL/min. Various concentration of the monomeric VHHs (20-500 nM) were injected over MSLN-ECD surface, respectively using an SA surface as a reference at a flow rate of 40 μL/min. Surfaces were generated by washing with running buffer. Data were analysed with BIAevaluation 4.1 software.

### Epitope Binning by SPR

In addition to obtaining binding kinetic data, Biacore co-injection experiments were performed on selected anti-MSLN VHHs to determine whether these antibodies could bind unique or overlapping epitopes on MSLN-ECD protein surface. Briefly, 80 μL of the first VHH diluted in HBS-EP buffer to a concentration of 5 times its KD value and was injected over 500 RUs of immobilized MSLN-ECD at 40 μL/min. Following injection of the first VHH, buffer or a second VHH (80 μL total volume, at 5×KD) was injected at 40 μL/min over the MSLN-ECD surface already saturated with the first VHH. Data were collected on all possible paired combinations of VHHs, in both orientations (i.e. each VHH acted as the first and second VHH) and evaluated as described above.

### Evaluating Target Specificity of Anti-MSLN VHHs by Mirrorball™ Microplate Cytometry

Binding to MSLN-positive and MSLN-negative tumor cell lines was assessed using a Mirrorball® high-sensitivity microplate cytometer (TTP Labtech Inc, Melbourn, UK). MSLN-positive NCI-H292 (NCI-H292; Human Lung cells) and Mesothelin Negative NCI-H1581 cells. Start up each cell line from liquid nitrogen frozen stock and grow in T-25 flask until 85% confluent at 37° C. in a humidified atmosphere containing 5% CO₂. Cells were dissociated in Accutase® solution (Sigma-Aldrich), washed with Hank's Balanced Salt Solution (Thermo Fisher) and counted. Cells (5000 cells well-1) were seeded in Nunc® MicroWell 96-well optical bottom plates (Sigma-Aldrich) and incubated at 37° C. in a humidified atmosphere containing 5% CO₂ for 24-48 h. Each anti-MSLN biotinylated VHH was serially diluted in Live Cell Imaging Solution (LCIS; Thermo Fisher) and 50 μL of each Ab variant was added to the assay plate and incubated at 4° C. for 2 h. As positive control, anti-MF-T (human IgG1) monoclonal antibody (NRC-HHT-Montreal) was used. The wells were washed with LCIS, then Biotin-Streptavidin, R-Phycoerythrin Conjugate (SAPE) or donkey anti-Human Alexa Fluor (AF488) (at 3 ug/ml final concentration) was added to the wells and incubated at 4° C. for 1 h. The secondary Ab was removed, the cells were washed with LCIS then 50 μL of DRAQ5 nuclear stain (1 μM prepared in LCIS; Cell Signaling, Danvers, MA, USA) was added to wells and incubated for 10 min at 4° C. The assay plate was read using the following settings: (i) laser settings, 488 and 640 enabled, 6.0 mW; (ii) channel settings, FL-2 (488-540 nm), voltage 550 V, sensitivity 2, “Tiff files saved” FL-3 (560-610 nm), voltage 575 V, sensitivity 2, “Tiff files saved” and FL-4 (650-690 nm), voltage 550 V, sensitivity 3, trigger, “Tiff files saved”; (iii) object characteristics, FL-2, FL-3 and FL-4 (peak intensity, mean intensity, median intensity and baseline); (iv) population definition, objects-cell filters (FL-4 perimeter range 20-1000 nm); and (v) population statistics, number of cells, mean (FL-2 mean intensities), median (FL-2 mean intensities), mean (FL-3 mean intensities), median (FL-3 mean intensities), mean (FL-4 mean intensities) and median (FL-4, mean intensities). Data were analyzed using Cellista software (TTP Labtech) and GraphPad Prism 6 software. Curves were fit to the data using a one-site-specific binding with Hill slope model.

### Evaluating Target Specificity of Anti-MSLN VHHs by Flow Cytometry

Purified VHHs were used to assess the target specificity the cell line surface by flow cytometry. The highly MSLN expressing human ovarian cancer cell line SKOV3 was used to assess whether purified MSLN-specific VHH showed binding to human cells. SKOV3 cells were incubated with various MSLN-VHH proteins over a 5 fold dilution series from 5-0.007 μg/mL. The binding of the MSLN-targeted VHH to cell surface MSLN was detected by flow cytometry using a mixture of two secondary mouse antibodies with broad reactivity to llama VHH proteins conjugated with AlexaFluor647. Following secondary antibody labelling, cell binding was assessed using flow cytometry.

### Results

The panning of the immunized llama VHH against both passively absorbed and biotinylated human and mouse MSLN resulted in multiple specific VHH binders as shown in FIG. 4 by phage ELISA. The sequencing results identified 15 unique VHH sequences from both panning strategies (Table 1). It is noted that the CDR sequences of the antibodies named hMSLN-TP7-38A and hMSLN-TP7-38B are identical to each other except for an Ala→Thr substitution at position 6 of CDR1 of hMSLN-TP7-38B as compared to hMSLN-TP7-38A. It is further noted that the CDR sequences of the antibodies herein termed hMSLN-TP7-75A and hMSLN-TP7-75B are identical to each other except for an Arg→Ser substitution at position 13 of CDR3 of hMSLN-TP7-75B as compared to hMSLN-TP7-75A. Based on these sequence features and ensuing results, it is clear that a degree of sequence variation in the CDRs is tolerated.

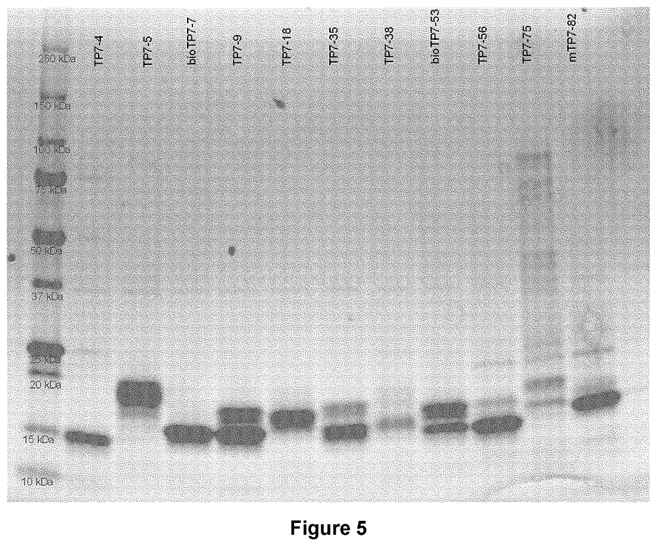

The gene synthesis and sub-cloning of all 15 anti-MSLN VHH was performed by TWIST Bioscience (USA) and the pMRO plasmid DNA were transformed into BL21 (DE3) E. coli for protein expression. The presence of a Histidine tag and biotinylation signal sequence (Avitag™) in the pMRO vector allows facile purification by IMAC column as well as specific addition of a biotin moiety at the VHH C-terminal. The single biotin addition facilitates VHH detection in future epitope mapping and other cell-based assays. The IMAC-purified VHH proteins were run on a SDS-PAGE (FIG. 5). As shown, the VHH antibodies showed an expected molecular weight of around 15-17 kDa.

The state of aggregation of the purified protein was checked by size exclusion chromatography (SEC) and as expected all were non-aggregating monomers (data not shown). The reactivity of the individual VHH protein was also confirmed by ELISA in which rabbit anti-His6 antibody conjugated to HRP was used for the detection of VHH binding to the immobilized MSLN-ECD (data not shown).

The monomeric fraction of VHHs obtained by SEC purification were used for SPR experiment where the human or mouse MSLN-ECD was immobilized onto the CM5 dextran chip and various VHH concentration (20-500 nM) were passed over the sensor chip. SPR analysis revealed all tested VHHs specifically bound MSLN-ECD with equilibrium constants ranging from 276 nM for TP7-18 to 58 PM for mTP7-82. All of the data collected fit a 1:1 binding model (Table 3A). SPR data on mouse MSLN-ECD showed that only three VHHs (TP7-4, bioTP7-7 and mTP7-82) show mouse cross-reactivity with an affinity between 145-900 nM. The remaining VHHs showed no significant binding to the mouse MSLN (Table 3B).

For epitope binning of anti-MSLN VHHs, co-injection SPR experiments were performed with pairs of VHHs in both orientations to determine if antibodies could bind MSLN-ECD simultaneously. If there is an increase in response upon co-injection of any two VHHs, this will indicate that binding of the first VHH (at saturation concentration) does not hinder the binding of the second one and, therefore, these antibodies recognize independent epitopes. However, if there is a minor change in response upon co-injection of two VHHs, this will indicate that the two VHHs could not bind simultaneously to the same region and, therefore, they recognize overlapping/identical epitopes. The co-injection SPR experiments were performed on anti-MSLN VHHs and identified four bins with distinct epitopes. Bin 1 include six VHHs, Bin 2 one VHH, Bin three one VHH and Bin 4 four VHHs. Epitope binning was not determined for TP7-75 (Table 3A).

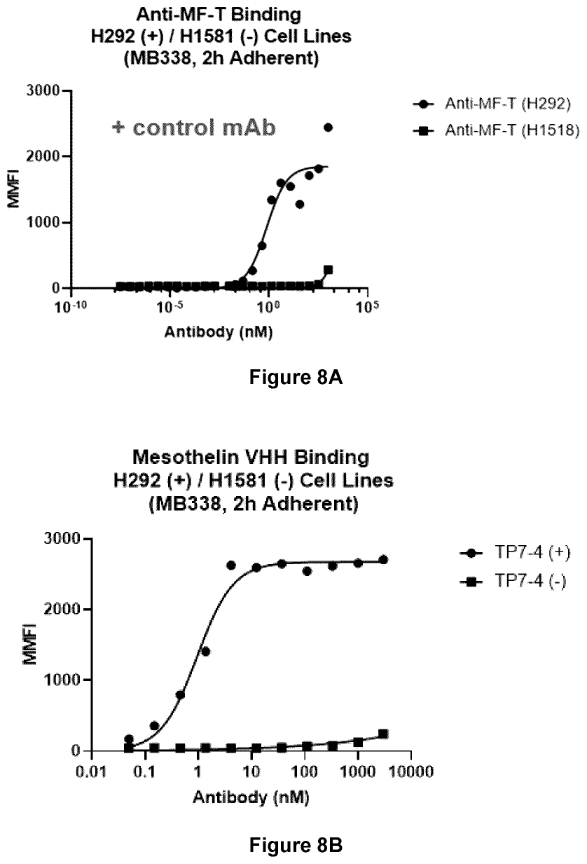

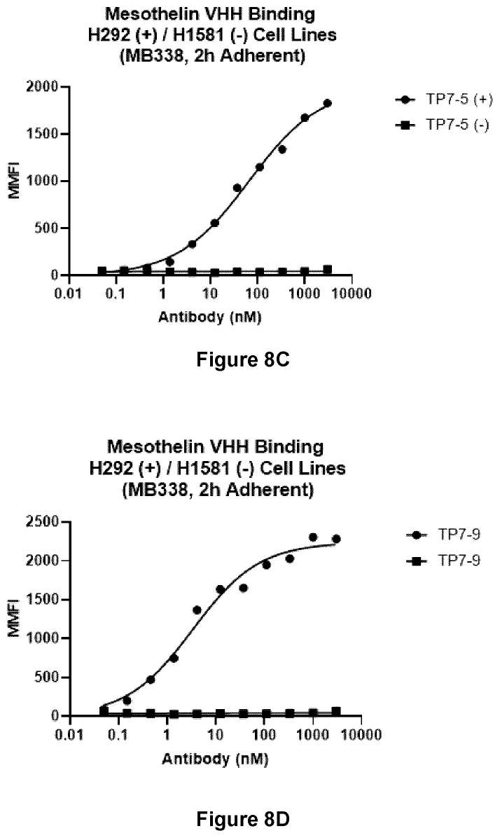

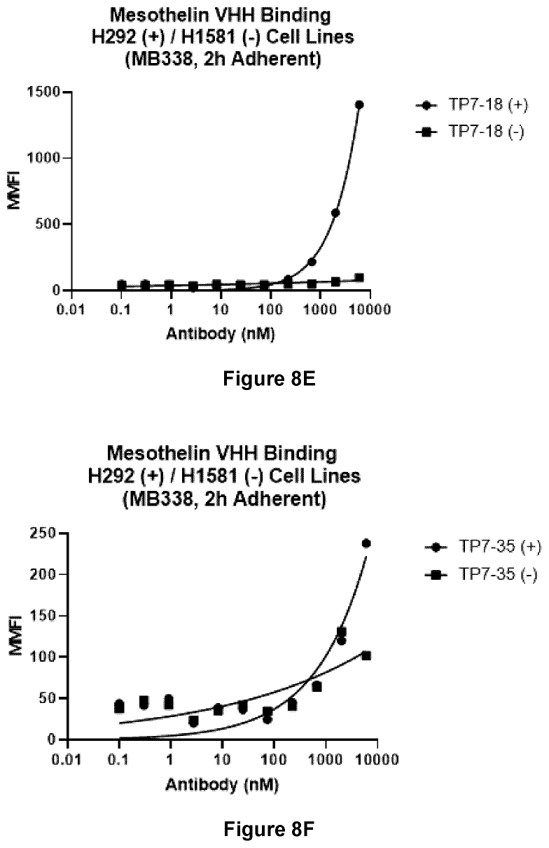

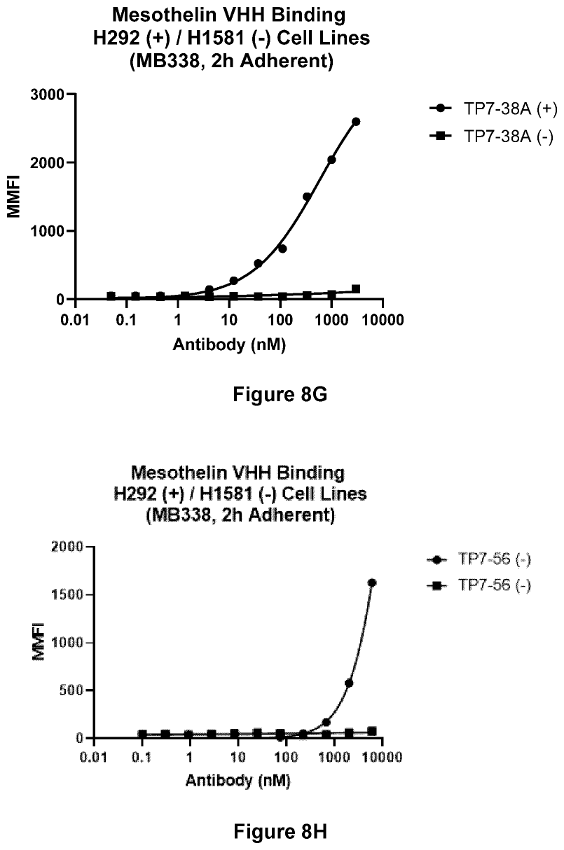

Cell binding activities of the selected VHHs were examined by Mirrorball where negative-(NCI-H1581) and positive (NCI-H292)-MSLN cells were cultured in 96 well plate and binding of the biotinylated VHHs was detected by Streptaviding-PE conjugate. As positive control, a human IgG1 mAb MF-T was used which was detected by donkey anti-human AF468. As shown in FIGS. 8A to 81, TP7-4, TP7-5, TP7-9, TP7-38, TP7-56, and TP7-75 VHHs show cell binding activities to MSLN-positive cells and no significant binding to MSLN-negative cells.

Assessment of MSLN-sdAb cell binding via flow cytometry demonstrated very high cell binding for BioTP7-7, TP7-82, TP7-4, and TP7-9. Detectable but much lower cell binding was observed for TP7-38, TP7-56, TP7-5, TP7-18, and TP7-56. No significant cell binding was observed for TP7-35, TP7-75, TP7-82, and TP7-69.

FIG. 5 depicts the SDS-PAGE of 11 anti-MSLN VHH antibodies expressed in BL21 (DE3) E. coli and purified by IMAC. The purified proteins showed expected molecular weight of 15-17 kDa and there was no sign of degradation in all protein samples. The expression data for two VHHs: biomTP7-41 and bioTP7-48 has not be shown.

Table 1 depicts the amino acid sequences of 15 VHHs. The CDR regions are underlined and Framework regions are numbered according to IMGT numbering system.

Tables 3A and 3B depicts the measured affinities of VHHs against human and mouse MSLN, respectively. The affinities data for human MSLN range from 276 nM for TP7-18 to 57 PM for mTP7-82 and for the mouse MSLN range form 145-900 nM.

TABLE 3A
Measured affinities of VHHs against human MSLN

| | Old | | | kd | KD |
|---|---|---|---|---|---|
| Clone No.-Bin | name | Target | ka(1/Ms) | (1/s) | (M) |
| VHH # 1-Bin1 | TP7-4 | hMSLN | 1.72E+06 | 3.88E−04 | 2.25E−10 |
| VHH # 2-Bin1 | TP7-5 | hMSLN | 2.29E+05 | 4.41E−03 | 5.72E−08 |
| VHH # 3-Bin1 | TP7-9 | hMSLN | 1.78E+06 | 2.38E−03 | 1.34E−09 |
| VHH # 4-Bin2 | TP7-18 | hMSLN | 6.62E+02 | 1.38E−02 | 1.29E−08 |
| VHH # 5-Bin3 | TP7-35 | hMSLN | 1.66E+04 | 8.11E−04 | 2.10E−08 |
| VHH # 6-Bin1 | TP7-38A | hMSLN | 2.45E+06 | 3.48E−02 | 3.15E−08 |
| VHH # 7-Bin4 | TP7-56 | hMSLN | 7.57E+05 | 2.01E−02 | 2.65E−08 |
| VHH # 8-Bin1 | bioTP7-7 | hMSLN | 3.80E+06 | 3.51E−04 | 9.24E−11 |
| VHH # 9-Bin 4 | biomTP7-41 | hMSLN | — | — | 2.14E−07 |
| VHH # 10-Bin 4 | bioTP7-48 | hMSLN | — | — | 2.76E−07 |
| VHH # 11-Bin4 | bioTP7-53 | hMSLN | 7.58E+05 | 4.33E−04 | 5.71E−10 |
| VHH # 12-Bin5 | TP7-75A | hMSLN | 5.87E+05 | 3.05E−02 | 5.20E−08 |
| VHH # 13-Bin1 | mTP7-82 | hMSLN | 3.04E+06 | 1.76E−04 | 5.79E−11 |

TABLE 3B
Measured affinities of VHHs against mouse MSLN

| Clone No.-Bin | Old name | Target | KD (M) |
|---|---|---|---|
| VHH # 1-Bin1 | TP7-4 | mMSLN | 300 nM |
| VHH # 8-Bin1 | mTP7-82 | mMSLN | 145 nM |
| VHH # 13-Bin1 | bioTP7-7 | mMSLN | 218 nM |

FIG. 6 depict an alignment of amino acid sequences of all 15 anti-MSLN VHHs.

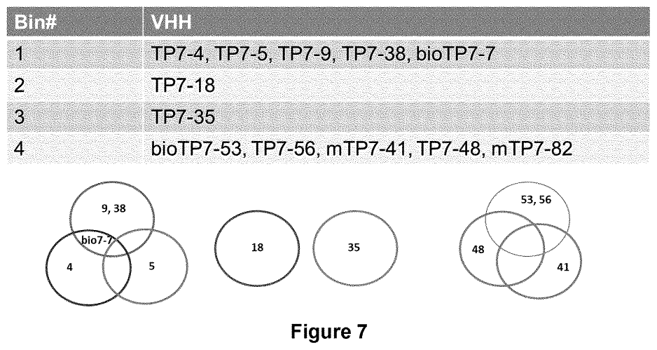

FIG. 7 depicts competitive binding and epitope binning data by SPR.

FIGS. 8A to 8I depicts the cell binding of anti-MSLN VHHs to H292 MSLN-positive cell lines. As negative control, MSLN H1581 cell line was used. Human IgG1 MF-T anti-MSLN mAb was used as positive control antibody. TP7-4, TP7-5, TP7-9, TP7-38, TP7-43, TP7-56, TP7-56 TP7-75 show cell binding activities and TP7-69 showed non-specific binding to both cell lines.

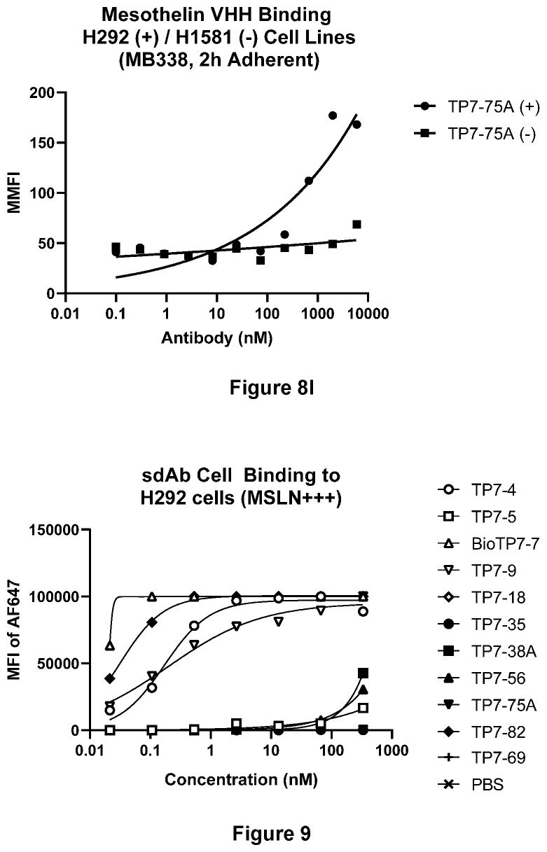

FIG. 9 depicts binding of anti-MSLN VHH to MSLN-high SKOV3 cells as assessed using flow cytometry. Results demonstrate very strong cell binding for some VHH proteins, with weaker or undetectable cell binding for other purified VHH proteins. Low or no cell binding was observed for TP7-35, TP7-75, TP7-82, and TP7-69.

### Discussion

VHH antibodies specific to MSLN were expressed in E. coli and the proteins were purified and biotinylated. The antibodies showed non-aggregating and monomeric behaviors as determined by size exclusion chromatography. The binding kinetics of 13 VHHs were determined by SPR and the antibodies showed specific binding to human MSLN-ECD with affinities ranging from nM to pM (276 nM for hMSLNbiomTP7-41 to 58 PM for mTP7-82). This diverse set of affinities allows us to study the effect of affinity in productivity of CAR-T construct. Epitope binning of VHHs by SPR indicates that the VHHs bind to four distinct and non-overlapping epitopes and that will allow us to examine the effects of VHHs in each Bin in functional cell-based studies. The possibility of competing VHHs to neighboring region could not be ruled out by SPR epitope binning. However, using a collection of various fragments human MSLN ecto-domain expressed as yeast surface display, it is possible to physically map out two epitopes that are differentially required for the binding of sdAbs. The cell binding activates of the VHH was the final essential step towards application of these VHHs in CAR and bispecific constructs. The majority of VHH isolated showed good cell binding activities when compared with a positive mAb control antibodies and showed minimal or no binding to the MSLN-negative cell line. Confirmation of cell binding via flow cytometry demonstrated very low apparent Kd (subnanomolar) for some constructs, while others showed detectable but much lower cell binding capacity. Importantly, different therapeutic strategies may require different affinity, and thus identifying a range of single domain antibodies with a range of affinity was intended here. These results pave the way to use VHH from each bin in the CAR and bispecific constructs and examine their effectiveness in such applications.

### Example 3: CAR-T In Vitro Testing

### Introduction

After identifying novel MSLN-binding single domain antibody (sdAb) sequences described above, it was desired to test their activity within the context of chimeric antigen receptor (CAR) molecules, which can be used to redirect human T cell responses towards cells bearing specific surface antigens. Thus, using high throughput techniques previously described (Bloemberg 2020) novel MSLN-sdAb targeted CAR constructs were generated and tested their relative T cell activating activity via various assays described below.

### Materials and Methods

Single domain antibody antigen binding sequences (ABD) were transferred to a modular CAR plasmid backbone (as per Bloemberg et al 2020) containing restriction sites to allow efficient recombination wherein the antigen binding domain could be removed and replaced with the novel MSLN-sdAb antigen binding domain (ABD) sequences. Specific CAR design used was as follows: Human CD28 signal peptide (SEQ ID NO: 68), sdAb (any one of SEQ ID NOs: 46 to 60), flexible linker domain (SEQ ID NO: 69), human CD8 hinge domain (SEQ ID NO: 70), human CD8 or CD28 transmembrane domain (SEQ ID NO: 71 or 72), human 4-1BB signal transduction domain (SEQ ID NO: 73), and human CD3-zeta signal transduction domain (SEQ ID NO: 74). Exemplary MSLN CAR sequences are shown in SEQ ID NO: 61 to 66. Primers used to transfer VHH sequences into the universal CAR backbone resulted in the conversion of the N-terminal sequence for all MSLN-CARs to QVQLVE, but the application of other canonical N-terminal sequences can also result in functional CARs. With use of degenerate primers, the third position of the FR4 region could switch from L to Q and vice versa.

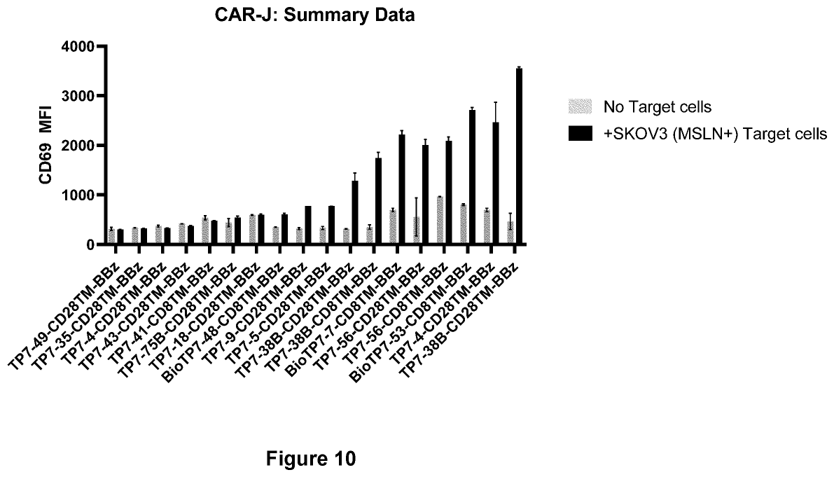

Novel MSLN-targeting CAR constructs were then tested for activity in an immortalized human T cell line (Jurkat) similarly as described in Bloemberg 2020. In brief, plasmids were electroporated into Jurkat T cells and allowed to recover for several hours. Jurkat-CAR cells were then cultured with or without a target cell line positive for expression of human MSLN (SKOV3). In order to quantitate CAR-mediated Jurkat cell activation, expression of CD69 was measured using specific antibody staining and flow cytometry. Using expression of GFP-marker to gate CAR-expressing cells, the level of T cell activation as determined using the CD69-surface marker was clearly elevated in various Jurkat cells expressing various MSLN-sdAb targeted CAR constructs when cells were placed in co-culture with MSLN expressing SKOV3 cells but not with Jurkat cells alone (FIG. 10). Similar experiments were performed with cell lines devoid of MSLN expression (Raji, H1581) to confirm lack of activation with these target lines (data not shown).

Following this CAR-J testing, several MSLN-CAR constructs were selected for testing in primary human T cells (TP7-5, TP7-9, TP7-38, and TP7-56). To accomplish this, lentivirus was prepared through co-transfection of CAR plasmids with lentiviral packaging cell lines. Lentiviral particles in the cell supernatant were collected and concentrated using ultracentrifugation. Primary human T cells were then isolated from a healthy donor blood samples using magnetic bead separation and polyclonally activated using anti-CD3 and anti-CD28 beads. Activated human T cells were then transduced with concentrated lentivirus containing various MSLN-targeted CAR constructs at pre-determined multiplicity of infection. Following viral transduction, cells were confirmed to express CAR using flow cytometric analysis for GFP-marker. Virally transduced T cells (CAR-T cells) were then expanded for 9 days before examination for CAR activity.

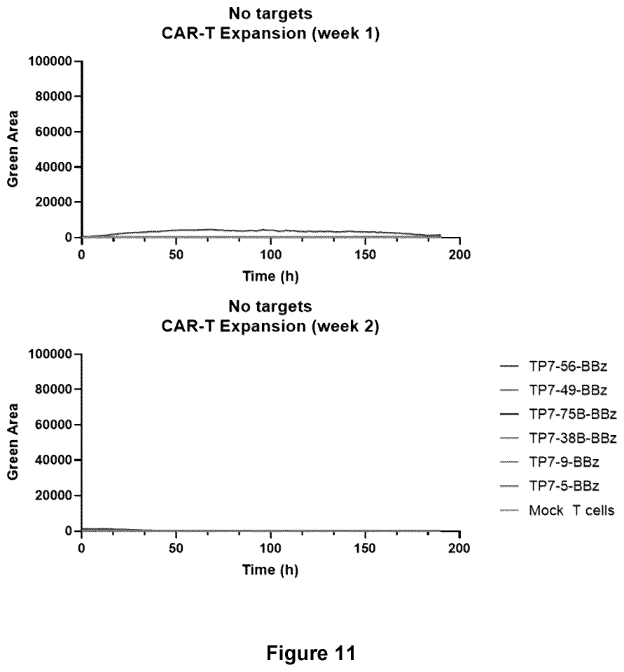

To examine CAR activity in virally transduced CAR-T cells a number of assays were utilized. Firstly, cells were placed without additional stimulation in controlled cell culture conditions and examined for non-specific cellular expansion over an additional 14 days via live microscopy using an IncuCyte® S3 device (Sartorius, USA). Total cell count was determined using automated cell counting. Primary human T cells stably transduced with various MSLN-sdAb targeted CAR constructs did not show significant cell expansion when left in unstimulated conditions between day 15 and 30 post-polyclonal activation (FIG. 11). These results indicate that MSLN-sdAb targeted CAR constructs tested do not confer strong target-independent tonic T cell activation to primary human T cells.

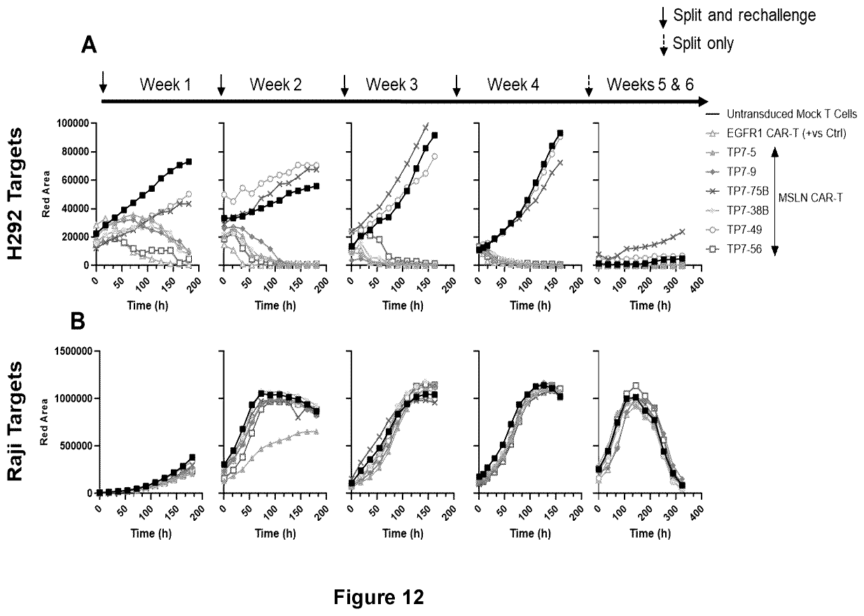

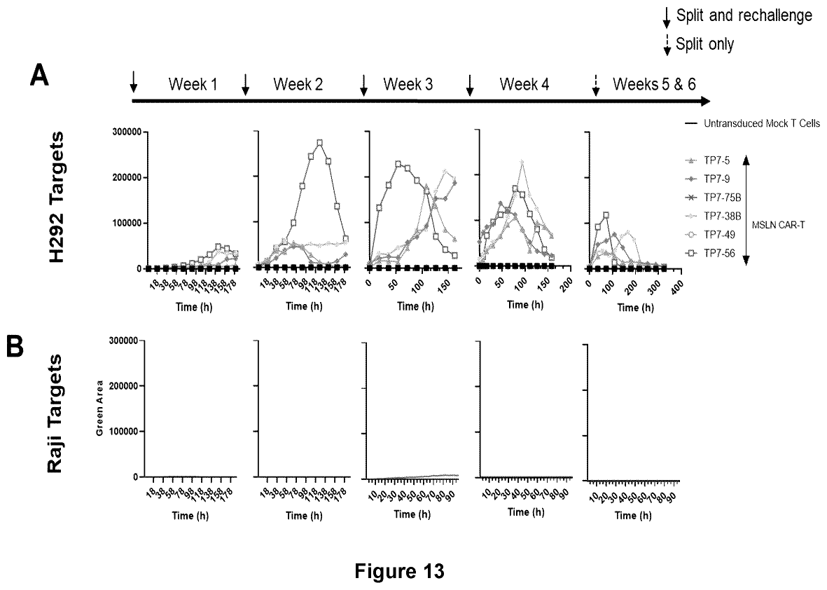

Following this, primary CAR-T cells were tested for antigen specific activation and target cell killing in response to cells with (H292 cells) and without (Raji cells) MSLN expression. CAR-T cells were placed in co-culture with the target cells expressing a red-fluorescent protein tag, NucLight™-Lentivirus (Sartorius, USA), and monitored for long-term co-culture over 6 weeks using the IncuCyte S3 live microscopy device. Media was refreshed, cells were split, and fresh target cells were added weekly. MSLN CAR-T were able to repress target cell growth for the MSLN-positive but not the negative target cell lines (FIG. 12). Examining the number of GFP-labelled CAR-T cells, the MSLN CAR constructs showed clear expansion of GFP+ cells in response to MSLN+ H292 cells but not the MSLN negative Raji cells demonstrating antigen-specific activation and expansion (FIG. 13). These results indicate that MSLN-CAR-T cells can mediate long-term antigen-specific killing of MSLN-expressing target cells.

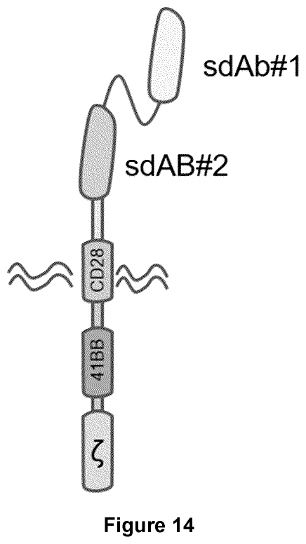

FIG. 14 depicts a model of a tandem CAR construct wherein MSLN-sdAb is combined with an EGFR-specific sdAb. These constructs were generated through DNA synthesis of a construct of design, EGFR-specific sdAb, short linker, MSLN-specific TP7-56 sdAb, human CD8-hinge domain, human CD28-transmembrane domain, human 41-BB domain, and human CD3zeta domain; as per SEQ ID NO: 60. Following synthesis, Tandem-EGFR-MSLN CAR constructs were then utilized for functional testing as below.

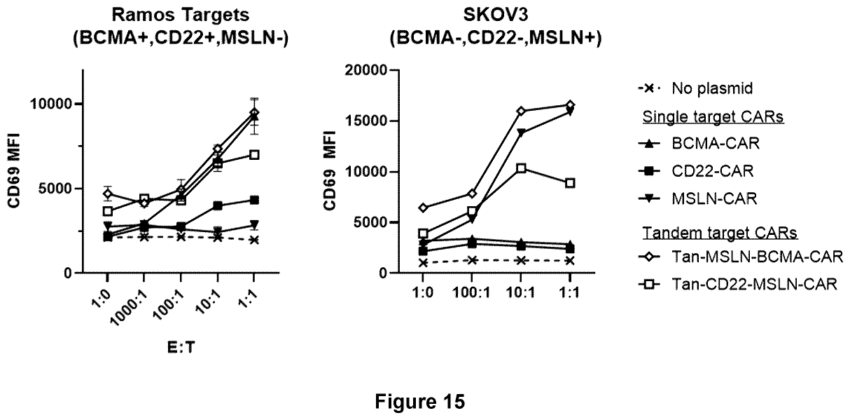

Plasmids containing DNA sequences for EGFR, MSLN-TP7-56, or Tandem-EGFR-MSLN-56 CARs were then electroporated into Jurkat cells, which were co-cultured with EGFR-low/MSLN-low MCF7 target cells or EGFR-high/MSLN-high SKOV3 target cells at varying effector to target cell ratios. After overnight incubation of co-cultures, CAR-Jurkat cells were examined for expression of CD69 using flow cytometry. Results shown in FIG. 15 demonstrate enhanced recognition of EGFR-high/MSLN-high target cells by Tandem-CAR constructs. These results demonstrate that

### Results

FIG. 10 depicts the results of CAR-Jurkat assay wherein Jurkat cells were transiently electroporated with varying CAR plasmids and cultured alone or in co-culture with MSLN-positive SKOV3 cells. The level of T cell activation was measured using human CD69-specific antibody staining and flow cytometry. Graphs depict the mean fluorescent intensity for CD69-staining for each single domain antibody targeted CAR constructs performed in a single experiment in duplicate, either in culture with no target cells (first bar) or with MSLN positive SKOV3 target cells (second bar). Error bars show the standard error of the mean for duplicate wells. Results demonstrate varying levels of antigen-specific responses with the MSLN CAR constructs tested.

FIG. 11 depicts the results of CAR-T tonic activation assay wherein primary donor blood derived T cells were transduced with varying CAR constructs and examined for target-independent expansion. Mock refers to donor derived T cells exposed to similar treatment conditions in the absence of any CAR-expressing lentivirus. As described in methods, CAR-T cells were examined between day 15 and 29 post-polyclonal activation for proliferation in cell culture via live microscopy. Graphs depict the fold change in GFP-marked CAR-T cell number relative to number of cells the start of this assay as determined using automated cell counting. Results demonstrate a lack of antigen-independent T cell expansion in those CAR constructs tested.

FIG. 12 depicts the results of CAR-T target growth repression assay performed using donor blood derived T cells transduced with varying MSLN-single domain antibody or EGFR-specific comparator CAR constructs. Mock refers to unmodified donor derived T cells without CAR expression exposed to similar treatment conditions. As described above, red fluorescent protein (mKate2) marked H292 target cells with MSLN expression or Raji cells with no MSLN expression were examined via live fluorescent microscopy for target cell proliferation when in co-culture with CAR-T cells. Graphs depict the total red fluorescent protein marked target cells as determined using automated counting. Results demonstrate specific repression of MSLN-expressing but not the MSLN negative target cells by MSLN-CAR-T cells. These results also demonstrate that MSLN CAR-T cells can remain active and continue to kill MSLN-positive target cells even after 6-weeks in co-culture.

FIG. 13 depicts the results of CAR-T target-specific activation/expansion assay performed using donor blood derived T cells transduced with varying MSLN CAR-T constructs. Mock refers to unmodified donor derived T cells without CAR expression exposed to similar treatment conditions. As described above, GFP-marked CAR-T cells were examined via live fluorescent microscopy for proliferation in co-culture with target cells with (H292) or without (Raji) MSLN-expression. Graphs depict the total green fluorescent protein signal as determined using automated counting. The MSLN-CAR-T cells showed varying degrees of expansion in response to MSLN positive target cells. Based on this data, a select set of MSLN CAR-T constructs were chosen for downstream testing. These results also demonstrate that MSLN CAR-T cells can remain active and continue to proliferate in response to MSLN-positive target cells even after 6-weeks in co-culture.

FIG. 14 depicts the molecular structure of a single-binder (left) or multi-binder (right) chimeric antigen receptor; for multi-binder CAR constructs a sdAb sequence at the 5′ end of a CAR DNA construct is followed by a linker sequence which can be of varying composition, followed by another sdAb sequence which can be the same or different from the first sdAb sequence included in the sequence, then followed by a similar structure to other CAR molecules [hinge domain, transmembrane domain, signaling domain(s)]. A similar molecule structure can also be used to generate multi-antigen binding CAR constructs wherein a MSLN-sdAb sequence is followed by a linker and then an alternate sdAb sequence targeting a different antigen

FIG. 15 depicts the results of Jurkat cell CAR activation activity assay wherein CAR plasmids with varying single or multi-binder formats were electroporated into Jurkat cells, which were then placed in co-cultures containing BCMA-positive, CD22-positive, and MSLN-negative target cells (Ramos; left), or with BCMA-negative, CD22-negative, and MSLN-positive target cells (SKOV3; right). Graphs depict the average CD69-specific antibody staining of Jurkat cells as measured by flow cytometry after overnight incubation of co-cultures. Error bars present the standard error of the mean over 2 duplicate co-culture wells. Results demonstrate similar MSLN-antigen specific activation of T cells expressing CAR molecules with single MSLN binding elements or tandem MSLN and BCMA or CD22-targeted binding elements.

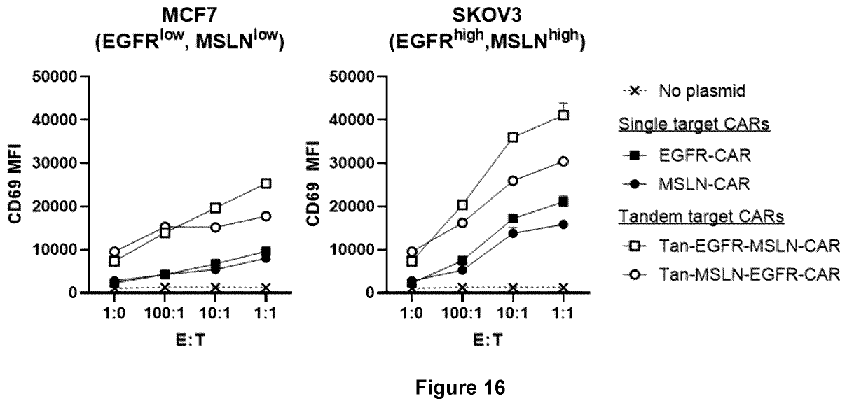

FIG. 16 depicts additive effect of multiple targeting elements combined in a tandem vector. Experiments were performed as described above to examine the effect of expression of CAR proteins targeting EGFR or MSLN, or a tandem CAR combining both. CAR-Jurkat cells were transiently electroporated with various CARs as shown an co-cultured with EGFR-low, MSLN-low MCF7 cells (left), or EGFR-high, MSLN-high SKOV3 cells (right). Results demonstrate that tandem targeting of EGFR and MSLN can result in an additive signaling effect.

### Discussion

Overall, these results exemplify that MSLN-specific single domain binders can generate strong antigen-driven T cell activation signaling which can drive long-term tumour cell growth repression, and CAR-T expansion even after repeated challenges over an extended period of time. While few lead molecules were identified in the exemplary data provided here, molecular optimization may be performed with additional MSLN-specific single domain antibody sequences in order to generate highly functional CAR molecules. As an example of such molecular optimization, data was provide demonstrating that when expressed in multi-binder and/or multi-antigen targeting CAR format, MSLN-constructs maintain strong antigen-specific responsiveness to individual targets, and can provide additive signaling when both targets are present. In addition, combining multiple MSLN-specific single domain antibody sequences in a single molecule may be an effective strategy to increase target-specific CAR activating activity.

### Example 4: CAR-T in vivo Testing

### Introduction

To further confirm the anti-tumor effect of MSLN-binding single domain CAR-T cells in vivo, a xenograft model was established by subcutaneous inoculating H292 tumor cells expressing mKate2 as a reporter into NOD/SCID/IL2r-gamma-chain^{null} (NSG) mice prior to infusion of MSLN-targeting single domain antibody CAR-T cells.

### Materials and Methods

For in vivo studies, female NOD/SCID/IL2Ry^{-/-}(NSG) mice, 6-8 weeks of age, were obtained from Jackson Laboratories and maintained at the Animal Care Facility at the National Research Council of Canada. The mice were housed in pathogen-free individually ventilated cages in a barrier system under conditions. Animals had access to certified rodent diet and sterilized water was given via water bottles. NSG mice lack mature T cells, B cells and natural killer cells; thus, they are better than nu/nu mice for the study. Eight-week-old NSG mice were injected with 6×10⁶ H292-mKate2 cells in 100 L HBSS subcutaneously in the back. On day 3 post tumor cells injection, where palpable tumors were observed in all animals, mice were injected intravenously via the retro orbital plexus with 5×10⁶ single domain MSLN-CAR-T cells, un-transduced mock T cells from the same donor (normalized to the highest CAR-T dose), or with vehicle control. Tumor growth in mice was monitored by twice weekly caliper measurements. Mice were monitored daily for signs of illness and sacrificed immediately if they met pre-specified humane endpoints including but not limited to hind-limb paralysis, respiratory distress, or 30% body weight loss as approved by the Animal Care Committee of the Research Center.

### Results

To assess the activity of MSLN-binding single domain-CAR-T in a xenogeneic model, 8 week old NOD/SCID mice were inoculated subcutaneously with 5×10⁶ H292 tumor cells on day 0, and subsequently treated by retro-orbital injection with 5×10⁶ MSLN-targeted single domain-CAR-T cells (MSLN-TP7-5, TP7-9, TP7-38 or TP7-56) or EGFR-targeted CAR-T cells generated from healthy human donor T cells as described above, or Mock-transduced T cells (no lentivirus) without CAR expression on day 3. Tumor growth was monitored via twice weekly measurement of tumors using digital calipers.

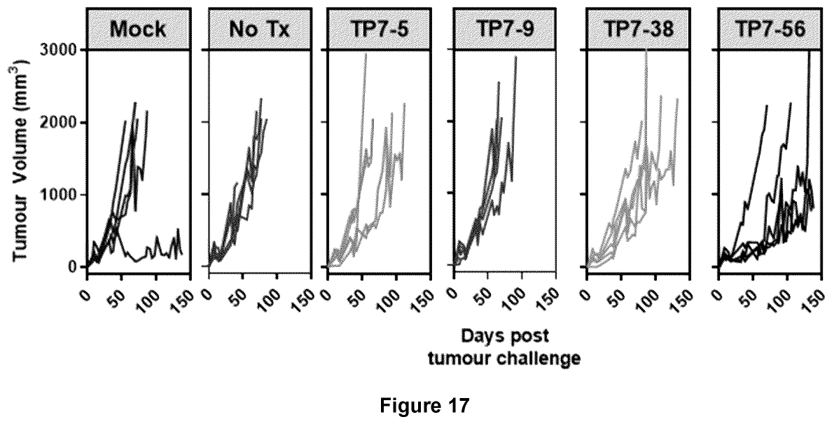

FIG. 17 depicts result of tumor burden in mice that were inoculated with H292 tumor cells and treated with various CAR-T cells. Graphs depicts the tumor growth kinetics over the course of the experiment. Mice treated with MSLN-TP7-56 showed the best tumor growth retardation compared to no treatment or mock T cell treated mice whereas TP7-5 and TP7-38 also showed moderate reduction in tumor burden compared to untreated mice.

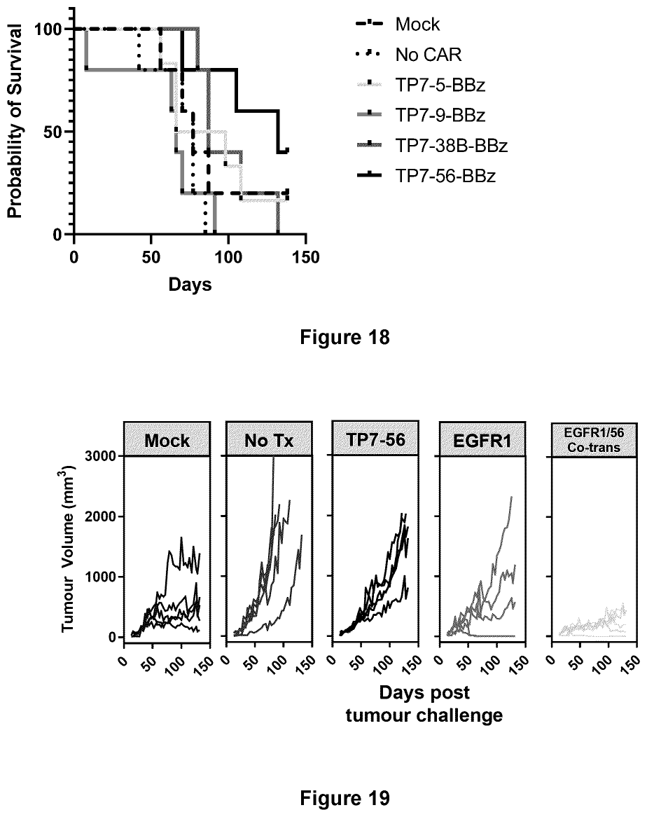

FIG. 18 depicts the proportion of surviving animals in each treatment group throughout the course of the experiment. Mice were monitored daily for signs of illness and sacrificed upon reaching a tumor volume of 2000 mm³ or immediately if they met other pre-specified humane endpoints as described above. Mice receiving TP7-56 and TP7-38 MSLN CAR-T constructs showed a median survival of 132 and 87 days respectively compared to median survival of 77 days in mice left untreated or given mock T cells. Overall, TP7-56 showed the best rate of survival overall though both TP7-56 and TP7-38 CAR-T showed significant survival benefit over no treatment.

Similarly, to described above, an additional experiment was undertaken to the in vivo activity of EGFR and MSLN-CAR cells in combination. In brief, CAR-T products were generated as described above using a single blood donor as a source of primary T cells. Specifically, CAR-T were generated for MSLN-specific TP7-56 CAR, EGFR-specific CAR, or using a 1:1 combination of MSLN-TP7-56 and EGFR CAR-lentivirus to generate a mixed CAR-T product. Mice were then injected with H292 lung tumour cells subcutaneously, followed by CAR-T treatment with a marginal dose of 1 million CAR-T cells. As showing in FIG. 19, while at this dose both MSLN-TP7-56 and EGFR had only partial therapeutic effects, a mixed CAR-T product containing both MSLN- and EGFR-specific CAR resulted in dramatically reduced tumour loads. These results demonstrate that generation of mixed CAR-T products using MSLN-CARs can lead to enhanced therapeutic effect.

### Discussion

NSG mice are widely used to study the interactions between the human immune system and cancer, a practical platform for evaluating immunotherapeutics in the context of human immune cells and human tumors. Overall, these results clearly demonstrate anti-cancer activity of MSLN-targeting single domain CAR modified T cells in vivo, similar to in vitro, and demonstrate therapeutic potential of these antibodies as tumor targeting moieties within CAR-T cells. Their ability to effectively and specifically target cells expressing MSLN antigen also provides evidence for their therapeutic potential beyond CAR-T therapy. Furthermore, combinatorial use of MSLN-CAR with other antigen-targeted CARs can result in enhanced therapeutic effect.

### Example 5: T-cell Engager Constructs

### Introduction

Similar to chimeric antigen receptor technology, novel antigen binding elements can also be linked to CD3-engaging antibody elements in order generate a soluble molecule that can simultaneously bind T cells and cellular target molecules, resulting in an antigen-specific T cell activation signal. This type of molecule, referred to as a bi-specific T cell engagers (BITE), is exemplified by Blinatumomab, wherein a single molecule simultaneously engages human CD19 and human CD3; used as a therapy for CD19 expressing B-cell family malignancies. In order to assess whether the human MSLN-specific single domain antibodies generated herein could be used in such a bi-specific T cell engager molecule, molecules were generated wherein one end of the molecule was comprised of a MSLN-specific single domain antibody sequence and the other end was comprised of a CD3-engager molecule. These novel bi-specific T cell engagers were then screened for non-specific and antigen-specific induction of T cell activation and T cell killing of target cells.

### Materials and Methods

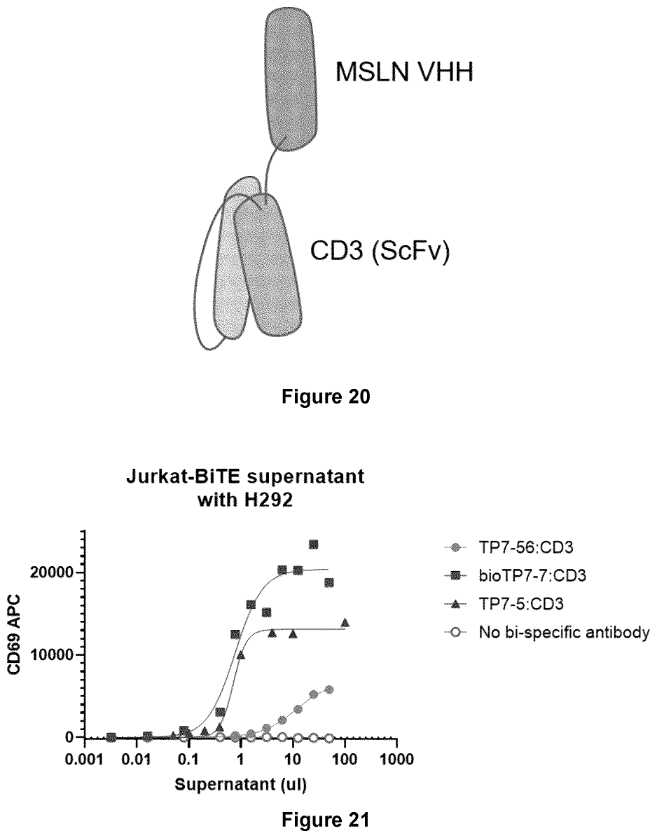

Single domain antibody antigen binding sequences were transferred to a modular bi-specific T cell engager DNA sequence [SEQ. ID 66] within a plasmid backbone; the DNA sequence used contains restriction sites to allow efficient recombination wherein the antigen binding domain could be replaced with the novel MSLN-antigen binding domain (ABD) sequences. Specific bi-specific T cell engager design used was as follows: Human CD28 signal peptide (SEQ ID NO: 52), sdAb antibody (ABD) (e.g., any one of SEQ ID NOs: 34 to 44), flexible linker domain (SEQ ID NO: 53), human CD8 hinge domain (SEQ ID NO: 54), short flexible linker domain (SEQ ID NO: 59), and a CD3-specific single chain variable fragment sequence. A model of MSLN-CD3 bi-specific T cell engager molecules with or without the inclusion of a hinge/spacer domain is provided (FIG. 20). Constructs were generated using golden gate assembly and confirmed using Sanger sequencing before proceeding to downstream testing.

To generate purified protein forms of bi-specific T cell engager molecules, plasmid DNA containing various constructs were transfected into HEK293T cells using polyethylenimine via standard process. Transfected cells were placed in cell culture and supernatant was collected over several days. Supernatant from MSLN-CD3 bispecific antibody TP7-5 with or without a hinge element between the 2 targeting moieties or a control BCMA-CD3 bi-specific antibody were then tested for bi-specific T cell engager activity by placing supernatant directly on Jurkat cells alone or in co-culture with MSLN-positive (H292) or MSLN-negative (Raji) target cells and incubated under standard conditions overnight. Jurkat cells were then examined for T cell activation using antibody staining for the human CD69 marker and flow cytometric analysis (FIGS. 21A and 21B). Results demonstrate that when delivered in solution, a MSLN-sdAb targeted bi-specific T cell engager without a hinge element can induce strong target dependent T cell activation. This activity was abrogated with the introduction of a hinge element extending the spacer domain between the 2 engager moieties. This may be reflective of the specific binding epitope of the TP7-5 single domain antibody. Epitope mapping for select binders identified as hit candidates are ongoing. Nevertheless, as demonstrated for CAR-T by McComb et al. (2022), these results points to the possibility of fine tuning the activity of BiTE constructs by modulating the hinge length.

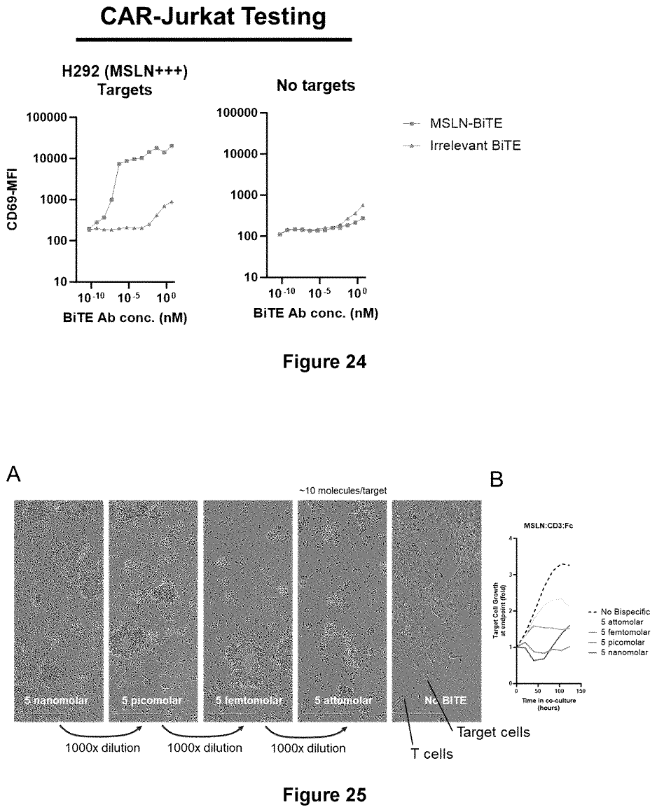

One challenge with bispecific engager constructs (e.g. BiTE and BIKE) is in production and purification of such molecules, due to the instability and tendency of single-chain variable fragments for oligomerization. A strategy to improve solubility, stability, and make purification easier for such molecules is conjugation to a human Fc domain. Thus, we generated constructs wherein the MSLN-VHH was linked to a single-chain variable fragment CD3 element, followed by a human Fc domain. This construct then spontaneously dimerizes in solution, resulting in a dimeric CD3-engager as depicted in FIG. 23. Functional testing with this dimeric MSLN-targeted T-cell engager molecule using unmodified Jurkat T cells and MSLN-high H292 cells as described above demonstrated detectable T-cell activation at doses of 10⁻⁹ nM (1 attomole) as shown in FIG. 24.

Functional testing of dimeric MSLN-VHH CD3-engager linked to human Fc was performed by combining primary human T cells with MSLN-high H292 lung cancer cells. Co-cultures demonstrated evidence of increased T-cell killing of H292 cells at dimeric MSLN-targeted bispecific T-cell engager doses in the attomolar range, as shown in FIG. 25.

### Results

FIG. 20 depicts the molecular structure of MSLN-specific single domain antibody bi-specific T cell engager proteins without the inclusion of an additional hinge/spacer domain; with a MSLN-sdAb sequence at the 5′ end of a DNA construct, followed by a linker sequence which can be of varying composition, followed by a CD3-specific single chain variable fragment.

FIG. 21 depict the results of Jurkat cell bi-specific T cell engager activation activity assay wherein HEK293T supernatants containing MSLN or control supernatant were placed on top of co-cultures containing Jurkat cells and MSLN-positive (H292) target cells. Graphs depict the average CD69-specific antibody staining of Jurkat cells as measured by flow cytometry. Results demonstrate MSLN-antigen specific activation of T cells in the presence of novel MSLN-sdAb bi-specific T cell engager molecules.

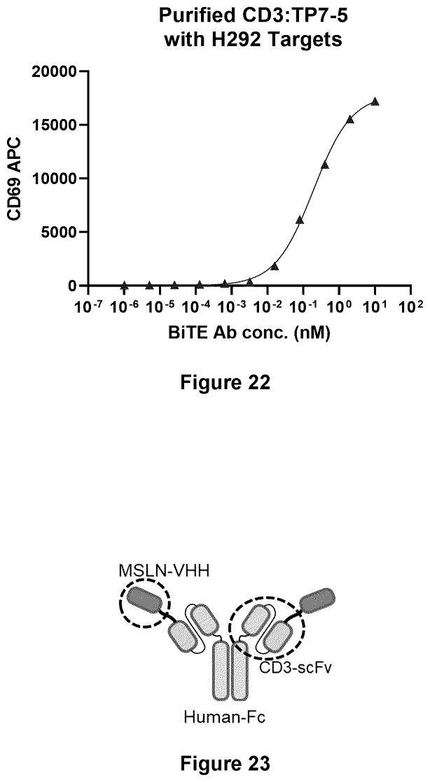

FIG. 22 depicts the results of a Jurkat bi-specific T cell engager activation activity assay wherein a bispecific antibody molecule was produced using E. coli bacteria and purified using affinity column. The purified bispecific molecule was then placed at varying doses based on the protein concentration in a co-culture assay containing Jurkat and MSLN+ target cells (H292). Graphs depict the average CD69-specific antibody staining of Jurkat cells as measured by flow cytometry. Error bars present the standard error of the mean over 2 duplicate co-culture wells. Results demonstrate an EC50 of approximately 1 nM for a purified TP7-5-CD3 bispecific T cell engager molecule.

FIG. 23 depicts the molecular structure of dimeric MSLN-specific single domain antibody bi-specific T cell engager proteins a MSLN-sdAb domain, followed by a linker sequence which can be of varying composition, followed by a CD3-specific single chain variable fragment, followed by a linker sequence, followed by a human Fc domain. This molecule will spontaneously dimerize in culture due to the presence of a human Fc domain, resulting in the dimeric T-cell engager as shown.

FIG. 24 depicts the results of a Jurkat bi-specific T cell engager activation activity assay wherein a dimeric MSLN-targeted bispecific antibody molecule was produced and purified using protein A purification. The purified molecule was then combined at varying doses with Jurkat T cells in co-culture with MSLN-high H292 lung cancer cells. Graphs depict the average CD69-specific antibody staining of Jurkat cells as measured by flow cytometry. Data demonstrated T-cell activating activity at extremely low doses with the dimeric engager molecule.

FIG. 25 depicts the results of a primary T cell functional testing assay for a dimeric bi-specific T cell engager molecule. A MSLN-VHH-CD3scFv-human Fc molecule was produced and purified as described above, before being combined at varying doses with primary human T cells in co-culture with MSLN-high H292 lung cancer cells. Images show T-cell mediated killing of larger H292 cancer cells with dimeric MSLN T-cell engager doses between 5 nanomolar and 5 attomolar concentration.

### Discussion

Overall, these results exemplify that MSLN-specific single domain binders can generate strong antigen-driven T cell activation signaling when combined in a bi-specific T cell engager molecule. MSLN-sdAb targeted bi-specific T cell engager molecules are demonstrated to drive target specific T cell activation and direct target cell killing by primary human T cells. While exemplary data is provided for a single MSLN-specific single domain antibodies, this data indicates that additional high affinity MSLN-binders described in this application are likely to have similar activity. Furthermore, molecular optimization may be performed in order to further increase functionality of bi-specific T cell engager molecules. In addition, combining multiple MSLN-specific single domain antibody sequences in a single molecule may be an effective strategy to increase target-specific activating activity. Addition of a human Fc-domain can increase construct stability and ease purification, resulting in a dimeric MSLN-targeted T-cell engager molecule with extremely high potency, resulting in detectable MSLN-specific activation of T cells at attomolar doses.

### Example 6: MSLN-Specific NK Cell Engager Constructs

### Introduction

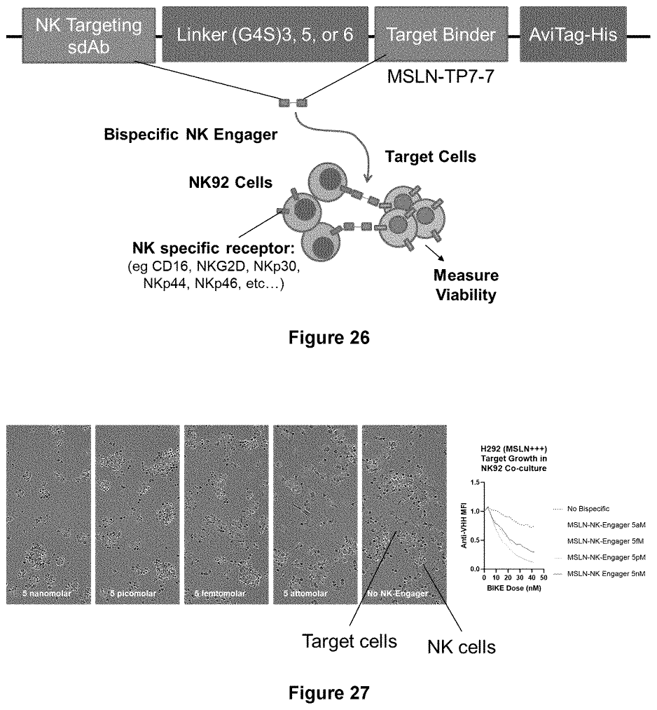

Similarly, to T-cell engaging bispecific antibodies, it is also possible to generate bispecific antibody molecules that can simultaneously engage NK cells and target cells to induce a target-specific cytotoxic reaction. Similarly, as above an NK-specific antibody moiety is linked to a MSLN-VHH sdAb antibody (ABD) (e.g., any one of SEQ ID NOs: 34 to 44) via a flexible linker domain (SEQ ID NO: 53), as shown in FIG. 26. The NK-specific element in this case could be specific for CD16, NKG2D, NKp44, NKp30, NKp46, or other NK-specific receptor. Constructs were generated using golden gate assembly and confirmed using Sanger sequencing before proceeding to downstream testing.

### Materials and Methods

To generate purified protein forms of bi-specific NK cell engager molecules, plasmid DNA containing various constructs were generated and protein was produced from E coli bacteria. Protein A columns were then used to purify the bispecific NK engager proteins. Functional testing was then performed by combining immortalized NK92 cells with MSLN-expressing H292 target cells as shown in the bottom part of FIG. 20. Results demonstrate that when delivered in solution, a MSLN-sdAb targeted bi-specific NK cell engager can induce more rapid and complete NK-mediated killing of target cells (FIG. 27).

### Results

FIG. 27 depicts the results of an NK92 co-culture functional testing assay for a bi-specific NK cell engager molecule. An NK-engager-MSLN-VHH molecule was produced and purified using E coli bacteria and protein A columns. The purified bispecific Nk engager was then combined at varying doses with NK92 cells in co-culture with MSLN-high H292 lung cancer cells. Images show accelerated NK-cell mediated killing of larger H292 cancer cells with MSLN NK-cell engager doses between 5 nanomolar and 5 attomolar concentration.

### Discussion

Overall, these results exemplify that MSLN-specific single domain binders can generate strong antigen-driven NK cell activation signaling when combined in a bi-specific NK cell engager molecule. An NK-engaging bispecific MSLN-targeted NK cell engager molecule shows high potency, resulting increases in clear MSLN-specific activation of NK cells at femtomolar doses.

### Example 7: Evaluating Target Specificity of Anti-MSLN VHHs by immunohistochemistry

### Introduction

MSLN, although is overexpressed in a wide range of human cancers, is also expressed in select set of normal tissues. Thus, on-target, off tumor toxicity is a potential safety concern when developing therapeutics targeting MSLN. Furthermore, a potential issue that any novel binder can encounter is cross-reactivity to other antigens which could lead to undesirable side effects. In order to assess whether the MSLN single domain antibodies has any cross-reactivity to tissue antigens expressed in normal human tissues, the binding specificity of a select set of MSLN single domain antibodies that were chosen as lead CAR-T candidates were evaluated using healthy vs, tumor human tissue microarray (TMA) via immunohistochemistry (IHC).

### Material and Methods

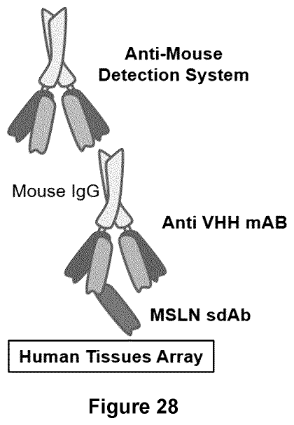

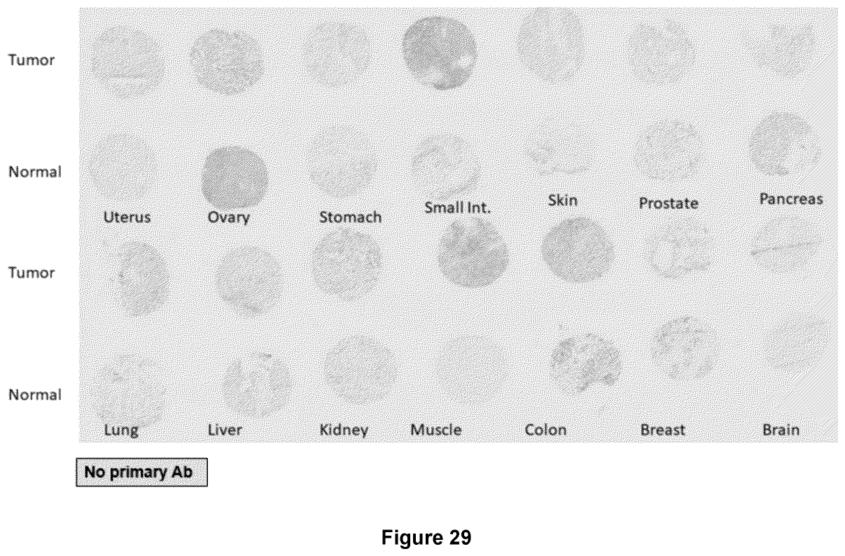

Select set of purified anti-hMSLN VHH (TP7-5, TP7-7 TP7-9, TP7-38, TP7-56) were used to assess the tissue binding specificity by immunohistochemistry. Frozen normal and tumor tissue arrays were purchased from BioChain (Catalogue number T6235700-5). Binding specificity was assessed using purified MSLN VHH or an irrelevant control VHH as primary antibody. An anti-llama VHH monoclonal antibody was used as the secondary antibody with a HRP labelled anti-mouse IgG antibody as the detection antibody. A qualified standard operating procedure was used for the TMA staining. The IHC assay schema is depicted in FIG. 28. Stained TMAs results are presented in FIGS. 29-32.

### Results

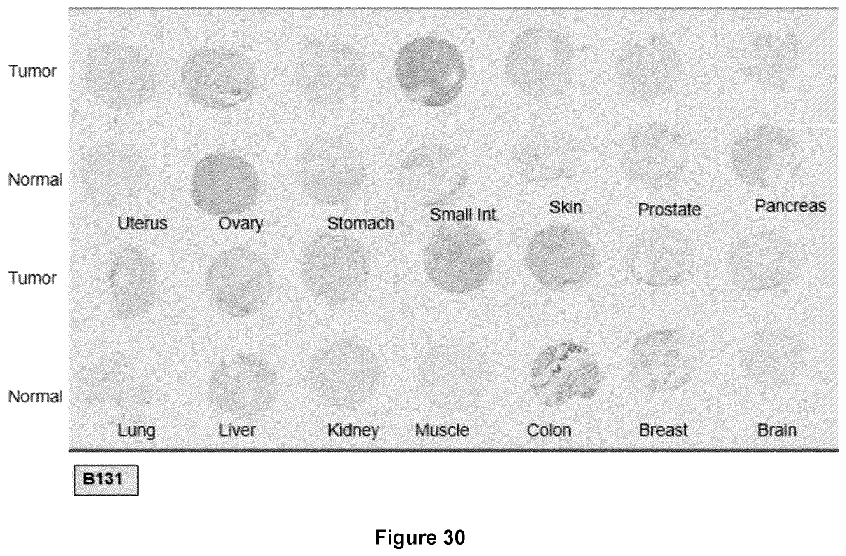

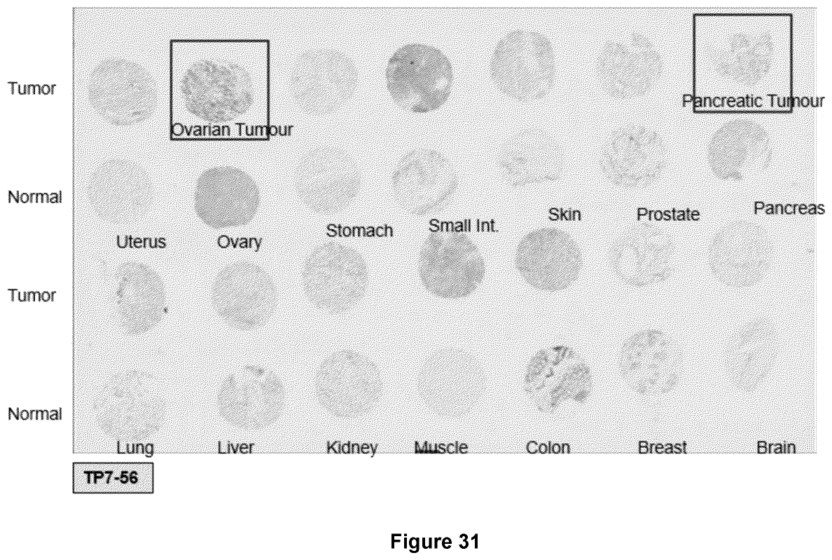

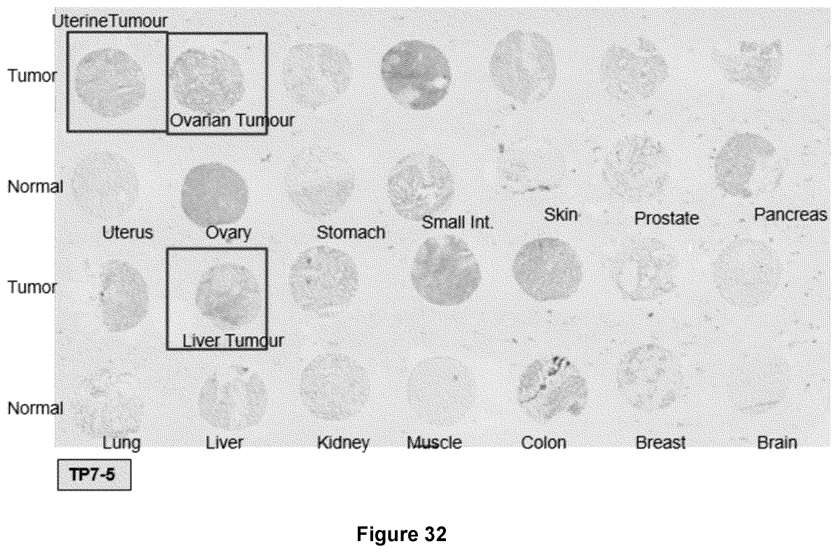

FIG. 28 depicts the IHC assay layout. FIGS. 24, 25, 26 and 27 depict low resolution images of the stained tissue arrays where either no primary antibody was used (FIG. 29), or irrelevant VHH targeting Clostridium difficile toxin B (B131) (FIG. 30), MSLN VHH TP7-56 (FIG. 31), or MSLN VHH TP7-5 (FIG. 32) respectively were used as primary antibody respectively. Low level of background staining was observed in some tissues even in the absence of any primary antibodies. Similar or slightly higher level of background staining was seen with the same tissues when the irrelevant VHH B131 was used. However, clear staining was seen with ovarian, pancreatic tumor but not normal healthy tissues when TP7-56 was used as primary antibody. Clear staining was also observed with TP7-5 in ovarian, uterine and liver tumor tissues but not the healthy tissues from same organs.

### Discussion

Overall, the IHC of human normal vs. tumor tissue arrays demonstrated positive staining of a number of tumor tissue samples by MSLN VHH which was absent in normal tissues. The tumor tissues showing positive staining are tissues types that are commonly reported to over express MLSN. Such differential staining was absent with the irrelevant B131 VHH. The low level of non-specific background staining observed in multiple tissues with B131 or no primary antibody was likely resulting from the anti-VHH secondary antibody. This background staining did not interfere with the assessment of specific binding of primary antibody. Overall, there were no concerning off-target binding seen with the single domain MSLN binders tested.

### General Discussion of Examples

This is a demonstration of novel single domain antibodies for application in MSLN targeted immunotherapies with specific data driven evidence for their application in CAR-T and bi-specific T cell engager treatment modalities. Single domain antibodies offer significant advantage over the single-chain variable fragment antibodies which are typically used in the antigen recognition domain of CAR constructs, including significantly smaller size, higher homology with human antibody sequences, enhanced modularity, and ability to target epitopes which may not be accessible to scFvs. The single domain antibodies may be combined with other single domain antibodies targeting antigen that are co-expressed with MSLN to generate therapeutic construct targeting human cancers.

In the preceding description, for purposes of explanation, numerous details are set forth in order to provide a thorough understanding of the embodiments. However, it will be apparent to one skilled in the art that these specific details may not be required.

The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

### REFERENCES

1. Bera, T. K. & Pastan, I. Mesothelin Is Not Required for Normal Mouse Development Reproduction. Mol. Cell. Biol. 20, 2902-2906 (2000).
2. Tang, Z., Qian, M. & Ho, M. The role of mesothelin in tumor progression and targeted therapy. Anticancer Agents Med. Chem. 13, 276-280 (2013).
3. Cheng, W.-F. et al. High mesothelin correlates with chemoresistance and poor survival in epithelial ovarian carcinoma. Br. J. Cancer 100, 1144-1153 (2009).
4. Li, M. et al. Mesothelin is a malignant factor and therapeutic vaccine target for pancreatic cancer. Mol. Cancer Ther. 7, 286-296 (2008).
5. Hassan, R. et al. Mesothelin Immunotherapy for Cancer: Ready for Prime Time? J. Clin. Oncol. 34, 4171-4179 (2016).
6. An Efficacy Study of MORAb-009 in Subjects with Pancreatic Cancer-Study Results ClinicalTrials.gov. Available at: https://clinicaltrials.gov/ct2/show/results/NCT00570713.
7. Hassan, R. et al. Major Cancer Regressions in Mesothelioma after Treatment with an Anti-Mesothelin Immunotoxin and Immune Suppression. Sci. Transl. Med. 5, 208ra147 (2013).
8. Beatty, G. L. et al. Mesothelin-specific CAR mRNA-Engineered T cells Induce Anti-Tumor Activity in Solid Malignancies. Cancer Immunol. Res. 2, 112-120 (2014).
9. Beatty, G. L. et al. Safety and antitumor activity of chimeric antigen receptor modified T cells in patients with chemotherapy refractory metastatic pancreatic cancer. J. Clin. Oncol. 33, 3007-3007 (2015).
10. O'Hara, M., Stashwick, C., Haas, A. R. & Tanyi, J. L. Mesothelin as a target for chimeric antigen receptor-modified T cells as anticancer therapy. Immunotherapy 8, 449-460 (2016).
11. Hatterer E, Chauchet X, Richard F, et al. Targeting a membrane-proximal epitope on mesothelin increases the tumoricidal activity of a bispecific antibody blocking CD47 on mesothelin-positive tumors. MAbs. 12 (1): 1739408 (2020).
12. Faust, J. R.; Hamill, D.; Kolb, E. A.; Gopalakrishnapillai, A.; Barwe, S. P. Mesothelin: An Immunotherapeutic Target beyond Solid Tumors. Cancers 2022, 14, 1550. https://doi.org/10.3390/cancers1406155
13. Bloemberg, D. et al. A High-Throughput Method for Characterizing Novel Chimeric Antigen Receptors in Jurkat Cells. Cell 16, 238-254 (2020).
14. McComb, S. et al. Programmable Attenuation of Antigenic Sensitivity for a Nanobody-Based EGFR Chimeric Antigen Receptor Through Hinge Domain Truncation. Front. Immunol. 13:864868 (2022).
15. US 2018/0002439 A1 to Baty et al (Jan. 4, 2018), “Anti-Mesothelin Antibodies and Uses thereof”.
16. US 2017/0267755 A1 to Scholler (Sep. 21, 2017), “Isolated Anti-Mesothelin Antibodies, Conjugates and Uses thereof”

All references referred to herein are expressly incorporated by reference.

TABLE 4
Table of Sequences

| Seq. # | Description | | Sequence |
|---|---|---|---|
| 1 | hMSLN-TP7-4 | CDR1 | GLTFGSYA |
| 2 | hMSLN-TP7-4 | CDR2 | ISSRGVT |
| 3 | hMSLN-TP7-4 | CDR3 | NAKGPFMLREYDY |
| 4 | hMSLN-TP7-5 | CDR1 | GRSISDYF |
| 5 | hMSLN-TP7-5 | CDR2 | ISWSGGTA |
| 6 | hMSLN-TP7-5 | CDR3 | AARLSYDSRTSLQNSYDYNY |
| 7 | hMSLN-TP7-9 | CDR1 | GRTVSTYN |
| 8 | hMSLN-TP7-9 | CDR2 | INWTGGST |
| 9 | hMSLN-TP7-9 | CDR3 | AATRASWTENTY |
| 10 | hMSLN-TP7-18 | CDR1 | GRAFKWWT |
| 11 | hMSLN-TP7-18 | CDR2 | ISWSDGST |
| 12 | hMSLN-TP7-18 | CDR3 | ARYGKDLSGRHSKNVQDYDY |
| 13 | hMSLN-TP7-35 | CDR1 | ASTERFFA |
| 14 | hMSLN-TP7-35 | CDR2 | ISRDGDA |
| 15 | hMSLN-TP7-35 | CDR3 | HTRILSDHY |
| 16 | hMSLN-TP7-38A | CDR1 | GRTVSAYN |
| 17 | hMSLN-TP7-38A | CDR2 | INWTGGGT |
| 18 | hMSLN-TP7-38A | CDR3 | AATRTSWTENTY |
| 19 | hMSLN-TP7-38B | CDR1 | GRTVSTYN |
| 20 | hMSLN-TP7-38B | CDR2 | INWTGGGT |
| 21 | hMSLN-TP7-38B | CDR3 | AATRTSWTENTY |
| 22 | hMSLN-TP7-56 | CDR1 | GRDFSSHF |
| 23 | hMSLN-TP7-56 | CDR2 | ISWSGGTT |
| 24 | hMSLN-TP7-56 | CDR3 | AARRTYDSRTSLRNSYDYPY |
| 25 | hMSLN-bioTP7-7 | CDR1 | GRTVSTYN |
| 26 | hMSLN-bioTP7-7 | CDR2 | INWTGGST |
| 27 | hMSLN-bioTP7-7 | CDR3 | AATRTSWTENTY |
| 28 | hMSLN-biomTP7-41 | CDR1 | GIVRASG |
| 29 | hMSLN-biomTP7-41 | CDR2 | ITTGGETT |
| 30 | hMSLN-biomTP7-41 | CDR3 | NQRSY |
| 31 | hMSLN-bioTP7-48 | CDR1 | GSIWSASG |
| 32 | hMSLN-bioTP7-48 | CDR2 | ITTGGETT |
| 33 | hMSLN-bioTP7-48 | CDR3 | NSRSY |
| 34 | hMSLN-bioTP7-53 | CDR1 | GGIGSLSN |
| 35 | hMSLN-bioTP7-53 | CDR2 | ITGQSTT |
| 36 | hMSLN-bioTP7-53 | CDR3 | NVATLRNEY |
| 37 | hMSLN-TP7-75A | CDR1 | GRTFSASA |
| 38 | hMSLN-TP7-75A | CDR2 | VAWGGRDT |
| 39 | hMSLN-TP7-75A | CDR3 | AAGQRGWSHSTSRDYDY |
| 40 | hMSLN-TP7-75B | CDR1 | GRTFSASA |
| 41 | hMSLN-TP7-75B | CDR2 | VAWGGRDT |
| 42 | hMSLN-TP7-75B | CDR3 | AAGQRGWSHSTSSDYDY |
| 43 | hMSLN-mTP7-82 | CDR1 | GSIRSASG |
| 44 | hMSLN-mTP7-82 | CDR2 | ITTSGETT |
| 45 | hMSLN-mTP7-82 | CDR3 | NLRSY |
| 46 | hMSLN-TP7-4 | Full | QVQLVGSGGGLVRAGGSLRLSCAGSGLTFGSYAMAWYRQAPGK |
| | | length | QRELVASISSRGVTNYADSVEGRFTISRDNAKSTVYLQMNDLK |
| | | | PEDAAVYYCNAKGPFMLREYDYWGQGTQVTVSS |
| 47 | hMSLN-TP7-5 | Full | QVKLEESGGGLVQAGGSLRISCAASGRSISDYFMGWFRQTPGK |
| | | length | EREFVAAISWSGGTAYYADSVKGRFTISRDNAKNTGYLQMNSL |
| | | | KPEDTAVYFCAARLSYDSRTSLQNSYDYNYWGQGTQVTVSS |
| 48 | hMSLN-TP7-9 | Ful | DVQLVDSGGGLVQAGGSLRLSCAASGRTVSTYNMAWFRQAPGK |
| | | length | EREIVAVINWTGGSTYYAESVKGRFTISGDNPKNTVYLQMNSL |
| | | | KPEDTAVYYCAATRASWTENTYWGQGTQVTVSS |
| 49 | hMSLN-TP7-18 | Full | EVQLVESGGRLVQAGGSLRLSCVASGRAFKWWTMGWFRQAPGK |
| | | length | EREFVASISWSDGSTTYGDSVKGRFAISRDNNKNTVYLQMNSL |
| | | | TPEDTAVYYCARYGKDLSGRHSKNVQDYDYWGQGTQVTVSS |
| 50 | hMSLN-TP7-35 | Full | QVQLVDSGGGLVQPGGSLRLSCTTSASTFRFFAMHWYRQAPGK |
| | | length | ERELVAVISRDGDAEYVDSVKGRFTISRDNAKNAVYLQMNSLK |
| | | | PEDTAVYYCHTRILSDHYWGQGTQVTVSS |
| 51 | hMSLN-TP7-38A | Full | QVKLEESGGGLVQPGGSLRLSCAASGRTVSAYNMAWFRQAPGK |
| | | length | EREIVAVINWTGGGTYYAESVKGRFTISRDNPKNTVYLQMNSL |
| | | | KPEDTAVYYCAATRTSWTENTYWGQGTQVTVSS |
| 52 | hMSLN-TP7-38B | Full | QVQLVESGGGLVQPGGSLRLSCAASGRTVSTYNMAWFRQAPGK |
| | | length | EREIVAVINWTGGGTYYAESVKGRFTISRDNPKNTVYLQMNSL |
| | | | KPEDTAVYYCAATRTSWTENTYWGQGTQVTVSS |
| 53 | hMSLN-TP7-56 | Full | QVQLVESGGGLVQAGGSLRISCVASGRDFSSHFMGWSRQAPGK |
| | | length | EREFVAAISWSGGTTYYADSVKGRFAISRDNDKTTVYLQMNSL |
| | | | KPEDSAVYYCAARRTYDSRTSLRNSYDYPYWGQGTQVTVSS |
| 54 | hMSLN-bioTP7-7 | Full | QVQLVESGGGLVQAGGSLRLSCAASGRTVSTYNMAWFRQAPGK |
| | | length | EREIVAVINWTGGSTYYAESVKGRFTISRDNPKNTVYLQMNSL |
| | | | KPEDTAVYYCAATRTSWTENTYWGQGTQVTVSS |
| 55 | hMSLN-biomTP7- | Full | QVQLVESGGGLVQPGGSLRLSCTASGIVRASGMAWYRQAPGKQ |
| | 41 | length | RELVASITTGGETTWYADSVKGRFTISIDNAKMMLYLQMNALK |
| | | | PEDTAVYLCNQRSYWGQGTQVTVSS |
| 56 | hMSLN-bioTP7-48 | Full | QVKLEESGGGLVQPGGSLRLSCTASGSIWSASGLTWYRQRPGN |
| | | length | ERELVASITTGGETTWYADSVKGRFTISISRDNGKMITYLQMN |
| | | | SLKPEDTAVYLCNSRSYWGQGTQVTVSS |
| 57 | hMSLN-bioTP7-53 | Full | QVQLVESGGGLVQPGGSLRLACAASGGIGSLSNVRWYRQGPGK |
| | | length | EREYVAGITGQSTTYYADSVKGRFTISRDNVKNTVDLQMNSLK |
| | | | PEDTAVYICNVATLRNEYWGQGTQVTVSS |
| 58 | hMSLN-TP7-75A | Full | QVQLVESGGGLVQAGGSLSLSCAASGRTFSASAMAWFRQAPGT |
| | | length | DREFVAAVAWGGRDTHYADSVKGRFTISMNNAKNTVVLQMNSL |
| | | | KPEDTAVYYCAAGQRGWSHSTSRDYDYWGQGTQVTVSS |
| 59 | hMSLN-TP7-75B | Full | QVQLVESGGGLVQAGGSLSLSCAASGRTFSASAMAWFRQAPGT |
| | | length | DREFVAAVAWGGRDTHYADSVKGRFTISMNNAKNTVVLQMNSL |
| | | | KPEDTAVYYCAAGQRGWSHSTSSDYDYWGQGTQVTVSS |
| 60 | hMSLN-mTP7-82 | Full | QVQLVESGGGLVQAGGSLRLSCAASGSIRSASGMHWYRQAPGK |
| | | length | ERELVASITTSGETTWYRDSVKGRFTISINNAKMMLYLQMNSL |
| | | | KFEDTAVYYCNLRSYWGQGTQVTVSS |
| 61 | MSLN-CAR | | MLRLLLALNLFPSIQVTGQVQLVESGGGLVQPGGSLRLSCAAS |
| | TP7-38-CD28TM-BBZ | | GRTDSTYNMAWFRQAPGKEREIVAVINWTGGGTYYAESVKGRF |
| | | | TISRDNPKNTVYLQMNSLKPEDTAVYYCAATRTSWTENTYWGQ |
| | | | GTQVTVSSPLTTTPAPRPPTPAPTIASQPLSLRPEACRPAAGG |
| | | | AVHTRGLDFACDIYIWAPLAGTCGVLLLSLVITLYCKRGRKKL |
| | | | LYIFKQPFMRPVQTTQEEDGCSCRFPEEEEGGCELLRVKESRS |
| | | | ADAPAYQQGQNQLYNELNLGRREEYDVLDKRRGRDPEMGGKPQ |
| | | | RRKNPQEGLYNELQKDKMAEAYSEIGMKGERRRGKGHDGLYQG |
| | | | LSTATKDTYDALHMQALPPR |

| | | |
|---|---|---|
| 62 | MSLN-CAR | MLRLLLALNLFPSIQVTGQVQLVESGGGLVQPGGSLRLSCAAS |
| | TP7-38-CD8TM-BBz | GRTDSTYNMAWFRQAPGKEREIVAVINWTGGGTYYAESVKGRF |
| | | TISRDNPKNTVYLQMNSLKPEDTAVYYCAATRTSWTENTYWGQ |
| | | GTQVTVSSPLRDDGGGGSGGGGSGGGGSGGTTTPAPRPPTPAP |
| | | TIASQPLSLRPEACRPAAGGAVHTRGLDFACDVVSPSKPFWVL |
| | | VVVGGVLACYSLLVTVAFIIFWVRKRGRKKLLYIFKQPEMRPV |
| | | QTTQEEDGCSCRFPEEEEGGCELLRVKFSRSADAPAYQQGQNQ |
| | | LYNELNLGRREEYDVLDKRRGRDPEMGGKPQRRKNPQEGLYNE |
| | | LQKDKMAEAYSEIGMKGERRRGKGHDGLYQGLSTATKDTYDAL |
| | | HMQALPPR |
| 63 | MSLN-CAR | MLRLLLALNLFPSIQVTGQVQLVESGGGLVQAGGSLRISCVAS |
| | TP7-56-CD28TM-BBz | GRDFSSHFMGWSRQAPGKEREFVAAISWSGGTTYYADSVKGRF |
| | | AISRDNDKTTVYLQMNSLKPEDSAVYYCAARRTYDSRTSLRNS |
| | | YDYPYWGQGTQVTVSSPLRDDGGGGSGGGGSGGGGSGGTTTPA |
| | | PRPPTPAPTIASQPLSLRPEACRPAAGGAVHTRGLDFACDVVS |
| | | PSKPFWVLVVVGGVLACYSLLVTVAFIIFWVRKRGRKKLLYIF |
| | | KQPFMRPVQTTQEEDGCSCRFPEEEEGGCELLRVKFSRSADAP |
| | | AYQQGQNQLYNELNLGRREEYDVLDKRRGRDPEMGGKPQRRKN |
| | | PQEGLYNELQKDKMAEAYSEIGMKGERRRGKGHDGLYQGLSTA |
| | | TKDTYDALHMQALPPR |
| 64 | MSLN-CAR | MLRLLLALNLFPSIQVTGQVQLVESGGGLVQAGGSLRISCVAS |
| | TP7-56-CD8TM-BBZ | GRDFSSHFMGWSRQAPGKEREFVAAISWSGGTTYYADSVKGRF |
| | | AISRDNDKTTVYLQMNSLKPEDSAVYYCAARRTYDSRTSLRNS |
| | | YDYPYWGQGTQVTVSSPLTTTPAPRPPTPAPTIASQPLSLRPE |
| | | ACRPAAGGAVHTRGLDFACDIYIWAPLAGTCGVLLLSLVITLY |
| | | CKRGRKKLLYIFKQPFMRPVQTTQEEDGCSCRFPEEEEGGCEL |
| | | LRVKFSRSADAPAYQQGQNQLYNELNLGRREEYDVLDKRRGRD |
| | | PEMGGKPQRRKNPQEGLYNELQKDKMAEAYSEIGMKGERRRGK |
| | | GHDGLYQGLSTATKDTYDALHMQALPPR |
| 65 | MSLN-CAR | MLRLLLALNLFPSIQVTGQVQLVESGGGLVQAGGSLRLSCAAS |
| | bioTP7-7-CD28TM-BBz | GRTVSTYNMAWFRQAPGKEREIVAVINWTGGSTYYAESVKGRF |
| | | TISRDNPKNTVYLQMNSLKPEDTAVYYCAATRTSWTENTYWGQ |
| | | GTQVTVSSPLRDDGGGGSGGGGSGGGGSGGTTTPAPRPPTPAP |
| | | TIASQPLSLRPEACRPAAGGAVHTRGLDFACDVVSPSKPFWVL |
| | | VVVGGVLACYSLLVTVAFIIFWVRKRGRKKLLYIFKQPFMRPV |
| | | QTTQEEDGCSCRFPEEEEGGCELLRVKFSRSADAPAYQQGQNQ |
| | | LYNELNLGRREEYDVLDKRRGRDPEMGGKPQRRKNPQEGLYNE |
| | | LQKDKMAEAYSEIGMKGERRRGKGHDGLYQGLSTATKDTYDAL |
| | | HMQALPPR |
| 66 | MSLN-CAR | MLRLLLALNLFPSIQVTGQVQLVESGGGLVQAGGSLRLSCAAS |
| | bioTP7-7-CD8TM-BBZ | GRTVSTYNMAWFRQAPGKEREIVAVINWTGGSTYYAESVKGRF |
| | | TISRDNPKNTVYLQMNSLKPEDTAVYYCAATRTSWTENTYWGQ |
| | | GTQVTVSSPLTTTPAPRPPTPAPTIASQPLSLRPEACRPAAGG |
| | | AVHTRGLDFACDIYIWAPLAGTCGVLLLSLVITLYCKRGRKKL |
| | | LYIFKQPFMRPVQTTQEEDGCSCRFPEEEEGGCELLRVKESRS |
| | | ADAPAYQQGQNQLYNELNLGRREEYDVLDKRRGRDPEMGGKPQ |
| | | RRKNPQEGLYNELQKDKMAEAYSEIGMKGERRRGKGHDGLYQG |
| | | LSTATKDTYDALHMQALPPR |
| 67 | CAR modular construct | ATGCTCAGGCTGCTCTTGGCTCTCAACTTATTCCCTTCAATTC |
| | (“sdAb_cds” indicates the | AAGTAACAGGA[sdAb_cds]CCTTTGCGAGACGACGGTGGCG |
| | coding | GGGGATCAGGTGGTGGAGGTAGCGGGGGAGGGGGCTCAGGCGG |
| | sequence for any one of the | TACAACTACGCCTGCACCTCGCCCACCGACCCCAGCACCAACC |
| | sdAbs as described herein) | ATCGCTTCACAGCCTTTGAGCCTGCGACCAGAGGCATGTCGCC |
| | | CTGCTGCGGGCGGTGCCGTTCATACTCGCGGACTTGATTTTGC |
| | | GTGTGACGTCGTCTCGCCTTCTAAGCCCTTTTGGGTGCTGGTG |
| | | GTGGTTGGTGGAGTCCTGGCTTGCTATAGCTTGCTAGTAACAG |
| | | TGGCCTTTATTATTTTCTGGGTGAGGAAACGGGGCAGAAAGAA |
| | | ACTCCTGTATATATTCAAACAACCATTTATGCGACCAGTACAA |
| | | ACTACTCAAGAGGAAGATGGCTGTAGCTGCCGATTTCCAGAAG |
| | | AAGAAGAAGGAGGATGTGAACTGCTGAGAGTGAAGTTCAGCAG |
| | | GAGCGCAGACGCCCCCGCGTACCAGCAGGGCCAGAACCAGCTC |
| | | TATAACGAGCTCAATCTAGGACGAAGAGAGGAGTACGATGTTT |
| | | TGGACAAGCGACGTGGCCGGGACCCTGAGATGGGGGGAAAGCC |
| | | GCAGAGAAGGAAGAACCCTCAGGAAGGCCTGTACAATGAACTG |
| | | CAGAAAGATAAGATGGCGGAGGCCTACAGTGAGATTGGGATGA |
| | | AAGGCGAGCGCCGGAGGGGCAAGGGGCACGATGGCCTTTACCA |
| | | GGGACTCAGTACAGCCACCAAGGACACCTACGACGCCCTTCAC |
| | | ATGCAGGCCCTGCCCCCTCGC |
| 68 | Human CD28 Signal Peptide | MLRLLLALNLFPSIQVTG |
| 69 | Synthetic Flexible Linker | GGGGSGGGGSGGGGSGG |
| | Domain | |
| 70 | Human CD8 Hinge Domain | TTTPAPRPPTPAPTIASQPLSLRPEACRPAAGGAVHTRGLDFA |
| | | CD |
| 71 | Human CD8 Transmembrane | IYIWAPLAGTCGVLLLSLVITLYC |
| | Domain | |
| 72 | Human CD28 Transmembrane | PSKPFWVLVVVGGVLACYSLLVTVAFIIFWVR |
| | Domain | |
| 73 | Human 4-1BB Costimulatory | KRGRKKLLYIFKQPFMRPVQTTQEEDGCSCRFPEEEEGGCEL |
| | Domain | |
| 74 | Human CD3zeta Signaling | LRVKFSRSADAPAYQQGQNQLYNELNLGRREEYDVLDKRRGRD |
| | Domain | PEMGGKPQRRKNPQEGLYNELQKDKMAEAYSEIGMKGERRRGK |
| | | GHDGLYQGLSTATKDTYDALHMQALPPR |
| 75 | Short flexible linker amino | GGGGS |
| | acid sequence | |
| 76 | EGFR-MSLN-56-BBz Tandem- | MLRLLLALNLFPSIQVTGQVQLVESGGGLVQPGGSLRLSCSVS |
| | CAR amino acid sequence | LSDFGSKHLAWHRQAPGKEREWVALIFSSGATNYANSVKGRFT |
| | Exemplary sequence for a | ISRDNSKNTVYLQMNSLKPEDTAVYYCNLVPPSPAWGQGTQVT |
| | multi- | VSSPSGGGGQVQVQLVESGGGLVQAGGSLRISCVASGRDFSSH |
| | binder CAR construct | FMGWSRQAPGKEREFVAAISWSGGTTYYADSVKGRFAISRDND |
| | sdAb sequence is in bold | KTTVYLQMNSLKPEDSAVYYCAARRTYDSRTSLRNSYDYPYWG |
| | | QGTQVTVSSPSTTTPAPRPPTPAPTIASQPLSLRPEACRPAAG |
| | | GAVHTRGLDFACDPSKPFWVLVVVGGVLACYSLLVTVAFIIFW |
| | | VRKRGRKKLLYIFKQPFMRPVQTTQEEDGCSCRFPEEEEGGCE |
| | | LLRVKFSRSADAPAYQQGQNQLYNELNLGRREEYDVLDKRRGR |
| | | DPEMGGKPQRRKNPQEGLYNELQKDKMAEAYSEIGMKGERRRG |
| | | KGHDGLYQGLSTATKDTYDALHMQALPPR |
| 77 | Exemplary Bispecific T cell | ATGGAGTTTGGGCTGAGCTGGGTTTTCCTCGTTGCTCTTTTTA |
| | engager construct | GAGGTGTCCAGTGTACAGGAGGGTCTTCGCAGGTACAGCTGGT |
| | DNA sequence | GGAGTCTGGGGGAGGATTGGTGCAGGCTGGGGGCTCTCTGAGA |
| | bioTP7-7-CD3-1E2 | CTCTCCTGTGCAGCCTCTGGACGCACCGTCAGTACCTATAATA |
| | | TGGCCTGGTTCCGCCAGGCTCCAGGGAAGGAGCGTGAGATTGT |
| | | AGCAGTTATTAACTGGACTGGTGGTAGCACATACTATGCAGAG |
| | | TCCGTGAAGGGCCGATTCACCATCTCCAGAGATAACCCCAAGA |
| | | ACACGGTGTATTTACAAATGAACAGCCTGAAACCTGAGGACAC |
| | | GGCCGTTTATTACTGTGCAGCAACTAGGACATCCTGGACCGAG |
| | | AATACCTACTGGGGCCAGGGGACCCAGGTCACCGTTTCCTCAg |
| | | aagacttccttggaggaggcggaagtCAAGTGCAGTTGCAACA |
| | | ACCAGGTGCCGAGCTTGGGAAACCGGGTACCAGCGTTAAACTC |
| | | TCTTGCAAAGCATCCGGTTATACCTTTACTTCTTACTGGATCC |
| | | ATTGGGTCAAACAACGCCCAGGCCAGGGTCTCGAATGGGTGGG |
| | | CAACATCAACCCAAACAGCGGCTCTATCAATTATAACGAAAAG |
| | | TTTAAGAATAAAGCAACTCTCACGGTGGATAAGAGCAGCTCAA |
| | | CCGCTTATATGCAATTGAGTACTTTGACCAGTGAAGATTTCGC |
| | | TGTGTATTATTGCACGCGCGATACATCCGGACAGTATTATTTC |
| | | GATTATTGGGGCCAGGGGACAACCCTGACAGTGTCTAGCggcg |
| | | gtggtgggtcaggcggcggtgggagcggaggaggtggaagcGA |
| | | TATCGTAATGAGTCAATCCCCTAGCAGCTTGGCGGTAAGTGCT |
| | | GGGGAACGCGTTACAATGTCCTGTAAGAGCTCTCAATCTTTGC |
| | | TGAATTCCAGGACTAGGAAGAATTATCTGGCCTGGTATCAACA |
| | | AAAGCCCGGACAAAGTCCAAAGCTCCTGATTTATTGGGCTAGC |
| | | ACCAGAGAGTCAGGAGTACCAGACCGGTTTACTGGAAGCGGGT |
| | | CAGGAACCGACTTTACCCTGACAATATCCAGCGTCCAAGCAGA |
| | | AGATCTTGCCGTGTACTATTGTATTCAGTCCTACCACTTGCGC |
| | | ACTTTTGGGGGGGGAACAAAACTCGAGATAAAGCACCACCACC |
| | | ACCACCACTAG |
| 78 | Exemplary alternative Bi- | ATGGAGTTTGGGCTGAGCTGGGTTTTCCTCGTTGCTCTTTTTA |
| | specific | GAGGTGTCCAGTGTACAGGACAAGTGCAGTTGCAACAACCAGG |
| | T cell engager format | TGCCGAGCTTGGGAAACCGGGTACCAGCGTTAAACTCTCTTGC |
| | CD3-1E2-TP7-7 | AAAGCATCCGGTTATACCTTTACTTCTTACTGGATCCATTGGG |
| | | TCAAACAACGCCCAGGCCAGGGTCTCGAATGGGTGGGCAACAT |
| | | CAACCCAAACAGCGGCTCTATCAATTATAACGAAAAGTTTAAG |
| | | AATAAAGCAACTCTCACGGTGGATAAGAGCAGCTCAACCGCTT |
| | | ATATGCAATTGAGTACTTTGACCAGTGAAGATTTCGCTGTGTA |
| | | TTATTGCACGCGCGATACATCCGGACAGTATTATTTCGATTAT |
| | | TGGGGCCAGGGGACAACCCTGACAGTGTCTAGCggcggtggtg |
| | | ggtcaggcggcggtgggagcggaggaggtggaagcGATATCGT |
| | | AATGAGTCAATCCCCTAGCAGCTTGGCGGTAAGTGCTGGGGAA |
| | | CGCGTTACAATGTCCTGTAAGAGCTCTCAATCTTTGCTGAATT |
| | | CCAGGACTAGGAAGAATTATCTGGCCTGGTATCAACAAAAGCC |
| | | CGGACAAAGTCCAAAGCTCCTGATTTATTGGGCTAGCACCAGA |
| | | GAGTCAGGAGTACCAGACCGGTTTACTGGAAGCGGGTCAGGAA |
| | | CCGACTTTACCCTGACAATATCCAGCGTCCAAGCAGAAGATCT |
| | | TGCCGTGTACTATTGTATTCAGTCCTACCACTTGCGCACTTTT |
| | | GGGGGGGGAACAAAACTCGAGATAAAGggccttggaggaggcg |
| | | gaagtCAGGTACAGCTGGTGGAGTCTGGGGGAGGATTGGTGCA |
| | | GGCTGGGGGCTCTCTGAGACTCTCCTGTGCAGCCTCTGGACGC |
| | | ACCGTCAGTACCTATAATATGGCCTGGTTCCGCCAGGCTCCAG |
| | | GGAAGGAGCGTGAGATTGTAGCAGTTATTAACTGGACTGGTGG |
| | | TAGCACATACTATGCAGAGTCCGTGAAGGGCCGATTCACCATC |
| | | TCCAGAGATAACCCCAAGAACACGGTGTATTTACAAATGAACA |
| | | GCCTGAAACCTGAGGACACGGCCGTTTATTACTGTGCAGCAAC |
| | | TAGGACATCCTGGACCGAGAATACCTACTGGGGCCAGGGGACC |
| | | CAGGTCACCGTTTCCTCACACCACCACCACCACCACTAG |
| 79 | Exemplary Bispecific T cell | MEFGLSWVFLVALFRGVQCTGGSSQVQLVESGGGLVQAGGSLR |
| | engager construct | LSCAASGRTVSTYNMAWFRQAPGKEREIVAVINWTGGSTYYAE |
| | Amino Acid sequence | SVKGRFTISRDNPKNTVYLQMNSLKPEDTAVYYCAATRTSWTE |
| | bioTP7-7-CD3-1E2 | NTYWGQGTQVTVSSEDFLGGGGSQVQLQQPGAELGKPGTSVKL |
| | | SCKASGYTFTSYWIHWVKQRPGQGLEWVGNINPNSGSINYNEK |
| | | FKNKATLTVDKSSSTAYMQLSTLTSEDFAVYYCTRDTSGQYYF |
| | | DYWGQGTTLTVSSGGGGSGGGGSGGGGSDIVMSQSPSSLAVSA |
| | | GERVTMSCKSSQSLLNSRTRKNYLAWYQQKPGQSPKLLIYWAS |
| | | TRESGVPDRFTGSGSGTDFTLTISSVQAEDLAVYYCIQSYHLR |
| | | TFGGGTKLEIKHHHHHH** |
| 80 | Exemplary alternative Bi- | MEFGLSWVFLVALFRGVQCTGQVQLQQPGAELGKPGTSVKLSC |
| | specific | KASGYTFTSYWIHWVKQRPGQGLEWVGNINPNSGSINYNEKFK |
| | T cell engager format | NKATLTVDKSSSTAYMQLSTLTSEDFAVYYCTRDTSGQYYFDY |
| | CD3-1E2-bioTP7-7 | WGQGTTLTVSSGGGGSGGGGSGGGGSDIVMSQSPSSLAVSAGE |
| | | RVTMSCKSSQSLLNSRTRKNYLAWYQQKPGQSPKLLIYWASTR |
| | | ESGVPDRFTGSGSGTDFTLTISSVQAEDLAVYYCIQSYHLRTF |
| | | GGGTKLEIKGLGGGGSQVQLVESGGGLVQAGGSLRLSCAASGR |
| | | TVSTYNMAWFRQAPGKEREIVAVINWTGGSTYYAESVKGRFTI |
| | | SRDNPKNTVYLQMNSLKPEDTAVYYCAATRTSWTENTYWGQGT |
| | | QVTVSSHHHHHH** |
| 81 | Exemplary Human CD3- | QVQLQQPGAELGKPGTSVKLSCKASGYTFTSYWIHWVKQRPGQ |
| | targeting single chain | GLEWVGNINPNSGSINYNEKFKNKATLTVDKSSSTAYMQLSTL |
| | variable domain | TSEDFAVYYCTRDTSGQYYFDYWGQGTTLTVSSGGGGSGGGGS |
| | | GGGGSDIVMSQSPSSLAVSAGERVTMSCKSSQSLLNSRTRKNY |
| | | LAWYQQKPGQSPKLLIYWASTRESGVPDRFTGSGSGTDFTLTI |
| | | SSVQAEDLAVYYCIQSYHLRTFGGGTKLEIK |
| 82 | Bispecific T cell engager | ATGGAGTTTGGGCTGAGCTGGGTTTTCCTCGTTGCTCTTTTTA |
| | modular construct DNA | GAGGTGTCCAGTGTACAGGAGGGTCTTCG[sdAb_cds]gaag |
| | sequence | acttccttggaggaggcggaagtCAAGTGCAGTTGCAACAACC |
| | (“sdAb_cds” indicates the | AGGTGCCGAGCTTGGGAAACCGGGTACCAGCGTTAAACTCTCT |
| | coding | TGCAAAGCATCCGGTTATACCTTTACTTCTTACTGGATCCATT |
| | sequence for any one of the | GGGTCAAACAACGCCCAGGCCAGGGTCTCGAATGGGTGGGCAA |
| | sdAbs as described herein) | CATCAACCCAAACAGCGGCTCTATCAATTATAACGAAAAGTTT |
| | | AAGAATAAAGCAACTCTCACGGTGGATAAGAGCAGCTCAACCG |
| | | CTTATATGCAATTGAGTACTTTGACCAGTGAAGATTTCGCTGT |
| | | GTATTATTGCACGCGCGATACATCCGGACAGTATTATTTCGAT |
| | | TATTGGGGCCAGGGGACAACCCTGACAGTGTCTAGCggcggtg |
| | | gtgggtcaggcggcggtgggagcggaggaggtggaagcGATAT |
| | | CGTAATGAGTCAATCCCCTAGCAGCTTGGCGGTAAGTGCTGGG |
| | | GAACGCGTTACAATGTCCTGTAAGAGCTCTCAATCTTTGCTGA |
| | | ATTCCAGGACTAGGAAGAATTATCTGGCCTGGTATCAACAAAA |
| | | GCCCGGACAAAGTCCAAAGCTCCTGATTTATTGGGCTAGCACC |
| | | AGAGAGTCAGGAGTACCAGACCGGTTTACTGGAAGCGGGTCAG |
| | | GAACCGACTTTACCCTGACAATATCCAGCGTCCAAGCAGAAGA |
| | | TCTTGCCGTGTACTATTGTATTCAGTCCTACCACTTGCGCACT |
| | | TTTGGGGGGGGAACAAAACTCGAGATAAAGCACCACCACCACC |
| | | ACCACTAG |
| 83 | Exemplary Dimeric Bi- | MDWTWRFLFVVAAATGVQSTGQVQLVESGGGLVQAGGSLRLSC |
| | specific T | AASGRTVSTYNMAWFRQAPGKEREIVAVINWTGGSTYYAESVK |
| | cell engager format | GRFTISRDNPKNTVYLQMNSLKPEDTAVYYCAATRTSWTENTY |
| | bioTP7-7-CD3-1E2scFv-huFc | WGQGTQVTVSSPLGRPGGGGSGGGGSGGGGSGGTTTPAPRPPT |
| | | PAPTIASQPLSLRPEACRPAAGGAVHTRGLDFACDGSLPSGGG |
| | | GSDIVMSQSPSSLAVSAGERVTMSCKSSQSLLNSRTRKNYLAW |
| | | YQQKPGQSPKLLIYWASTRESGVPDRFTGSGSGTDFTLTISSV |
| | | QAEDLAVYYCIQSYHLRTFGGGTKLEIKGGGGSGGGGSGGGGS |
| | | QVQLQQPGAELGKPGTSVKLSCKASGYTFTSYWIHWVKQRPGQ |
| | | GLEWVGNINPNSGSINYNEKFKNKATLTVDKSSSTAYMQLSTL |
| | | TSEDFAVYYCTRDTSGQYYFDYWGQGTTLTVSSGGGGSEPKSS |
| | | DKTHTCPPCPAPELLGGPSVFLFPPKPKDTLMISRTPEVTCVV |
| | | VDVSHEDPEVKFNWYVDGVEVHNAKTKPREEQYNSTYRVVSVL |
| | | TVLHQDWLNGKEYKCKVSNKALPAPIEKTISKAKGQPREPQVY |
| | | TLPPSREEMTKNQVSLTCLVKGFYPSDIAVEWESNGQPENNYK |
| | | TTPPVLDSDGSFFLYSKLTVDKSRWQQGNVFSCSVMHEALHNH |
| | | YTQKSLSLSPG* |
| 84 | Exemplary human-Fc domain | EPKSSDKTHTCPPCPAPELLGGPSVFLFPPKPKDTLMISRTPE |
| | | VTCVVVDVSHEDPEVKFNWYVDGVEVHNAKTKPREEQYNSTYR |
| | | VVSVLTVLHQDWLNGKEYKCKVSNKALPAPIEKTISKAKGQPR |
| | | EPQVYTLPPSREEMTKNQVSLTCLVKGFYPSDIAVEWESNGQP |
| | | ENNYKTTPPVLDSDGSFFLYSKLTVDKSRWQQGNVFSCSVMHE |
| | | ALHNHYTQKSLSLSPG |
| 85 | Exemplary MSLN-targeting NK- | QVQLVESGGGLVQAGGSLRLSCAASGRTVSTYNMAWFRQAPGK |
| | engaging bispecific engager | EREIVAVINWTGGSTYYAESVKGRFTISRDNPKNTVYLQMNSL |
| | format | KPEDTAVYYCAATRTSWTENTYWGQGTQVTVSSGGGGSGGGGS |
| | bioTP7-7-(G4S)3-[NK-engaging | GGGGSAS[NK-engaging Ab. fragment sequence] |
| | antibody fragment] | |
| 86 | CDR1 Consensus (7-5, 7-56) | GRX1X2SX3X4F (X1 = S or D; X2 = I or F, |
| | | X3 = D or S, X4 = Y or H) |
| 87 | CDR2 Consensus (7-5, 7-56) | ISWSGGTX1 (X1 = A or T) |
| 88 | CDR3 Consensus (7-5, 7-56) | AARX1X2YDSRTSLX3NSYDYX4Y (X1 = L or R, X2 = S |
| | | or T, X3 = Q or R, X4 = N or P) |

1. An isolated single domain antibody (sdAb), or a construct thereof comprising said sdAb, which sdAb binds specifically to human mesothelin (hMSLN), the sdAb comprising
i) a CDR1 amino acid sequence as set forth in SEQ ID NO: 86, 25, 4, 22, 1, 7, 10, 13, 16, 19, 28, 31, 34, 37, 40 or 43;
ii) a CDR2 amino acid sequence as set forth in SEQ ID NO: 87, 26, 5, 23, 2, 8, 11, 14, 17, 20, 29, 32, 35, 38, 41, or 44; and
iii) a CDR3 amino acid sequence as set forth in SEQ ID NO: 88, 27, 6, 24, 3, 9, 12, 15, 18, 21, 30, 33, 36, 39, 42, or 45; or
CDR1, CDR2, and CDR3 amino acid sequences that are, as a group, at least 80%, at least 85%, or at least 90% identical to the respective CDR1, CDR2, and CDR3 amino acid sequences, as a group, selected from parts i)-iii).

2. The sdAb or the construct thereof of claim 1, the sdAb comprising:
i) a CDR1 amino acid sequence as set forth in SEQ ID NO:86, a CDR2 amino acid sequence as set forth in SEQ ID NO: 87, and a CDR3 amino acid sequence as set forth in SEQ ID NO: 88,
ii) a CDR1 amino acid sequence as set forth in SEQ ID NO:4, a CDR2 amino acid sequence as set forth in SEQ ID NO: 5, and a CDR3 amino acid sequence as set forth in SEQ ID NO: 6,
iii) a CDR1 amino acid sequence as set forth in SEQ ID NO: 25, a CDR2 amino acid sequence as set forth in SEQ ID NO: 26, and a CDR3 amino acid sequence as set forth in SEQ ID NO: 27,
iv) a CDR1 amino acid sequence as set forth in SEQ ID NO: 22, a CDR2 amino acid sequence as set forth in SEQ ID NO: 23, and a CDR3 amino acid sequence as set forth in SEQ ID NO: 24,
v) a CDR1 amino acid sequence as set forth in SEQ ID NO: 1, a CDR2 amino acid sequence as set forth in SEQ ID NO: 2, and a CDR3 amino acid sequence as set forth in SEQ ID NO: 3,
vi) a CDR1 amino acid sequence as set forth in SEQ ID NO: 7, a CDR2 amino acid sequence as set forth in SEQ ID NO: 8, and a CDR3 amino acid sequence as set forth in SEQ ID NO: 9,
vii) a CDR1 amino acid sequence as set forth in SEQ ID NO: 10, a CDR2 amino acid sequence as set forth in SEQ ID NO: 11, and a CDR3 amino acid sequence as set forth in SEQ ID NO: 12,
viii) a CDR1 amino acid sequence as set forth in SEQ ID NO: 13, a CDR2 amino acid sequence as set forth in SEQ ID NO: 14, and a CDR3 amino acid sequence as set forth in SEQ ID NO: 15,
ix) a CDR1 amino acid sequence as set forth in SEQ ID NO: 16, a CDR2 amino acid sequence as set forth in SEQ ID NO: 17, and a CDR3 amino acid sequence as set forth in SEQ ID NO: 18,
x) a CDR1 amino acid sequence as set forth in SEQ ID NO: 19, a CDR2 amino acid sequence as set forth in SEQ ID NO: 20, and a CDR3 amino acid sequence as set forth in SEQ ID NO: 21,
xi) a CDR1 amino acid sequence as set forth in SEQ ID NO: 28, a CDR2 amino acid sequence as set forth in SEQ ID NO: 29, and a CDR3 amino acid sequence as set forth in SEQ ID NO: 30,
xii) a CDR1 amino acid sequence as set forth in SEQ ID NO: 31, a CDR2 amino acid sequence as set forth in SEQ ID NO: 32, and a CDR3 amino acid sequence as set forth in SEQ ID NO: 33,),
xiii) a CDR1 amino acid sequence as set forth in SEQ ID NO: 34, a CDR2 amino acid sequence as set forth in SEQ ID NO: 35, and a CDR3 amino acid sequence as set forth in SEQ ID NO: 36,
xiv) a CDR1 amino acid sequence as set forth in SEQ ID NO: 37, a CDR2 amino acid sequence as set forth in SEQ ID NO: 38, and a CDR3 amino acid sequence as set forth in SEQ ID NO: 39,
xv) a CDR1 amino acid sequence as set forth in SEQ ID NO: 40, a CDR2 amino acid sequence as set forth in SEQ ID NO: 41, and a CDR3 amino acid sequence as set forth in SEQ ID NO: 42,
xvi) a CDR1 amino acid sequence as set forth in SEQ ID NO: 43, a CDR2 amino acid sequence as set forth in SEQ ID NO: 44, and a CDR3 amino acid sequence as set forth in SEQ ID NO: 45; or
xvii) CDR1, CDR2, and CDR3 amino acid sequences that are, as a group, at least 80%, at least 85%, or at least 90% identical to the respective CDR1, CDR2, and CDR3 amino acid sequences, as a group, selected from parts i)-xvi).

3. The sdAb or the construct thereof of claim 1, the sdAb comprising:
i) a CDR1 amino acid sequence as set forth in SEQ ID NO: 86; a CDR2 amino acid sequence as set forth in SEQ ID NO: 87, and a CDR3 amino acid sequence as set forth in SEQ ID NO: 88; or
ii) a CDR1 amino acid sequence as set forth in SEQ ID NO: 25; a CDR2 amino acid sequence as set forth in SEQ ID NO: 26, and a CDR3 amino acid sequence as set forth in SEQ ID NO: 27.

4. The sdAb or construct thereof of any one of claims 1 to 3, wherein the sdAb is humanized.

5. The sdAb or construct thereof of any one of claims 1 to 4, wherein the sdAb has a binding affinity (K_{D}) for hMSLN of 5.8×10⁻⁸ M or less, more preferably 3×10⁻⁸ M or less, more preferably 2×10⁻⁹ M or less, more preferably 6×10⁻¹⁰ M or less, more preferably 5.79×10⁻¹¹ M or less.

6. The sdAb or construct thereof of any one of claims 1 to 5, wherein the construct is a multivalent antibody comprising:
a first antigen-binding portion comprising the sdAb, and
a second antigen-binding portion.

7. The sdAb or construct thereof of claim 6, wherein the second antigen-binding portion is selected from the group consisting of an scFv, a second sdAb, an aptamer, a protein receptor, or a cytokine.

8. The sdAb or construct thereof of claim 6 or 7, wherein the second antigen-binding portion binds specifically to a cell-surface marker of an immune cell.

9. The sdAb or construct thereof of claim 8, wherein the cell surface marker is selected from the group consisting of a T-cell marker, NK-cell marker, or a T- and NK-cell marker.

10. The sdAb or construct thereof of claim 9, wherein the T-cell marker comprises human CD3, and wherein the NK-cell marker is human NKp30.

11. The sdAb or construct thereof of any one of claims 6 to 10, wherein the multivalent antibody is a dimeric immune-cell engager, said multivalent antibody further comprising a human Fc domain.

12. The sdAb or construct thereof of claim 6, wherein the multivalent antibody is encoded by SEQ ID NO: 82 or SEQ ID NO: 83.

13. The sdAb or construct thereof of any one of claims 6 to 12, further comprising a third antigen-binding portion that binds to an antigen target that may or may not be distinct from hMSLN.

14. The sdAb or construct thereof of claim 13, wherein the third antigen-binding portion binds to human serum albumin to extend serum half-life.

15. The sdAb or construct thereof of any one of claims 1 to 5, wherein the sdAb construct is a chimeric antibody receptor (CAR), which specifically binds to human mesothelin (hMSLN).

16. The sdAb or construct thereof of claim 15, wherein the CAR comprises, in an N-terminal to C-terminal direction:
the sdAb,
a polypeptide hinge,
a transmembrane domain, and
a cytoplasmic domain comprising at least one signaling domain, preferably wherein the cytoplasmic domain further comprises a co-stimulatory domain.

17. The sdAb or construct thereof of claim 15 or 16, wherein the CAR comprises the amino acid sequence of any one of SEQ ID NOs: 61 to 66 or an amino acid sequence that is at least 80%, at last 85%, at least 90%, or at least 95% identical thereto.

18. The sdAb or construct thereof of claim 16 or 17, wherein the CAR further comprises a second binding domain.

19. A nucleic acid molecule encoding the sdAb or construct thereof of any one of claims 1 to 18.

20. A recombinant viral particle comprising the nucleic acid molecule as defined in claim 19.

21. A cell comprising the nucleic acid molecule as defined in claim 19.

22. An engineered cell expressing at the cell surface membrane the CAR as defined in any one of claims 15 to 18.

23. The engineered cell of claim 22,
which is an immune cell, preferably a leukocyte, more preferably a T-cell, a monocyte, a macrophage, or a neutrophil;
which is an induced pluripotent stem cell (iPSC), or differentiated cell product derived thereof; or
which is an immune cell derived from T-lymphocytes.

24. A use, for the treatment of a cancer, of
the sdAb or construct thereof as defined in any one of claims 1 to 18;
the nucleic acid molecule as defined in claim 19;
the recombinant viral particle as defined in claim 20;
the cell as defined in claim 21; or
the engineered cell as defined in claim 22 or 23.

25. A method of treating a cancer in subject comprising administering to the subject:
the sdAb or construct thereof as defined in any one of claims 1 to 18;
the nucleic acid molecule as defined in claim 19;
the recombinant viral particle as defined in claim 20;
the cell as defined in claim 21; or
the engineered cell as defined in claim 22 or 23.

26. The use of claim 24 or the method of claim 25, where in the cancer comprises cells that aberrantly over-express MSLN compared to corresponding healthy cells.
